(19) United States
(12) Patent Application Publication
(10) Pub. No.: US 2025/0194571 A1
(43) Pub. Date: Jun. 19, 2025

(54) COMPOSITIONS AND METHODS FOR DEFINING OPTIMAL TREATMENT TIMEFRAMES IN LYSOSOMAL DISEASE

(71) Applicant: Regeneron Pharmaceuticals, Inc., Tarrytown, NY (US)

(72) Inventors: Elizabeth Hwang, Great Neck, NY (US); Katherine Cygnar, New York, NY (US); Jeremy Rabinowitz, Cortlandt Manor, NY (US); Jose F. Rojas, Newburgh, NY (US); Aristides Economides, Tarrytown, NY (US)

(73) Assignee: Regeneron Pharmaceuticals, Inc., Tarrytown, NY (US)

(21) Appl. No.: 18/836,094

(22) PCT Filed: Feb. 7, 2023
(86) PCT No.: PCT/US2023/062153

§ 371 Date: Aug. 6, 2024

Related U.S. Application Data
(60) Provisional application No. 63/307,431, filed on Feb. 7, 2022.

(51) Int. Cl. A01K 67/0276; C12N 9/12

(52) U.S. Cl. CPC A01K 67/0276; C12N 9/1241; A01K 2217/075; A01K 2217/203; A01K 2227/105; A01K 2267/0306

(57) ABSTRACT

Non-human animals comprising a reverse-conditional null endogenous lysosomal storage disease gene and methods of making such non-human animals are provided. Also provided are methods of using the non-human animals for assessing reversibility of a phenotype of the lysosomal storage disease or for determining an optimal timeframe for treatment of a phenotype of the lysosomal storage disease.

### CROSS-REFERENCE TO RELATED APPLICATIONS

This application claims the benefit of U.S. Application No. 63/307,431, filed Feb. 7, 2022, which is herein incorporated by reference in its entirety for all purposes.

### REFERENCE TO A SEQUENCE LISTING SUBMITTED AS AN XML FILE VIA EFS WEB

The Sequence Listing written in file 057766-590926SEQLIST.txt is 734 kilobytes, was created on Feb. 3, 2023, and is hereby incorporated by reference.

### BACKGROUND

Mucopolysaccharidosis VI (MPS VI), also known as Maroteaux-Lamy Syndrome, is a lysosomal disease resulting from impaired function of the arylsulfatase B (ARSB) protein. This impairment causes aberrant accumulation of dermatan sulfate, a glycosaminoglycan (GAG) abundant in growth plates, cartilage, and extracellular matrix. While clinical presentation is variable in terms of age at first symptom manifestation and disease severity, MPS VI classically presents at early ages and significantly impacts the skeleton. Current treatment guidelines recommend enzyme replacement therapy, which is known to provide incomplete or ineffective recovery from the skeletal manifestations of disease. We can postulate this may be due to the inability of the exogenous enzyme to reach affected cells, or that disease may not be reversible at the time therapy is delivered. To date, no models of disease exist that separate treatment efficacy from disease reversibility. Similar issues exist with other mucopolysaccharidosis diseases and other lysosomal storage diseases.

### SUMMARY

Non-human animals, non-human animal cells, and non-human animal genomes comprising a reverse-conditional null endogenous lysosomal storage disease gene are provided, as well as methods of making and using such non-human animals, non-human animal cells, and non-human animal genomes. Also provided are reverse-conditional null endogenous lysosomal storage disease genes, nucleic acids (e.g. isolated nucleic acids) comprising reverse-conditional null endogenous lysosomal storage disease genes, nuclease agents and/or targeting vectors for use in making reverse-conditional null endogenous lysosomal storage disease genes, and methods of making and using such reverse-conditional null endogenous lysosomal storage disease genes.

In one aspect, provided are non-human animals, non-human animal cells, and non-human animal genomes in their genome a reverse-conditional null endogenous lysosomal storage disease gene. In some such non-human animals, non-human animal cells, and non-human animal genomes, the reverse-conditional null endogenous lysosomal storage disease gene is a null endogenous lysosomal storage disease gene that is converted to a functional endogenous lysosomal storage disease gene upon treatment with a recombinase. In some such non-human animals, non-human animal cells, and non-human animal genomes, a critical region of the endogenous lysosomal storage disease gene is inverted and flanked by recombinase recognition sites in the reverse-conditional null endogenous lysosomal storage disease gene, wherein the recombinase recognition sites are oriented such that the critical region is reinverted upon treatment with the recombinase. In some such non-human animals, non-human animal cells, and non-human animal genomes, the critical region comprises a coding sequence of the endogenous lysosomal storage disease gene.

In some such non-human animals, non-human animal cells, and non-human animal genomes, the recombinase is a Cre recombinase. In some such non-human animals, non-human animal cells, and non-human animal genomes, the recombinase recognition sites comprise a lox71 site and a lox66 site.

Some such non-human animals, non-human animal cells, and non-human animal genomes further comprise a genomically integrated recombinase expression cassette encoding the recombinase. In some such non-human animals, non-human animal cells, and non-human animal genomes, the genomically integrated recombinase expression cassette comprises a recombinase coding sequence operably linked to a tissue-specific promoter. In some such non-human animals, non-human animal cells, and non-human animal genomes, the recombinase is inducible. In some such non-human animals, non-human animal cells, and non-human animal genomes, the recombinase is inducible upon treatment with tamoxifen.

In some such non-human animals, non-human animal cells, and non-human animal genomes, the reverse-conditional null endogenous lysosomal storage disease gene comprises a selection cassette or a reporter gene flanked by recombinase recognition sites for a second recombinase. In some such non-human animals, non-human animal cells, and non-human animal genomes, the reverse-conditional null endogenous lysosomal storage disease gene comprises the selection cassette, wherein the selection cassette is a self-deleting selection cassette. Some such non-human animals, non-human animal cells, and non-human animal genomes do not comprise a selection cassette or a reporter gene.

In some such non-human animals, non-human animal cells, and non-human animal genomes, the non-human animal comprises the reverse-conditional null endogenous lysosomal storage disease gene in its germline. In some such non-human animals, non-human animal cells, and non-human animal genomes, the non-human animal is a mammal. In some such non-human animals, non-human animal cells, and non-human animal genomes, the non-human animal is a rodent. In some such non-human animals, non-human animal cells, and non-human animal genomes, the non-human animal is a mouse or a rat. In some such non-human animals, non-human animal cells, and non-human animal genomes, the non-human animal is the mouse.

In some such non-human animals, non-human animal cells, and non-human animal genomes, the lysosomal storage disease gene is a mucopolysaccharidosis gene.

In some such non-human animals, non-human animal cells, and non-human animal genomes, the lysosomal storage disease gene is an Arsb gene. In some such non-human animals, non-human animal cells, and non-human animal genomes, a critical region of the endogenous Arsb gene is inverted and flanked by recombinase recognition sites in the reverse-conditional null endogenous Arsb gene, wherein the recombinase recognition sites are oriented such that the critical region is reinverted upon treatment with the recombinase. In some such non-human animals, non-human animal cells, and non-human animal genomes, the critical region comprises exon 5 of the endogenous Arsb gene. In some such non-human animals, non-human animal cells, and non-human animal genomes, the non-human animal displays one or more phenotypes associated with mucopolysaccharidosis VI. In some such non-human animals, non-human animal cells, and non-human animal genomes, the non-human animal displays one or more skeletal phenotypes associated with mucopolysaccharidosis VI. In some such non-human animals, non-human animal cells, and non-human animal genomes, the non-human animal displays one or more or all of the following relative to a wild type non-human animal: (a) increased accumulation of glycosaminoglycans in the liver; (b) increased accumulation of glycosaminoglycans in the heart; (c) increased accumulation of glycosaminoglycans in the kidney; (d) decreased tibia length; (e) decreased spinal column length; (f) increased tibial growth plate width; and (g) decreased cranial length:width ratio. In some such non-human animals, non-human animal cells, and non-human animal genomes, the non-human animal displays all of the following relative to the wild type non-human animal: (a) increased accumulation of glycosaminoglycans in the liver; (b) increased accumulation of glycosaminoglycans in the heart; (c) increased accumulation of glycosaminoglycans in the kidney; (d) decreased tibia length; (e) decreased spinal column length; (f) increased tibial growth plate width; and (g) decreased cranial length:width ratio.

In some such non-human animals, non-human animal cells, and non-human animal genomes, a region comprising exon 5 of the endogenous Arsb gene is inverted and flanked by recombinase recognition sites in the reverse-conditional null endogenous Arsb gene, the recombinase recognition sites are oriented such that the region is reinverted upon treatment with the recombinase, the recombinase is a Cre recombinase, and the recombinase recognition sites comprise a lox71 site and a lox66 site, the non-human animal comprises the reverse-conditional null endogenous Arsb gene in its germline, the non-human animal is a mouse, and the non-human animal displays one or more phenotypes associated with mucopolysaccharidosis VI. Optionally, the one or more phenotypes comprise one or more or all of the following relative to a wild type non-human animal: (a) increased accumulation of glycosaminoglycans in the liver; (b) increased accumulation of glycosaminoglycans in the heart; (c) increased accumulation of glycosaminoglycans in the kidney; (d) decreased tibia length; (e) decreased spinal column length; (f) increased tibial growth plate width; and (g) decreased cranial length:width ratio. In some such non-human animals, non-human animal cells, and non-human animal genomes, the non-human animal displays the decreased tibia length, wherein the decreased tibia length is rescuable within one month or three months after treatment with the recombinase at post-natal day 7 (or earlier) but is not rescuable within one month or three months after treatment with the recombinase at post-natal day 21 (or later) or at 8-10 weeks (e.g., 8 weeks) after birth. In some such non-human animals, non-human animal cells, and non-human animal genomes, the non-human animal displays the decreased spinal column length, wherein the decreased spinal column length is rescuable within one month or three months after treatment with the recombinase at post-natal day 7 (or earlier) but is not rescuable within one month or three months after treatment with the recombinase at post-natal day 21 (or later) or at 8-10 weeks (e.g., 8 weeks) after birth. In some such non-human animals, non-human animal cells, and non-human animal genomes, the non-human animal displays the increased tibial growth plate width, wherein the increased tibial growth plate width is rescuable within one month or three months after treatment with the recombinase at post-natal day 7 (or earlier) but is not rescuable within one month or three months after treatment with the recombinase at post-natal day 21 (or later) or at 8-10 weeks (e.g., 8 weeks) after birth. In some such non-human animals, non-human animal cells, and non-human animal genomes, the non-human animal displays the decreased cranial length:width ratio, wherein the decreased cranial length:width ratio is rescuable within one month or three months after treatment with the recombinase at post-natal day 7 (or earlier) but is not rescuable within one month or three months after treatment with the recombinase at post-natal day 21 (or later) or at 8-10 weeks (e.g., 8 weeks) after birth. In some such non-human animals, non-human animal cells, and non-human animal genomes, the non-human animal displays the decreased tibia length, wherein the decreased tibia length is rescued within one month or three months after treatment with the recombinase at post-natal day 7 (or earlier). In some such non-human animals, non-human animal cells, and non-human animal genomes, the non-human animal displays the decreased tibia length, wherein the decreased tibia length is not rescued within one month or three months after treatment with the recombinase at post-natal day 21 (or later) or at 8-10 weeks (e.g., 8 weeks) after birth. In some such non-human animals, non-human animal cells, and non-human animal genomes, the non-human animal displays the decreased spinal column length, wherein the decreased spinal column length is rescued within one month or three months after treatment with the recombinase at post-natal day 7 (or earlier). In some such non-human animals, non-human animal cells, and non-human animal genomes, the non-human animal displays the decreased spinal column length, wherein the decreased spinal column length is not rescued within one month or three months after treatment with the recombinase at post-natal day 21 (or later) or at 8-10 weeks (e.g., 8 weeks) afterbirth. In some such non-human animals, non-human animal cells, and non-human animal genomes, the non-human animal displays the increased tibial growth plate width, wherein the increased tibial growth plate width is rescued within one month or three months after treatment with the recombinase at post-natal day 7 (or earlier). In some such non-human animals, non-human animal cells, and non-human animal genomes, the non-human animal displays the increased tibial growth plate width, the increased tibial growth plate width is not rescued within one month or three months after treatment with the recombinase at post-natal day 21 (or later) or at 8-10 weeks (e.g., 8 weeks) after birth. In some such non-human animals, non-human animal cells, and non-human animal genomes, the non-human animal displays the decreased cranial length:width ratio, wherein the decreased cranial length:width ratio is rescued within one month or three months after treatment with the recombinase at post-natal day 7 (or earlier). In some such non-human animals, non-human animal cells, and non-human animal genomes, the non-human animal displays the decreased cranial length:width ratio, wherein the decreased cranial length:width ratio is not rescued within one month or three months after treatment with the recombinase at post-natal day 21 (or later) or at 8-10 weeks (e.g., 8 weeks) after birth. In some such non-human animals, non-human animal cells, and non-human animal genomes, the reverse-conditional null endogenous Arsb gene comprises the sequence set forth in SEQ ID NO: 7 or 8. In some such non-human animals, non-human animal cells, and non-human animal genomes, the reverse-conditional null endogenous Arsb gene comprises the sequence set forth in SEQ ID NO: 47. In some such non-human animals, non-human animal cells, and non-human animal genomes, upon treatment the reverse-conditional null endogenous Arsb gene comprises the sequence set forth in SEQ ID NO: 9. In some such non-human animals, non-human animal cells, and non-human animal genomes, upon treatment the reverse-conditional null endogenous Arsb gene comprises the sequence set forth in SEQ ID NO: 48.

In some such non-human animals, non-human animal cells, and non-human animal genomes, the lysosomal storage disease gene is an Idua gene. In some such non-human animals, non-human animal cells, and non-human animal genomes, a critical region of the endogenous Idua gene is inverted and flanked by recombinase recognition sites in the reverse-conditional null endogenous Idua gene, wherein the recombinase recognition sites are oriented such that the critical region is reinverted upon treatment with the recombinase. In some such non-human animals, non-human animal cells, and non-human animal genomes, the critical region comprises a region from exon 2 to the stop codon of the endogenous Idua gene. In some such non-human animals, non-human animal cells, and non-human animal genomes, the non-human animal displays one or more phenotypes associated with mucopolysaccharidosis I. In some such non-human animals, non-human animal cells, and non-human animal genomes, the non-human animal displays one or more skeletal phenotypes associated with mucopolysaccharidosis I. In some such non-human animals, non-human animal cells, and non-human animal genomes, the non-human animal displays one or more neurological phenotypes associated with mucopolysaccharidosis I. In some such non-human animals, non-human animal cells, and non-human animal genomes, the non-human animal displays one or more skeletal phenotypes associated with mucopolysaccharidosis I and one or more neurological phenotypes associated with mucopolysaccharidosis I. In some such non-human animals, non-human animal cells, and non-human animal genomes, the non-human animal displays one or more or all of the following relative to a wild type non-human animal: (a) increased accumulation of glycosaminoglycans in the liver; (b) increased accumulation of glycosaminoglycans in the heart; (c) increased accumulation of glycosaminoglycans in the kidney; (d) decreased tibia length; (e) decreased spinal column length; (f) decreased ribcage width; and (g) decreased cranial length:width ratio. In some such non-human animals, non-human animal cells, and non-human animal genomes, the non-human animal displays all of the following relative to the wild type non-human animal: (a) increased accumulation of glycosaminoglycans in the liver; (b) increased accumulation of glycosaminoglycans in the heart; (c) increased accumulation of glycosaminoglycans in the kidney; (d) decreased tibia length; (e) decreased spinal column length; (f) decreased ribcage width; and (g) decreased cranial length:width ratio. In some such non-human animals, non-human animal cells, and non-human animal genomes, the non-human animal displays one or more or all of the following relative to a wild type non-human animal: (a) decreased exploratory behavior; (b) decreased anxiety-related behavior; (c) impaired long-term memory retention; and (d) increased escape latency. In some such non-human animals, non-human animal cells, and non-human animal genomes, the non-human animal displays all of the following relative to the wild type non-human animal: (a) decreased exploratory behavior; (b) decreased anxiety-related behavior; (c) impaired long-term memory retention; and (d) increased escape latency.

In some such non-human animals, non-human animal cells, and non-human animal genomes, a region comprising exon 2 to the stop codon of the endogenous Idua gene is inverted and flanked by recombinase recognition sites in the reverse-conditional null endogenous Idua gene, the recombinase recognition sites are oriented such that the region is reinverted upon treatment with the recombinase, the recombinase is a Cre recombinase, and the recombinase recognition sites comprise a lox71 site and a lox66 site, the non-human animal comprises the reverse-conditional null endogenous Idua gene in its germline, the non-human animal is a mouse, and the non-human animal displays one or more phenotypes associated with mucopolysaccharidosis I. In some such non-human animals, non-human animal cells, and non-human animal genomes, the non-human animal displays one or more skeletal phenotypes associated with mucopolysaccharidosis I. In some such non-human animals, non-human animal cells, and non-human animal genomes, the non-human animal displays one or more neurological phenotypes associated with mucopolysaccharidosis I. In some such non-human animals, non-human animal cells, and non-human animal genomes, the non-human animal displays one or more skeletal phenotypes associated with mucopolysaccharidosis I and one or more neurological phenotypes associated with mucopolysaccharidosis I. In some such non-human animals, non-human animal cells, and non-human animal genomes, the non-human animal displays one or more or all of the following relative to a wild type non-human animal: (a) increased accumulation of glycosaminoglycans in the liver; (b) increased accumulation of glycosaminoglycans in the heart; (c) increased accumulation of glycosaminoglycans in the kidney; (d) decreased tibia length; (e) decreased spinal column length; (f) decreased ribcage width; and (g) decreased cranial length:width ratio. In some such non-human animals, non-human animal cells, and non-human animal genomes, the non-human animal displays one or more or all of the following relative to a wild type non-human animal: (a) decreased exploratory behavior; (b) decreased anxiety-related behavior; (c) impaired long-term memory retention; and (d) increased escape latency. In some such non-human animals, non-human animal cells, and non-human animal genomes, the reverse-conditional null endogenous Idua gene comprises the sequence set forth in SEQ ID NO: 10 or 11. In some such non-human animals, non-human animal cells, and non-human animal genomes, the reverse-conditional null endogenous Idua gene comprises the sequence set forth in SEQ ID NO: 49. In some such non-human animals, non-human animal cells, and non-human animal genomes, upon treatment the reverse-conditional null endogenous Idua gene comprises the sequence set forth in SEQ ID NO: 12. In some such non-human animals, non-human animal cells, and non-human animal genomes, upon treatment the reverse-conditional null endogenous Idua gene comprises the sequence set forth in SEQ ID NO: 50.

In another aspect, provided are methods of assessing reversibility of a phenotype of a lysosomal storage disease. Some such methods comprise: (a) treating any of the above non-human animals comprising in their genome a reverse-conditional null endogenous lysosomal storage disease gene with the recombinase at a selected time point after birth or treating the non-human animal with a candidate therapeutic agent at the selected time point after birth; and (b) assessing the phenotype of the lysosomal storage disease after a defined time period post-treatment relative to a control non-human animal.

In some such methods, step (a) comprises treating the non-human animal with the candidate therapeutic agent at the selected time point after birth. Optionally, the candidate therapeutic agent is a protein encoded by the endogenous lysosomal storage disease gene or a nucleic acid encoding the protein. In some such methods, the control non-human animal is the non-human animal in step (a) prior to treatment with the candidate therapeutic agent. In some such methods, the control non-human animal is a second non-human animal comprising the reverse-conditional null endogenous lysosomal storage disease gene, wherein the second non-human animal has not been treated with the candidate therapeutic agent. Optionally, the second non-human animal is the same age as the non-human animal in step (a).

In some such methods, step (a) comprises treating the non-human animal with the recombinase at the selected time point after birth. In some such methods, the non-human animal further comprises a genomically integrated recombinase expression cassette encoding the recombinase, wherein the recombinase is inducible, and step (a) comprises inducing the recombinase. In some such methods, the recombinase is inducible upon treatment with tamoxifen, and step (a) comprises treating the non-human animal with tamoxifen.

In some such methods, the control non-human animal is the non-human animal in step (a) prior to treatment with the recombinase. In some such methods, the control non-human animal is a second non-human animal comprising the reverse-conditional null endogenous lysosomal storage disease gene, wherein the second non-human animal has not been treated with the recombinase. In some such methods, the second non-human animal is the same age as the non-human animal in step (a). In some such methods, step (b) further comprises assessing the phenotype of the lysosomal storage disease after a defined time period post-treatment relative to a wild type non-human animal. Optionally, the wild type non-human animal is the same age as the non-human animal in step (b).

In some such methods, the defined time period post-treatment in step (b) is at least 1 week, at least 1 month, or at least 3 months after treatment. In some such methods, the defined time period post-treatment in step (b) is between about 1 month and about 3 months after treatment.

In some such methods, the lysosomal storage disease gene is a mucopolysaccharidosis gene, and the phenotype is a mucopolysaccharidosis phenotype. In some such methods, the lysosomal storage disease gene is Arsb, and the phenotype is a mucopolysaccharidosis VI phenotype. In some such methods, the phenotype is a skeletal mucopolysaccharidosis VI phenotype. In some such methods, the phenotype comprises, relative to a wild type non-human animal: (i) increased accumulation of glycosaminoglycans in the liver; (ii) increased accumulation of glycosaminoglycans in the heart; (iii) increased accumulation of glycosaminoglycans in the kidney; (iv) decreased tibia length; (v) decreased spinal column length; (vi) increased tibial growth plate width; or (vii) decreased cranial length:width ratio. In some such methods, the phenotype comprises the decreased tibia length, the decreased spinal column length, the increased tibial growth plate width, or the decreased cranial length:width ratio. In some such methods, the phenotype comprises the decreased tibia length or the decreased spinal column length. In some such methods, the lysosomal storage disease gene is Idua, and the phenotype is a mucopolysaccharidosis I phenotype. In some such methods, the phenotype is a skeletal mucopolysaccharidosis I phenotype. In some such methods, the phenotype comprises, relative to a wild type non-human animal: (i) increased accumulation of glycosaminoglycans in the liver; (ii) increased accumulation of glycosaminoglycans in the heart; (iii) increased accumulation of glycosaminoglycans in the kidney; (iv) decreased tibia length; (v) decreased spinal column length; (vi) decreased ribcage width; or (vii) decreased cranial length:width ratio. In some such methods, the phenotype is a neurological mucopolysaccharidosis I phenotype. In some such methods, the phenotype comprises, relative to a wild type non-human animal: (a) decreased exploratory behavior; (b) decreased anxiety-related behavior; (c) impaired long-term memory retention; or (d) increased escape latency.

In another aspect, provided are methods of determining an optimal timeframe for treatment of a phenotype of a lysosomal storage disease. Some such methods comprise: (a) performing any of the above methods of assessing reversibility of a phenotype of a lysosomal storage disease a first time in a first non-human animal, wherein the first non-human animal is treated with the recombinase at a first time point after birth; (b) performing the method of step (a) a second time in a second non-human animal, wherein the second non-human animal is treated with the recombinase at a second time point after birth, wherein the second time point is different from the first time point; and (c) comparing the phenotype in step (a) with the phenotype in step (b) and selecting the time point resulting in the better amelioration of the phenotype.

In some such methods, the lysosomal storage disease gene is a mucopolysaccharidosis gene, and the phenotype type is a mucopolysaccharidosis phenotype. In some such methods, the lysosomal storage disease gene is Arsb, and the phenotype is a mucopolysaccharidosis VI phenotype. In some such methods, the phenotype is a skeletal mucopolysaccharidosis VI phenotype. In some such methods, the phenotype comprises, relative to a wild type non-human animal: (i) increased accumulation of glycosaminoglycans in the liver; (ii) increased accumulation of glycosaminoglycans in the heart; (iii) increased accumulation of glycosaminoglycans in the kidney; (iv) decreased tibia length; (v) decreased spinal column length; (vi) increased tibial growth plate width; or (vii) decreased cranial length:width ratio. In some such methods, the phenotype comprises the decreased tibia length, the decreased spinal column length, the increased tibial growth plate width, or the decreased cranial length:width ratio. In some such methods, the phenotype comprises the decreased tibia length or the decreased spinal column length. In some such methods, the lysosomal storage disease gene is Idua, and the phenotype is a mucopolysaccharidosis I phenotype. In some such methods, the phenotype is a skeletal mucopolysaccharidosis I phenotype. In some such methods, the phenotype comprises, relative to a wild type non-human animal: (i) increased accumulation of glycosaminoglycans in the liver; (ii) increased accumulation of glycosaminoglycans in the heart; (iii) increased accumulation of glycosaminoglycans in the kidney; (iv) decreased tibia length; (v) decreased spinal column length; (vi) decreased ribcage width; or (vii) decreased cranial length:width ratio. In some such methods, the phenotype is a neurological mucopolysaccharidosis I phenotype. In some such methods, the phenotype comprises, relative to a wild type non-human animal: (a) decreased exploratory behavior; (b) decreased anxiety-related behavior; (c) impaired long-term memory retention; or (d) increased escape latency.

In another aspect, provided are methods of assessing the reversibility of a phenotype of a lysosomal storage disease by a combination therapy. Some such methods comprise: (a) performing any of the above methods of assessing reversibility of a phenotype of a lysosomal storage disease a first time in a first non-human animal and a second time in a second non-human animal, wherein the first non-human animal is treated with the recombinase but not the candidate therapeutic agent, and wherein the second non-human animal is treated with the recombinase and the candidate therapeutic agent; and (b) comparing the phenotype in the first non-human animal with the phenotype in the second non-human animal to determine if the combination of the recombinase and the candidate therapeutic agent results in better amelioration of the phenotype.

In some such methods, the second non-human animal is treated with the recombinase and the candidate therapeutic agent simultaneously. In some such methods, the second non-human animal is treated with the recombinase and the candidate therapeutic agent sequentially. In some such methods, the candidate therapeutic agent is not a protein encoded by the endogenous lysosomal storage disease gene or a nucleic acid encoding the protein.

In another aspect, provided are methods of assessing the efficacy of a candidate therapeutic agent for reversing a phenotype of a lysosomal storage disease. Some such methods comprise: (a) performing any of the above methods of assessing reversibility of a phenotype of a lysosomal storage disease a first time in a first non-human animal and a second time in a second non-human animal, wherein the first non-human animal is treated with the recombinase but not the candidate therapeutic agent, and wherein the second non-human animal is treated with the candidate therapeutic agent but not the recombinase; and (b) comparing the phenotype in the first non-human animal with the phenotype in the second non-human animal to determine if the recombinase results in better amelioration of the phenotype than the candidate therapeutic agent.

In some such methods, the recombinase and the candidate therapeutic agent are administered at the same selected time point after birth, and the phenotype is assessed after the same defined time period post-treatment in the first non-human animal and the second non-human animal. In some such methods, the candidate therapeutic agent is a protein encoded by the endogenous lysosomal storage disease gene or a nucleic acid encoding the protein.

In another aspect, provided are methods of making any of the above non-human animals comprising in their genome the reverse-conditional null endogenous lysosomal storage disease gene. Some such methods comprise: (a) introducing into a non-human animal host embryo a genetically modified non-human animal embryonic stem (ES) cell comprising in its genome the reverse-conditional null endogenous lysosomal storage disease gene; and (b) gestating the non-human animal host embryo in a surrogate non-human animal mother, wherein the surrogate non-human animal mother produces an F0 progeny genetically modified non-human animal comprising the reverse-conditional null endogenous lysosomal storage disease gene. Optionally, such methods can further comprise modifying the non-human animal ES cell prior to step (a) to comprise in its genome the reverse-conditional null endogenous lysosomal storage disease gene. Some such methods comprise gestating a genetically modified non-human animal host embryo in a non-human animal surrogate mother, wherein the genetically modified non-human animal host embryo comprises in its genome the reverse-conditional null endogenous lysosomal storage disease gene, and wherein the surrogate non-human animal mother produces an F0 progeny genetically modified non-human animal comprising the reverse-conditional null endogenous lysosomal storage disease gene. Optionally, such methods can further comprise modifying a non-human animal one-cell stage embryo to generate the genetically modified non-human animal host embryo prior to the gestating step.

Non-human animals, non-human animal cells, and non-human animal genomes comprising a reverse-conditional null endogenous Arsb gene are provided, as well as methods of making and using such non-human animals, non-human animal cells, and non-human animal genomes. Also provided are reverse-conditional null endogenous Arsb genes, nucleic acids (e.g. isolated nucleic acids) comprising reverse-conditional null endogenous Arsb genes, nuclease agents and/or targeting vectors for use in making reverse-conditional null endogenous Arsb genes, and methods of making and using such reverse-conditional null endogenous Arsb genes.

In one aspect, provided are non-human animals, non-human animal cells, and non-human animal genomes in their genome a reverse-conditional null endogenous Arsb gene. In some such non-human animals, non-human animal cells, and non-human animal genomes, the reverse-conditional null endogenous Arsb gene is a null endogenous Arsb gene that is converted to a functional endogenous Arsb gene upon treatment with a recombinase. In some such non-human animals, non-human animal cells, and non-human animal genomes, a critical region of the endogenous Arsb gene is inverted and flanked by recombinase recognition sites in the reverse-conditional null endogenous Arsb gene, wherein the recombinase recognition sites are oriented such that the critical region is reinverted upon treatment with the recombinase. In some such non-human animals, non-human animal cells, and non-human animal genomes, the critical region comprises a coding sequence of the endogenous Arsb gene. In some such non-human animals, non-human animal cells, and non-human animal genomes, the critical region comprises exon 5 of the endogenous Arsb gene. In some such non-human animals, non-human animal cells, and non-human animal genomes, the recombinase is a Cre recombinase. In some such non-human animals, non-human animal cells, and non-human animal genomes, the recombinase recognition sites comprise a lox71 site and a lox66 site.

Some such non-human animals, non-human animal cells, and non-human animal genomes further comprise a genomically integrated recombinase expression cassette encoding the recombinase. In some such non-human animals, non-human animal cells, and non-human animal genomes, the genomically integrated recombinase expression cassette comprises a recombinase coding sequence operably linked to a tissue-specific promoter. In some such non-human animals, non-human animal cells, and non-human animal genomes, the recombinase is inducible. In some such non-human animals, non-human animal cells, and non-human animal genomes, the recombinase is inducible upon treatment with tamoxifen.

In some such non-human animals, non-human animal cells, and non-human animal genomes, the reverse-conditional null endogenous Arsb gene comprises a selection cassette or a reporter gene flanked by recombinase recognition sites for a second recombinase. In some such non-human animals, non-human animal cells, and non-human animal genomes, the reverse-conditional null endogenous Arsb gene comprises the selection cassette, wherein the selection cassette is a self-deleting selection cassette. In some such non-human animals, non-human animal cells, and non-human animal genomes, the non-human animal does not comprise a selection cassette or a reporter gene.

In some such non-human animals, non-human animal cells, and non-human animal genomes, the non-human animal comprises the reverse-conditional null endogenous Arsb gene in its germline. In some such non-human animals, non-human animal cells, and non-human animal genomes, the non-human animal is a mammal. In some such non-human animals, non-human animal cells, and non-human animal genomes, the non-human animal is a rodent. In some such non-human animals, non-human animal cells, and non-human animal genomes, the non-human animal is a mouse or a rat. In some such non-human animals, non-human animal cells, and non-human animal genomes, the non-human animal is the mouse.

In some such non-human animals, non-human animal cells, and non-human animal genomes, the non-human animal displays one or more phenotypes associated with mucopolysaccharidosis VI. In some such non-human animals, non-human animal cells, and non-human animal genomes, the non-human animal displays one or more skeletal phenotypes associated with mucopolysaccharidosis VI. In some such non-human animals, non-human animal cells, and non-human animal genomes, the non-human animal displays one or more or all of the following relative to a wild type non-human animal: (a) increased accumulation of glycosaminoglycans in the liver; (b) increased accumulation of glycosaminoglycans in the heart; (c) increased accumulation of glycosaminoglycans in the kidney; (d) decreased tibia length; (e) decreased spinal column length; (f) increased tibial growth plate width; and (g) decreased cranial length:width ratio. In some such non-human animals, non-human animal cells, and non-human animal genomes, the non-human animal displays all of the following relative to the wild type non-human animal: (a) increased accumulation of glycosaminoglycans in the liver; (b) increased accumulation of glycosaminoglycans in the heart; (c) increased accumulation of glycosaminoglycans in the kidney; (d) decreased tibia length; (e) decreased spinal column length; (f) increased tibial growth plate width; and (g) decreased cranial length:width ratio.

In some such non-human animals, non-human animal cells, and non-human animal genomes, a region comprising exon 5 of the endogenous Arsb gene is inverted and flanked by recombinase recognition sites in the reverse-conditional null endogenous Arsb gene, the recombinase recognition sites are oriented such that the region is reinverted upon treatment with the recombinase, the recombinase is a Cre recombinase, and the recombinase recognition sites comprise a lox71 site and a lox66 site, the non-human animal comprises the reverse-conditional null endogenous Arsb gene in its germline, the non-human animal is a mouse, and the non-human animal displays one or more phenotypes associated with mucopolysaccharidosis VI. In some such non-human animals, non-human animal cells, and non-human animal genomes, the one or more phenotypes comprise one or more or all of the following relative to a wild type non-human animal: (a) increased accumulation of glycosaminoglycans in the liver; (b) increased accumulation of glycosaminoglycans in the heart; (c) increased accumulation of glycosaminoglycans in the kidney; (d) decreased tibia length; (e) decreased spinal column length; (f) increased tibial growth plate width; and (g) decreased cranial length:width ratio. In some such non-human animals, non-human animal cells, and non-human animal genomes, the non-human animal displays the decreased tibia length, wherein the decreased tibia length is rescuable within one month or three months after treatment with the recombinase at post-natal day 7 (or earlier) but is not rescuable within one month or three months after treatment with the recombinase at post-natal day 21 (or later) or at 8-10 weeks (e.g., 8 weeks) after birth. In some such non-human animals, non-human animal cells, and non-human animal genomes, the non-human animal displays the decreased spinal column length, wherein the decreased spinal column length is rescuable within one month or three months after treatment with the recombinase at post-natal day 7 (or earlier) but is not rescuable within one month or three months after treatment with the recombinase at post-natal day 21 (or later) or at 8-10 weeks (e.g., 8 weeks) after birth. In some such non-human animals, non-human animal cells, and non-human animal genomes, the non-human animal displays the increased tibial growth plate width, wherein the increased tibial growth plate width is rescuable within one month or three months after treatment with the recombinase at post-natal day 7 (or earlier) but is not rescuable within one month or three months after treatment with the recombinase at post-natal day 21 (or later) or at 8-10 weeks (e.g., 8 weeks) after birth. In some such non-human animals, non-human animal cells, and non-human animal genomes, the non-human animal displays the decreased cranial length:width ratio, wherein the decreased cranial length:width ratio is rescuable within one month or three months after treatment with the recombinase at post-natal day 7 (or earlier) but is not rescuable within one month or three months after treatment with the recombinase at post-natal day 21 (or later) or at 8-10 weeks (e.g., 8 weeks) after birth. In some such non-human animals, non-human animal cells, and non-human animal genomes, the non-human animal displays the decreased tibia length, wherein the decreased tibia length is rescued within one month or three months after treatment with the recombinase at post-natal day 7 (or earlier). In some such non-human animals, non-human animal cells, and non-human animal genomes, the non-human animal displays the decreased tibia length, wherein the decreased tibia length is not rescued within one month or three months after treatment with the recombinase at post-natal day 21 (or later) or at 8-10 weeks (e.g., 8 weeks) after birth. In some such non-human animals, non-human animal cells, and non-human animal genomes, the non-human animal displays the decreased spinal column length, wherein the decreased spinal column length is rescued within one month or three months after treatment with the recombinase at post-natal day 7 (or earlier). In some such non-human animals, non-human animal cells, and non-human animal genomes, the non-human animal displays the decreased spinal column length, wherein the decreased spinal column length is not rescued within one month or three months after treatment with the recombinase at post-natal day 21 (or later) or at 8-10 weeks (e.g., 8 weeks) afterbirth. In some such non-human animals, non-human animal cells, and non-human animal genomes, the non-human animal displays the increased tibial growth plate width, wherein the increased tibial growth plate width is rescued within one month or three months after treatment with the recombinase at post-natal day 7 (or earlier). In some such non-human animals, non-human animal cells, and non-human animal genomes, the non-human animal displays the increased tibial growth plate width, wherein the increased tibial growth plate width is not rescued within one month or three months after treatment with the recombinase at post-natal day 21 (or later) or at 8-10 weeks (e.g., 8 weeks) after birth. In some such non-human animals, non-human animal cells, and non-human animal genomes, the non-human animal displays the decreased cranial length:width ratio, wherein the decreased cranial length:width ratio is rescued within one month or three months after treatment with the recombinase at post-natal day 7 (or earlier). In some such non-human animals, non-human animal cells, and non-human animal genomes, the non-human animal displays the decreased cranial length:width ratio, wherein the decreased cranial length:width ratio is not rescued within one month or three months after treatment with the recombinase at post-natal day 21 (or later) or at 8-10 weeks (e.g., 8 weeks) after birth. In some such non-human animals, non-human animal cells, and non-human animal genomes, the reverse-conditional null endogenous Arsb gene comprises the sequence set forth in SEQ ID NO: 7 or 8. In some such non-human animals, non-human animal cells, and non-human animal genomes, the reverse-conditional null endogenous Arsb gene comprises the sequence set forth in SEQ ID NO: 47. In some such non-human animals, non-human animal cells, and non-human animal genomes, upon treatment the reverse-conditional null endogenous Arsb gene comprises the sequence set forth in SEQ ID NO: 9. In some such non-human animals, non-human animal cells, and non-human animal genomes, upon treatment the reverse-conditional null endogenous Arsb gene comprises the sequence set forth in SEQ ID NO: 48.

In another aspect, provided are methods of assessing reversibility of a phenotype of mucopolysaccharidosis VI. Some such methods comprise: (a) treating any of the above non-human animals comprising in their genome a reverse-conditional null endogenous Arsb gene with the recombinase at a selected time point after birth or treating the non-human animal with a candidate therapeutic agent at the selected time point after birth; and (b) assessing the phenotype of mucopolysaccharidosis VI after a defined time period post-treatment relative to a control non-human animal.

In some such methods, step (a) comprises treating the non-human animal with the candidate therapeutic agent at the selected time point after birth. Optionally, the candidate therapeutic agent is a protein encoded by the endogenous Arsb gene or a nucleic acid encoding the protein. In some such methods, the control non-human animal is the non-human animal in step (a) prior to treatment with the candidate therapeutic agent. In some such methods, the control non-human animal is a second non-human animal comprising the reverse-conditional null endogenous Arsb gene, wherein the second non-human animal has not been treated with the candidate therapeutic agent. Optionally, the second non-human animal is the same age as the non-human animal in step (a).

In some such methods, step (a) comprises treating the non-human animal with the recombinase at the selected time point after birth. In some such methods, the non-human animal further comprises a genomically integrated recombinase expression cassette encoding the recombinase, wherein the recombinase is inducible, and step (a) comprises inducing expression of the recombinase. In some such methods, the recombinase is inducible upon treatment with tamoxifen, and step (a) comprises treating the non-human animal with tamoxifen.

In some such methods, the control non-human animal is the non-human animal in step (a) prior to treatment with the recombinase. In some such methods, the control non-human animal is a second non-human animal comprising the reverse-conditional null endogenous Arsb gene, wherein the second non-human animal has not been treated with the recombinase. In some such methods, the second non-human animal is the same age as the non-human animal in step (a). In some such methods, step (b) further comprises assessing the phenotype of mucopolysaccharidosis VI after a defined time period post-treatment relative to a wild type non-human animal. Optionally, the wild type non-human animal is the same age as the non-human animal in step (b).

In some such methods, the defined time period post-treatment in step (b) is at least 1 week, at least 1 month, or at least 3 months after treatment. In some such methods, the defined time period post-treatment in step (b) is between about 1 month and about 3 months after treatment.

In some such methods, the phenotype is a skeletal phenotype. In some such methods, the phenotype comprises, relative to a wild type non-human animal: (i) increased accumulation of glycosaminoglycans in the liver; (ii) increased accumulation of glycosaminoglycans in the heart; (iii) increased accumulation of glycosaminoglycans in the kidney; (iv) decreased tibia length; (v) decreased spinal column length; (vi) increased tibial growth plate width; or (vii) decreased cranial length:width ratio. In some such methods, the phenotype comprises the decreased tibia length, the decreased spinal column length, the increased tibial growth plate width, or the decreased cranial length:width ratio. In some such methods, the phenotype comprises the decreased tibia length or the decreased spinal column length.

In another aspect, provided are methods of determining an optimal timeframe for treatment of a phenotype of mucopolysaccharidosis VI. Some such methods comprise: (a) performing any of the above methods of assessing reversibility of a phenotype of mucopolysaccharidosis VI a first time in a first non-human animal, wherein the first non-human animal is treated with the recombinase at a first time point after birth; (b) performing the method of step (a) a second time in a second non-human animal, wherein the second non-human animal is treated with the recombinase at a second time point after birth, wherein the second time point is different from the first time point; and (c) comparing the phenotype in step (a) with the phenotype in step (b) and selecting the time point resulting in the better amelioration of the phenotype.

In some such methods, the phenotype is a skeletal phenotype. In some such methods, the phenotype comprises, relative to a wild type non-human animal: (i) increased accumulation of glycosaminoglycans in the liver; (ii) increased accumulation of glycosaminoglycans in the heart; (iii) increased accumulation of glycosaminoglycans in the kidney; (iv) decreased tibia length; (v) decreased spinal column length; (vi) increased tibial growth plate width; or (vii) decreased cranial length:width ratio. In some such methods, the phenotype comprises the decreased tibia length, the decreased spinal column length, the increased tibial growth plate width, or the decreased cranial length:width ratio. In some such methods, the phenotype comprises the decreased tibia length or the decreased spinal column length.

In another aspect, provided are methods of assessing the reversibility of a phenotype of mucopolysaccharidosis VI by a combination therapy. Some such methods comprise: (a) performing any of the above methods of assessing reversibility of a phenotype of mucopolysaccharidosis VI a first time in a first non-human animal and a second time in a second non-human animal, wherein the first non-human animal is treated with the recombinase but not the candidate therapeutic agent, and wherein the second non-human animal is treated with the recombinase and the candidate therapeutic agent; and (b) comparing the phenotype in the first non-human animal with the phenotype in the second non-human animal to determine if the combination of the recombinase and the candidate therapeutic agent results in better amelioration of the phenotype.

In some such methods, the second non-human animal is treated with the recombinase and the candidate therapeutic agent simultaneously. In some such methods, the second non-human animal is treated with the recombinase and the candidate therapeutic agent sequentially. In some such methods, the candidate therapeutic agent is not a protein encoded by the endogenous Arsb gene or a nucleic acid encoding the protein.

In another aspect, provided are methods of assessing the efficacy of a candidate therapeutic agent for reversing a phenotype of mucopolysaccharidosis VI. Some such methods comprise: (a) performing any of the above methods of assessing reversibility of a phenotype of mucopolysaccharidosis VI a first time in a first non-human animal and a second time in a second non-human animal, wherein the first non-human animal is treated with the recombinase but not the candidate therapeutic agent, and wherein the second non-human animal is treated with the candidate therapeutic agent but not the recombinase; and (b) comparing the phenotype in the first non-human animal with the phenotype in the second non-human animal to determine if the recombinase results in better amelioration of the phenotype than the candidate therapeutic agent.

In some such methods, the recombinase and the candidate therapeutic agent are administered at the same selected time point after birth, and the phenotype is assessed after the same defined time period post-treatment in the first non-human animal and the second non-human animal. In some such methods, the candidate therapeutic agent is a protein encoded by the endogenous Arsb gene or a nucleic acid encoding the protein.

In another aspect, provided are methods of making any of the above non-human animals comprising in their genome a reverse-conditional null endogenous Arsb gene. Some such methods comprise: (a) introducing into a non-human animal host embryo a genetically modified non-human animal embryonic stem (ES) cell comprising in its genome the reverse-conditional null endogenous Arsb gene; and (b) gestating the non-human animal host embryo in a surrogate non-human animal mother, wherein the surrogate non-human animal mother produces an F0 progeny genetically modified non-human animal comprising the reverse-conditional null endogenous Arsb gene. Optionally, such methods further comprises modifying the non-human animal ES cell prior to step (a) to comprise in its genome the reverse-conditional null endogenous Arsb gene. Some such methods comprise gestating a genetically modified non-human animal host embryo in a non-human animal surrogate mother, wherein the genetically modified non-human animal host embryo comprises in its genome the reverse-conditional null endogenous Arsb gene, and wherein the surrogate non-human animal mother produces an F0 progeny genetically modified non-human animal comprising the reverse-conditional null endogenous Arsb gene. Optionally, such methods further comprise modifying a non-human animal one-cell stage embryo to generate the genetically modified non-human animal host embryo prior to the gestating step.

Non-human animals, non-human animal cells, and non-human animal genomes comprising a reverse-conditional null endogenous Idua gene are provided, as well as methods of making and using such non-human animals, non-human animal cells, and non-human animal genomes. Also provided are reverse-conditional null endogenous Idua genes, nucleic acids (e.g. isolated nucleic acids) comprising reverse-conditional null endogenous Idua genes, nuclease agents and/or targeting vectors for use in making reverse-conditional null endogenous Idua genes, and methods of making and using such reverse-conditional null endogenous Idua genes.

In one aspect, provided are non-human animals, non-human animal cells, and non-human animal genomes in their genome a reverse-conditional null endogenous Idua gene. In some such non-human animals, non-human animal cells, and non-human animal genomes, the reverse-conditional null endogenous Idua gene is a null endogenous Idua gene that is converted to a functional endogenous Idua gene upon treatment with a recombinase. In some such non-human animals, non-human animal cells, and non-human animal genomes, a critical region of the endogenous Idua gene is inverted and flanked by recombinase recognition sites in the reverse-conditional null endogenous Idua gene, wherein the recombinase recognition sites are oriented such that the critical region is reinverted upon treatment with the recombinase. In some such non-human animals, non-human animal cells, and non-human animal genomes, the critical region comprises a coding sequence of the endogenous Idua gene. In some such non-human animals, non-human animal cells, and non-human animal genomes, the critical region comprises a region from exon 2 to the stop codon of the endogenous Idua gene. In some such non-human animals, non-human animal cells, and non-human animal genomes, the recombinase is a Cre recombinase. In some such non-human animals, non-human animal cells, and non-human animal genomes, the recombinase recognition sites comprise a lox71 site and a lox66 site.

Some such non-human animals, non-human animal cells, and non-human animal genomes further comprise a genomically integrated recombinase expression cassette encoding the recombinase. In some such non-human animals, non-human animal cells, and non-human animal genomes, the genomically integrated recombinase expression cassette comprises a recombinase coding sequence operably linked to a tissue-specific promoter. In some such non-human animals, non-human animal cells, and non-human animal genomes, the recombinase is inducible. In some such non-human animals, non-human animal cells, and non-human animal genomes, the recombinase is inducible upon treatment with tamoxifen.

In some such non-human animals, non-human animal cells, and non-human animal genomes, the reverse-conditional null endogenous Idua gene comprises a selection cassette or a reporter gene flanked by recombinase recognition sites for a second recombinase. In some such non-human animals, non-human animal cells, and non-human animal genomes, the reverse-conditional null endogenous Idua gene comprises the selection cassette, wherein the selection cassette is a self-deleting selection cassette. Some such non-human animals, non-human animal cells, and non-human animal genomes do not comprise a selection cassette or a reporter gene.

In some such non-human animals, non-human animal cells, and non-human animal genomes, the non-human animal comprises the reverse-conditional null endogenous Idua gene in its germline. In some such non-human animals, non-human animal cells, and non-human animal genomes, the non-human animal is a mammal. In some such non-human animals, non-human animal cells, and non-human animal genomes, the non-human animal is a rodent. In some such non-human animals, non-human animal cells, and non-human animal genomes, the non-human animal is a mouse or a rat. In some such non-human animals, non-human animal cells, and non-human animal genomes, the non-human animal is the mouse.

In some such non-human animals, non-human animal cells, and non-human animal genomes, the non-human animal displays one or more phenotypes associated with mucopolysaccharidosis I. In some such non-human animals, non-human animal cells, and non-human animal genomes, the non-human animal displays one or more skeletal phenotypes associated with mucopolysaccharidosis I. In some such non-human animals, non-human animal cells, and non-human animal genomes, the non-human animal displays one or more neurological phenotypes associated with mucopolysaccharidosis I. In some such non-human animals, non-human animal cells, and non-human animal genomes, the non-human animal displays one or more skeletal phenotypes associated with mucopolysaccharidosis I and one or more neurological phenotypes associated with mucopolysaccharidosis I.

In some such non-human animals, non-human animal cells, and non-human animal genomes, a region comprising exon 2 to the stop codon of the endogenous Idua gene is inverted and flanked by recombinase recognition sites in the reverse-conditional null endogenous Idua gene, the recombinase recognition sites are oriented such that the region is reinverted upon treatment with the recombinase, the recombinase is a Cre recombinase, and the recombinase recognition sites comprise a lox71 site and a lox66 site, the non-human animal comprises the reverse-conditional null endogenous Idua gene in its germline, the non-human animal is a mouse, and the non-human animal displays one or more phenotypes associated with mucopolysaccharidosis I. In some such non-human animals, non-human animal cells, and non-human animal genomes, the non-human animal displays one or more skeletal phenotypes associated with mucopolysaccharidosis I. In some such non-human animals, non-human animal cells, and non-human animal genomes, the non-human animal displays one or more neurological phenotypes associated with mucopolysaccharidosis I. In some such non-human animals, non-human animal cells, and non-human animal genomes, the non-human animal displays one or more skeletal phenotypes associated with mucopolysaccharidosis I and one or more neurological phenotypes associated with mucopolysaccharidosis I. In some such non-human animals, non-human animal cells, and non-human animal genomes, the reverse-conditional null endogenous Idua gene comprises the sequence set forth in SEQ ID NO: 10 or 11. In some such non-human animals, non-human animal cells, and non-human animal genomes, the reverse-conditional null endogenous Idua gene comprises the sequence set forth in SEQ ID NO: 49. In some such non-human animals, non-human animal cells, and non-human animal genomes, upon treatment the reverse-conditional null endogenous Idua gene comprises the sequence set forth in SEQ ID NO: 12. In some such non-human animals, non-human animal cells, and non-human animal genomes, upon treatment the reverse-conditional null endogenous Idua gene comprises the sequence set forth in SEQ ID NO: 50.

In another aspect, provided are methods of assessing reversibility of a phenotype of mucopolysaccharidosis I. Some such methods comprise: (a) treating any of the above non-human animals comprising in their genome a reverse-conditional null endogenous Idua gene with the recombinase at a selected time point after birth or treating the non-human animal with a candidate therapeutic agent at the selected time point after birth; and (b) assessing the phenotype of mucopolysaccharidosis I after a defined time period post-treatment relative to a control non-human animal.

In some such methods, step (a) comprises treating the non-human animal with the candidate therapeutic agent at the selected time point after birth. Optionally, the candidate therapeutic agent is a protein encoded by the endogenous Idua gene or a nucleic acid encoding the protein. In some such methods, the control non-human animal is the non-human animal in step (a) prior to treatment with the candidate therapeutic agent. In some such methods, the control non-human animal is a second non-human animal comprising the reverse-conditional null endogenous Idua gene, wherein the second non-human animal has not been treated with the candidate therapeutic agent. Optionally, the second non-human animal is the same age as the non-human animal in step (a).

In some such methods, step (a) comprises treating the non-human animal with the recombinase at the selected time point after birth. In some such methods, the non-human animal further comprises a genomically integrated recombinase expression cassette encoding the recombinase, wherein the recombinase is inducible, and step (a) comprises inducing expression of the recombinase. In some such methods, the recombinase is inducible upon treatment with tamoxifen, and step (a) comprises treating the non-human animal with tamoxifen.

In some such methods, the control non-human animal is the non-human animal in step (a) prior to treatment with the recombinase. In some such methods, the control non-human animal is a second non-human animal comprising the reverse-conditional null endogenous Idua gene, wherein the second non-human animal has not been treated with the recombinase. In some such methods, the second non-human animal is the same age as the non-human animal in step (a). In some such methods, step (b) further comprises assessing the phenotype of mucopolysaccharidosis I after a defined time period post-treatment relative to a wild type non-human animal. Optionally, the wild type non-human animal is the same age as the non-human animal in step (b).

In some such methods, the defined time period post-treatment in step (b) is at least 1 week, at least 1 month, or at least 3 months after treatment. In some such methods, the defined time period post-treatment in step (b) is between about 1 month and about 3 months after treatment.

In some such methods, the phenotype is a skeletal phenotype. In some such methods, the phenotype is a neurological phenotype.

In another aspect, provided are methods of determining an optimal timeframe for treatment of a phenotype of mucopolysaccharidosis I. Some such methods comprise: (a) performing any of the above methods of assessing reversibility of a phenotype of mucopolysaccharidosis I a first time in a first non-human animal, wherein the first non-human animal is treated with the recombinase at a first time point after birth; (b) performing the method of step (a) a second time in a second non-human animal, wherein the second non-human animal is treated with the recombinase at a second time point after birth, wherein the second time point is different from the first time point; and (c) comparing the phenotype in step (a) with the phenotype in step (b) and selecting the time point resulting in the better amelioration of the phenotype. In some such methods, the phenotype is a skeletal phenotype. In some such methods, the phenotype is a neurological phenotype.

In another aspect, provided are methods of assessing the reversibility of a phenotype of mucopolysaccharidosis I by a combination therapy. Some such methods comprise: (a) performing any of the above methods of assessing reversibility of a phenotype of mucopolysaccharidosis I a first time in a first non-human animal and a second time in a second non-human animal, wherein the first non-human animal is treated with the recombinase but not the candidate therapeutic agent, and wherein the second non-human animal is treated with the recombinase and the candidate therapeutic agent; and (b) comparing the phenotype in the first non-human animal with the phenotype in the second non-human animal to determine if the combination of the recombinase and the candidate therapeutic agent results in better amelioration of the phenotype.

In some such methods, the second non-human animal is treated with the recombinase and the candidate therapeutic agent simultaneously. In some such methods, the second non-human animal is treated with the recombinase and the candidate therapeutic agent sequentially. In some such methods, the candidate therapeutic agent is not a protein encoded by the endogenous Idua gene or a nucleic acid encoding the protein.

In another aspect, provided are methods of assessing the efficacy of a candidate therapeutic agent for reversing a phenotype of mucopolysaccharidosis I. Some such methods comprise: (a) performing any of the above methods of assessing reversibility of a phenotype of mucopolysaccharidosis I a first time in a first non-human animal and a second time in a second non-human animal, wherein the first non-human animal is treated with the recombinase but not the candidate therapeutic agent, and wherein the second non-human animal is treated with the candidate therapeutic agent but not the recombinase; and (b) comparing the phenotype in the first non-human animal with the phenotype in the second non-human animal to determine if the recombinase results in better amelioration of the phenotype than the candidate therapeutic agent.

In some such methods, the recombinase and the candidate therapeutic agent are administered at the same selected time point after birth, and the phenotype is assessed after the same defined time period post-treatment in the first non-human animal and the second non-human animal. In some such methods, the candidate therapeutic agent is a protein encoded by the endogenous Idua gene or a nucleic acid encoding the protein.

In another aspect, provided are methods of making any of the above non-human animals comprising in their genome a reverse-conditional null endogenous Idua gene. Some such methods comprise: (a) introducing into a non-human animal host embryo a genetically modified non-human animal embryonic stem (ES) cell comprising in its genome the reverse-conditional null endogenous Idua gene; and (b) gestating the non-human animal host embryo in a surrogate non-human animal mother, wherein the surrogate non-human animal mother produces an F0 progeny genetically modified non-human animal comprising the reverse-conditional null endogenous Idua gene. Optionally, some such methods further comprise modifying the non-human animal ES cell prior to step (a) to comprise in its genome the reverse-conditional null endogenous Idua gene. Some such methods comprise gestating a genetically modified non-human animal host embryo in a non-human animal surrogate mother, wherein the genetically modified non-human animal host embryo comprises in its genome the reverse-conditional null endogenous Idua gene, and wherein the surrogate non-human animal mother produces an F0 progeny genetically modified non-human animal comprising the reverse-conditional null endogenous Idua gene. Optionally, some such methods further comprise modifying a non-human animal one-cell stage embryo to generate the genetically modified non-human animal host embryo prior to the gestating step.

### BRIEF DESCRIPTION OF THE FIGURES

FIG. 1 (not to scale) shows a schematic of the targeting scheme for generation of Arsb conditional by inversion (COIN) mice. The top portion of the figure shows the endogenous wild type mouse Arsb locus zoomed into exon 5. The bottom portion of the figure shows the targeted allele with or without the self-deleting selection cassette, and the cassette-deleted targeted allele after rescue by Cre recombinase.

FIG. 2 (not to scale) shows a schematic of the TAQMAN® assays for screening of the Arsb COIN allele. Gain-of-allele (GOA) assays include GOA2, GOA1, DreU, and Neo. Loss-of-allele (LOA) assays include mTU and mTD.

FIG. 3 shows skeletons from 18-19-week old female wild type and Arsb COIN (KO) mice.

FIG. 4 shows body weights of 18-19-week old female wild type and Arsb COIN (KO) mice. *p<0.05, **p<0.01, ***p<0.001, ****p<0.0001; ns=not significant.

FIG. 5 shows left tibia lengths in 18-19-week old female wild type and Arsb COIN (KO) mice. *p<0.05, **p<0.01, ***p<0.001, ****p<0.0001; ns=not significant.

FIG. 6 shows spinal column (L2-L6) lengths in 18-19-week old female wild type and Arsb COIN (KO) mice. *p<0.05, **p<0.01, ***p<0.001, ****p<0.0001; ns=not significant.

FIG. 7 shows cranial length:width ratios in 18-19-week old female wild type and Arsb COIN (KO) mice. *p<0.05, **p<0.01, ***p<0.001, ****p<0.0001; ns=not significant.

FIG. 8 shows glycosaminoglycan (GAG) accumulation in liver, heart, spinal cord, serum, spleen, and kidney in 18-19-week old female wild type and Arsb COIN (KO) mice. *p<0.05, **p<0.01, ***p<0.001, ****p<0.0001; ns=not significant.

FIG. 9 shows a schematic for an experimental design to assess phenotype reversibility at different time points using the Arsb COIN mice.

FIG. 10 shows residual glycosaminoglycans (GAGs) in liver, heart, and kidney in wild type adult mice (8-10 weeks), Arsb COIN (KO) adult mice, and Arsb COIN* (rescue) adult mice three months after tamoxifen dosing. *p<0.05, **p<0.01, ***p<0.001, ****p<0.0001; ns=not significant.

FIG. 11 shows left tibia in wild type adult mice, Arsb COIN (KO) adult mice, and Arsb COIN* (rescue) adult mice one month and three months after tamoxifen dosing. *p<0.05, **p<0.01, ***p<0.001, ****p<0.0001; ns=not significant.

FIG. 12 shows spinal column (L2-L6) lengths in wild type adult mice, Arsb COIN (KO) adult mice, and Arsb COIN* (rescue) adult mice one month and three months after tamoxifen dosing. *p<0.05, **p<0.01, ***p<0.001, ****p<0.0001; ns=not significant.

FIG. 13 shows cranial length:width ratios in wild type mice, Arsb COIN (KO) mice, and Arsb COIN* (rescue) mice one month and three months after tamoxifen dosing at 8-10 weeks afterbirth. *p<0.05, **p<0.01, ***p<0.001, ****p<0.0001; ns=not significant.

FIG. 14 shows tibial growth plate widths in wild type mice, Arsb COIN (KO) mice, and Arsb COIN* (rescue) mice one month and three months after tamoxifen dosing at 8-10 weeks afterbirth. *p<0.05, **p<0.01, ***p<0.001, ****p<0.0001; ns=not significant.

FIG. 15 shows trabecular bone parameters including trabecular bone volume, trabecular structural model index, trabecular thickness, trabecular separation, and trabecular number in wild type mice, Arsb COIN (KO) mice, and Arsb COIN* (rescue) mice one month after tamoxifen dosing at 8-10 weeks after birth. *p<0.05, **p<0.01, ***p<0.001, ****p<0.0001; ns=not significant. The structural model index is an estimate of the shape of trabeculae (how rod- or plate-like). A value of zero represents a perfect plate, while three represents a cylindrical rod. A perfect sphere would have a value of four. The geometry of trabeculae has an effect on how they behave as load bearing structures and their contribution to whole bone strength. The trabecular thickness is the mean thickness of the trabeculae, assessed using direct 3D methods (mm). The trabecular separation is the mean distance between the trabeculae, assessed using direct 3D methods (mm). The trabecular number is the measure of the average number of trabeculae per unit length (1/mm).

FIG. 16 shows residual glycosaminoglycans (GAGs) in liver, heart, and kidney in wild type mice, Arsb COIN (KO) mice, and Arsb COIN* (rescue) mice three months after tamoxifen dosing at postnatal day 21. *p<0.05, **p<0.01, ***p<0.001, ****p<0.0001; ns=not significant.

FIG. 17 shows left tibia lengths in wild type mice, Arsb COIN (KO) mice, and Arsb COIN* (rescue) mice one month and three months after tamoxifen dosing at postnatal day 21. *p<0.05, **p<0.01, ***p<0.001, ****p<0.0001; ns=not significant.

FIG. 18 shows spinal column (L2-L6) lengths in wild type mice, Arsb COIN (KO) mice, and Arsb COIN* (rescue) mice one month and three months after tamoxifen dosing at postnatal day 21. *p<0.05, **p<0.01, ***p<0.001, ****p<0.0001; ns=not significant.

FIG. 19 shows cranial length:width ratios in wild type mice, Arsb COIN (KO) mice, and Arsb COIN* (rescue) mice one month and three months after tamoxifen dosing at postnatal day 21. *p<0.05, **p<0.01, ***p<0.001, ****p<0.0001; ns=not significant.

FIG. 20 shows tibial growth plate widths in wild type mice, Arsb COIN (KO) mice, and Arsb COIN* (rescue) mice one month and three months after tamoxifen dosing at postnatal day 21. *p<0.05, **p<0.01, ***p<0.001, ****p<0.0001; ns=not significant.

FIG. 21 shows residual glycosaminoglycans (GAGs) in liver, heart, and kidney in wild type mice, Arsb COIN (KO) mice, and Arsb COIN* (rescue) mice one month after tamoxifen dosing at postnatal day 7. *p<0.05, **p<0.01, ***p<0.001, ****p<0.0001; ns=not significant.

FIG. 22 shows residual glycosaminoglycans (GAGs) in liver, heart, and kidney in wild type mice, Arsb COIN (KO) mice, and Arsb COIN* (rescue) mice three months after tamoxifen dosing at postnatal day 7. *p<0.05, **p<0.01, ***p<0.001, ****p<0.0001; ns=not significant.

FIG. 23 shows skeletons in wild type mice, Arsb COIN (KO) mice, and Arsb COIN* (rescue) mice three months after tamoxifen dosing at postnatal day 7.

FIG. 24 shows left tibia lengths in wild type mice, Arsb COIN (KO) mice, and Arsb COIN* (rescue) mice one month and three months after tamoxifen dosing at postnatal day 7. *p<0.05, **p<0.01, ***p<0.001, ****p<0.0001; ns=not significant.

FIG. 25 shows left spinal column (L2-L6) lengths in wild type mice, Arsb COIN (KO) mice, and Arsb COIN* (rescue) mice one month and three months after tamoxifen dosing at postnatal day 7. *p<0.05, **p<0.01, ***p<0.001, ****p<0.0001; ns=not significant.

FIG. 26 shows cranial length:width ratios in wild type mice, Arsb COIN (KO) mice, and Arsb COIN* (rescue) mice one month and three months after tamoxifen dosing at postnatal day 7. *p<0.05, **p<0.01, ***p<0.001, ****p<0.0001; ns=not significant.

FIG. 27 shows tibial growth plate widths in wild type mice, Arsb COIN (KO) mice, and Arsb COIN* (rescue) mice three months after tamoxifen dosing at postnatal day 7. *p<0.05, **p<0.01, ***p<0.001, ****p<0.0001; ns=not significant.

FIG. 28 shows alcian blue staining images of tibial growth plate widths in wild type mice, Arsb COIN (KO) mice, and Arsb COIN* (rescue) mice one month and three months after tamoxifen dosing at postnatal day 7.

FIG. 29 shows a schematic for an experimental design to assess phenotype reversibility at different time points using the Arsb COIN mice compared to enzyme replacement therapy (ERT).

FIG. 30 shows residual glycosaminoglycans (GAGs) in liver, heart, and kidney in wild type adult mice (6-8 weeks), Arsb COIN (KO) adult mice, Arsb COIN (KO) adult mice+ERT, and COIN* (rescue) adult mice one month after tamoxifen dosing. *p<0.05, **p<0.01, ***p<0.001, ****p<0.0001; ns=not significant.

FIG. 31 shows left tibia lengths (left panel) and spinal column (L2-L6) lengths (right panel) in wild type adult mice, Arsb COIN (KO) adult mice, Arsb COIN (KO) adult mice +ERT, and Arsb COIN* (rescue) adult mice one month after tamoxifen dosing. *p<0.05, **p<0.01, ***p<0.001, ****p<0.0001; ns=not significant.

FIG. 32 shows images of tibial growth plates (alcian blue stain) in wild type adult mice, Arsb COIN (KO) adult mice, Arsb COIN (KO) adult mice+ERT, and Arsb COIN* (rescue) adult mice one month after tamoxifen dosing.

FIG. 33 shows growth plate widths in wild type adult mice, Arsb COIN (KO) adult mice, Arsb COIN (KO) adult mice+ERT, and Arsb COIN* (rescue) adult mice one month after tamoxifen dosing. *p<0.05, **p<0.01, ***p<0.001, ****p<0.0001; ns=not significant.

FIGS. 34A-34C show Arsb COIN allele recombination rates and transcript abundance in peripheral organs when tamoxifen is delivered at P56-P70 in liver (FIG. 34A), heart (FIG. 34B), and kidney (FIG. 34C). Percentage recombination is assessed as ΔCt of Arsb Lox71 specific sequence compared to a serial standard, with GAPDH as a reference. Transcript abundance is assessed as ΔΔCt against WT samples as references. Data are shown as the mean±SD (n=4 to 9). Two-way ANOVAs were performed for comparison, as indicated.

FIGS. 35A-35C show restoration of Arsb expression at P7 improves femoral bone mass. FIG. 35A shows high resolution microCT images of femoral distal metaphyses at 3 months post-tamoxifen treatment. FIG. 35B shows quantification of trabecular readouts.

FIG. 35C shows quantification of cortical readouts. Data are shown as the mean±SD (n=8 to 17). Repeated measures one-way ANOVAs were performed, as indicated. *p<0.05, **p<0.01, ***p<0.001, ****p<0.0001.

FIG. 36 (not to scale) shows a schematic of the targeting scheme for generation of Idua conditional by inversion (COIN) mice. The top portion of the figure shows the endogenous wild type mouse Idua locus. The bottom portion of the figure shows the targeted allele with or without the self-deleting selection cassette, and the cassette-deleted targeted allele after rescue by Cre recombinase.

FIG. 37 (not to scale) shows a schematic of the TAQMAN® assays for screening of the Arsb COIN allele. Loss-of-allele (LOA) assays include mTU and mTD. Retention assays include mretU and mretD.

FIG. 38 shows left tibia lengths in 14-week old male and female wild type and Idua COIN (KO) mice. *p<0.05, **p<0.01, ***p<0.001, ****p<0.0001; ns=not significant.

FIG. 39 shows spinal column (L2-L6) lengths in 14-week old male and female wild type and Idua COIN (KO) mice. *p<0.05, **p<0.01, ***p<0.001, ****p<0.0001; ns=not significant.

FIG. 40 shows cranial length:width ratios in 14-week old male and female wild type and Idua COIN (KO) mice. *p<0.05, **p<0.01, ***p<0.001, ****p<0.0001; ns=not significant.

FIG. 41 shows last lumbar ribcage width in 14-week old male and female wild type and Idua COIN (KO) mice. *p<0.05, **p<0.01, ***p<0.001, ****p<0.0001; ns=not significant.

FIG. 42 shows left zygomatic arch length:width ratios in 14-week old male and female wild type and Idua COIN (KO) mice. *p<0.05, **p<0.01, ***p<0.001, ****p<0.0001; ns=not significant.

FIG. 43 shows a schematic for an experimental design to assess phenotype reversibility at different time points using the Idua COIN mice.

FIG. 44 shows residual glycosaminoglycans (GAGs) in liver, heart, kidney, spleen, cerebellum, and cerebrum in wild type adult mice (6-8 weeks), Idua COIN (KO) adult mice, COIN* (rescue) adult mice, and heterozygous (WT/COIN) adult mice one month after tamoxifen dosing. *p<0.05, **p<0.01, ***p<0.001, ****p<0.0001; ns=not significant.

### DEFINITIONS

The terms “protein,” “polypeptide,” and “peptide,” used interchangeably herein, include polymeric forms of amino acids of any length, including coded and non-coded amino acids and chemically or biochemically modified or derivatized amino acids. The terms also include polymers that have been modified, such as polypeptides having modified peptide backbones. The term “domain” refers to any part of a protein or polypeptide having a particular function or structure.

Proteins are said to have an “N-terminus” (amino-terminus) and a “C-terminus” (carboxy-terminus or carboxyl-terminus). The term “N-terminus” relates to the start of a protein or polypeptide, terminated by an amino acid with a free amine group (—NH2). The term “C-terminus” relates to the end of an amino acid chain (protein or polypeptide), terminated by a free carboxyl group (—COOH).

The terms “nucleic acid” and “polynucleotide,” used interchangeably herein, include polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

Nucleic acids are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. An end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. A nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements.

The term “genomically integrated” refers to a nucleic acid that has been introduced into a cell such that the nucleotide sequence integrates into the genome of the cell. Any protocol may be used for the stable incorporation of a nucleic acid into the genome of a cell.

The term “targeting vector” refers to a recombinant nucleic acid that can be introduced by homologous recombination, non-homologous-end-joining-mediated ligation, or any other means of recombination to a target position in the genome of a cell.

The term “viral vector” refers to a recombinant nucleic acid that includes at least one element of viral origin and includes elements sufficient for or permissive of packaging into a viral vector particle. The vector and/or particle can be utilized for the purpose of transferring DNA, RNA, or other nucleic acids into cells in vitro, ex vivo, or in vivo. Numerous forms of viral vectors are known.

The term “isolated” with respect to cells, tissues (e.g., liver samples), lipid droplets, proteins, and nucleic acids includes cells, tissues (e.g., liver samples), lipid droplets, proteins, and nucleic acids that are relatively purified with respect to other bacterial, viral, cellular, or other components that may normally be present in situ, up to and including a substantially pure preparation of the cells, tissues (e.g., liver samples), lipid droplets, proteins, and nucleic acids. The term “isolated” also includes cells, tissues (e.g., liver samples), lipid droplets, proteins, and nucleic acids that have no naturally occurring counterpart, have been chemically synthesized and are thus substantially uncontaminated by other cells, tissues (e.g., liver samples), lipid droplets, proteins, and nucleic acids, or has been separated or purified from most other components (e.g., cellular components or organism components) with which they are naturally accompanied (e.g., other cellular proteins, nucleic acids, or cellular or extracellular components).

The term “wild type” includes entities having a structure and/or activity as found in a normal (as contrasted with mutant, diseased, altered, or so forth) state or context. Wild type genes and polypeptides often exist in multiple different forms (e.g., alleles).

The term “endogenous sequence” refers to a nucleic acid sequence that occurs naturally within a rat cell or rat. For example, an endogenous Arsb sequence of a mouse refers to a native Arsb sequence that naturally occurs at the Arsb locus in the mouse.

“Exogenous” molecules or sequences include molecules or sequences that are not normally present in a cell in that form. Normal presence includes presence with respect to the particular developmental stage and environmental conditions of the cell. An exogenous molecule or sequence, for example, can include a mutated version of a corresponding endogenous sequence within the cell, such as a humanized version of the endogenous sequence, or can include a sequence corresponding to an endogenous sequence within the cell but in a different form (i.e., not within a chromosome). In contrast, endogenous molecules or sequences include molecules or sequences that are normally present in that form in a particular cell at a particular developmental stage under particular environmental conditions.

The term “heterologous” when used in the context of a nucleic acid or a protein indicates that the nucleic acid or protein comprises at least two segments that do not naturally occur together in the same molecule. For example, the term “heterologous,” when used with reference to segments of a nucleic acid or segments of a protein, indicates that the nucleic acid or protein comprises two or more sub-sequences that are not found in the same relationship to each other (e.g., joined together) in nature. As one example, a “heterologous” region of a nucleic acid vector is a segment of nucleic acid within or attached to another nucleic acid molecule that is not found in association with the other molecule in nature. For example, a heterologous region of a nucleic acid vector could include a coding sequence flanked by sequences not found in association with the coding sequence in nature. Likewise, a “heterologous” region of a protein is a segment of amino acids within or attached to another peptide molecule that is not found in association with the other peptide molecule in nature (e.g., a fusion protein, or a protein with a tag). Similarly, a nucleic acid or protein can comprise a heterologous label or a heterologous secretion or localization sequence.

“Codon optimization” takes advantage of the degeneracy of codons, as exhibited by the multiplicity of three-base pair codon combinations that specify an amino acid, and generally includes a process of modifying a nucleic acid sequence for enhanced expression in particular host cells by replacing at least one codon of the native sequence with a codon that is more frequently or most frequently used in the genes of the host cell while maintaining the native amino acid sequence. For example, a nucleic acid encoding an ARSB protein can be modified to substitute codons having a higher frequency of usage in a given prokaryotic or eukaryotic cell, including a bacterial cell, a yeast cell, a human cell, a non-human cell, a mammalian cell, a rodent cell, a mouse cell, a rat cell, a hamster cell, or any other host cell, as compared to the naturally occurring nucleic acid sequence. Codon usage tables are readily available, for example, at the “Codon Usage Database.” These tables can be adapted in a number of ways. See Nakamura et al. (2000) Nucleic Acids Res. 28(1):292, herein incorporated by reference in its entirety for all purposes. Computer algorithms for codon optimization of a particular sequence for expression in a particular host are also available (see, e.g., Gene Forge).

The term “locus” refers to a specific location of a gene (or significant sequence), DNA sequence, polypeptide-encoding sequence, or position on a chromosome of the genome of an organism. For example, an “Arsb locus” may refer to the specific location of an Arsb gene, Arsb DNA sequence, ARSB-encoding sequence, or Arsb position on a chromosome of the genome of an organism that has been identified as to where such a sequence resides. An “Arsb locus” may comprise a regulatory element of an Arsb gene, including, for example, an enhancer, a promoter, 5′ and/or 3′ untranslated region (UTR), or a combination thereof.

The term “gene” refers to DNA sequences in a chromosome that may contain, if naturally present, at least one coding and at least one non-coding region. The DNA sequence in a chromosome that codes for a product (e.g., but not limited to, an RNA product and/or a polypeptide product) can include the coding region interrupted with non-coding introns and sequence located adjacent to the coding region on both the 5′ and 3′ ends such that the gene corresponds to the full-length mRNA (including the 5′ and 3′ untranslated sequences). Additionally, other non-coding sequences including regulatory sequences (e.g., but not limited to, promoters, enhancers, and transcription factor binding sites), polyadenylation signals, internal ribosome entry sites, silencers, insulating sequence, and matrix attachment regions may be present in a gene. These sequences may be close to the coding region of the gene (e.g., but not limited to, within 10 kb) or at distant sites, and they influence the level or rate of transcription and translation of the gene.

The term “allele” refers to a variant form of a gene. Some genes have a variety of different forms, which are located at the same position, or genetic locus, on a chromosome. A diploid organism has two alleles at each genetic locus. Each pair of alleles represents the genotype of a specific genetic locus. Genotypes are described as homozygous if there are two identical alleles at a particular locus and as heterozygous if the two alleles differ.

A “promoter” is a regulatory region of DNA usually comprising a TATA box capable of directing RNA polymerase II to initiate RNA synthesis at the appropriate transcription initiation site for a particular polynucleotide sequence. In some cases, a promoter may additionally comprise other regions which influence the transcription initiation rate. The promoter sequences disclosed herein modulate transcription of an operably linked polynucleotide. A promoter can be active in one or more of the cell types disclosed herein (e.g., a mouse cell, a rat cell, a pluripotent cell, a one-cell stage embryo, a differentiated cell, or a combination thereof). A promoter can be, for example, a constitutively active promoter, a conditional promoter, an inducible promoter, a temporally restricted promoter (e.g., a developmentally regulated promoter), or a spatially restricted promoter (e.g., a cell-specific or tissue-specific promoter). Examples of promoters can be found, for example, in WO 2013/176772, herein incorporated by reference in its entirety for all purposes.

A constitutive promoter is one that is active in all tissues or particular tissues at all developing stages. Examples of constitutive promoters include the human cytomegalovirus immediate early (hCMV), mouse cytomegalovirus immediate early (mCMV), human elongation factor 1 alpha (hEF1α), mouse elongation factor 1 alpha (mEF1α), mouse phosphoglycerate kinase (PGK), chicken beta actin hybrid (CAG or CBh), SV40 early, and beta 2 tubulin promoters.

Examples of inducible promoters include, for example, chemically regulated promoters and physically-regulated promoters. Chemically regulated promoters include, for example, alcohol-regulated promoters (e.g., an alcohol dehydrogenase (alcA) gene promoter), tetracycline-regulated promoters (e.g., a tetracycline-responsive promoter, a tetracycline operator sequence (tetO), a tet-On promoter, or a tet-Off promoter), steroid regulated promoters (e.g., a rat glucocorticoid receptor, a promoter of an estrogen receptor, or a promoter of an ecdysone receptor), or metal-regulated promoters (e.g., a metalloprotein promoter). Physically regulated promoters include, for example temperature-regulated promoters (e.g., a heat shock promoter) and light-regulated promoters (e.g., a light-inducible promoter or a light-repressible promoter).

Tissue-specific promoters can be, for example, neuron-specific promoters, glia-specific promoters, muscle cell-specific promoters, heart cell-specific promoters, kidney cell-specific promoters, bone cell-specific promoters, endothelial cell-specific promoters, or immune cell-specific promoters (e.g., a B cell promoter or a T cell promoter).

Developmentally regulated promoters include, for example, promoters active only during an embryonic stage of development, or only in an adult cell.

“Operable linkage” or being “operably linked” includes juxtaposition of two or more components (e.g., a promoter and another sequence element) such that both components function normally and allow the possibility that at least one of the components can mediate a function that is exerted upon at least one of the other components. For example, a promoter can be operably linked to a coding sequence if the promoter controls the level of transcription of the coding sequence in response to the presence or absence of one or more transcriptional regulatory factors. Operable linkage can include such sequences being contiguous with each other or acting in trans (e.g., a regulatory sequence can act at a distance to control transcription of the coding sequence).

The methods and compositions provided herein employ a variety of different components. Some components throughout the description can have active variants and fragments. The term “functional” refers to the innate ability of a protein or nucleic acid (or a fragment or variant thereof) to exhibit a biological activity or function. The biological functions of functional fragments or variants may be the same or may in fact be changed (e.g., with respect to their specificity or selectivity or efficacy) in comparison to the original molecule, but with retention of the molecule's basic biological function.

The term “variant” refers to a nucleotide sequence differing from the sequence most prevalent in a population (e.g., by one nucleotide) or a protein sequence different from the sequence most prevalent in a population (e.g., by one amino acid).

The term “fragment,” when referring to a protein, means a protein that is shorter or has fewer amino acids than the full-length protein. The term “fragment,” when referring to a nucleic acid, means a nucleic acid that is shorter or has fewer nucleotides than the full-length nucleic acid. A protein fragment can be, for example, an N-terminal fragment (i.e., removal of a portion of the C-terminal end of the protein), a C-terminal fragment (i.e., removal of a portion of the N-terminal end of the protein), or an internal fragment (i.e., removal of a portion of each of the N-terminal and C-terminal ends of the protein). A nucleic acid fragment can be, for example, a 5′ fragment (i.e., removal of a portion of the 3′ end of the nucleic acid), a 3′ fragment (i.e., removal of a portion of the 5′ end of the nucleic acid), or an internal fragment (i.e., removal of a portion each of the 5′ and 3′ ends of the nucleic acid).

“Sequence identity” or “identity” in the context of two polynucleotides or polypeptide sequences refers to the residues in the two sequences that are the same when aligned for maximum correspondence over a specified comparison window. When percentage of sequence identity is used in reference to proteins, residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

“Percentage of sequence identity” includes the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, sequence identity/similarity values include the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. “Equivalent program” includes any sequence comparison program that, for any two sequences in question, generates an alignment having identical nucleotide or amino acid residue matches and an identical percent sequence identity when compared to the corresponding alignment generated by GAP Version 10.

The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, or leucine for another non-polar residue. Likewise, examples of conservative substitutions include the substitution of one polar (hydrophilic) residue for another such as between arginine and lysine, between glutamine and asparagine, or between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, or methionine for a polar (hydrophilic) residue such as cysteine, glutamine, glutamic acid or lysine and/or a polar residue for a non-polar residue. Typical amino acid categorizations are summarized below.

TABLE 1
Amino Acid Categorizations.

| | | | | | |
|---|---|---|---|---|---|
| Alanine | Ala | A | Nonpolar | Neutral | 1.8 |
| Arginine | Arg | R | Polar | Positive | −4.5 |
| Asparagine | Asn | N | Polar | Neutral | −3.5 |
| Aspartic acid | Asp | D | Polar | Negative | −3.5 |
| Cysteine | Cys | C | Nonpolar | Neutral | 2.5 |
| Glutamic acid | Glu | E | Polar | Negative | −3.5 |
| Glutamine | Gln | Q | Polar | Neutral | −3.5 |
| Glycine | Gly | G | Nonpolar | Neutral | −0.4 |
| Histidine | His | H | Polar | Positive | −3.2 |
| Isoleucine | Ile | I | Nonpolar | Neutral | 4.5 |
| Leucine | Leu | L | Nonpolar | Neutral | 3.8 |
| Lysine | Lys | K | Polar | Positive | −3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | −1.6 |
| Serine | Ser | S | Polar | Neutral | −0.8 |
| Threonine | Thr | T | Polar | Neutral | −0.7 |
| Tryptophan | Trp | W | Nonpolar | Neutral | −0.9 |
| Tyrosine | Tyr | Y | Polar | Neutral | −1.3 |
| Valine | Val | V | Nonpolar | Neutral | 4.2 |

A “homologous” sequence (e.g., nucleic acid sequence) includes a sequence that is either identical or substantially similar to a known reference sequence, such that it is, for example, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the known reference sequence. Homologous sequences can include, for example, orthologous sequence and paralogous sequences. Homologous genes, for example, typically descend from a common ancestral DNA sequence, either through a speciation event (orthologous genes) or a genetic duplication event (paralogous genes). “Orthologous” genes include genes in different species that evolved from a common ancestral gene by speciation. Orthologs typically retain the same function in the course of evolution. “Paralogous” genes include genes related by duplication within a genome. Paralogs can evolve new functions in the course of evolution.

The term “in vitro” includes artificial environments and to processes or reactions that occur within an artificial environment (e.g., a test tube or an isolated cell or cell line). The term “in vivo” includes natural environments (e.g., an organism or body or a cell or tissue within an organism or body) and to processes or reactions that occur within a natural environment. The term “ex vivo” includes cells that have been removed from the body of an individual and processes or reactions that occur within such cells.

The term “reporter gene” refers to a nucleic acid having a sequence encoding a gene product (typically an enzyme) that is easily and quantifiably assayed when a construct comprising the reporter gene sequence operably linked to a heterologous promoter and/or enhancer element is introduced into cells containing (or which can be made to contain) the factors necessary for the activation of the promoter and/or enhancer elements. Examples of reporter genes include, but are not limited, to genes encoding beta-galactosidase (lacZ), the bacterial chloramphenicol acetyltransferase (cat) genes, firefly luciferase genes, genes encoding beta-glucuronidase (GUS), and genes encoding fluorescent proteins. A “reporter protein” refers to a protein encoded by a reporter gene.

The term “fluorescent reporter protein” as used herein means a reporter protein that is detectable based on fluorescence wherein the fluorescence may be either from the reporter protein directly, activity of the reporter protein on a fluorogenic substrate, or a protein with affinity for binding to a fluorescent tagged compound. Examples of fluorescent proteins include green fluorescent proteins (e.g., GFP, GFP-2, tagGFP, turboGFP, eGFP, Emerald, Azami Green, Monomeric Azami Green, CopGFP, AceGFP, and ZsGreen1), yellow fluorescent proteins (e.g., YFP, eYFP, Citrine, Venus, YPet, PhiYFP, and ZsYellow1), blue fluorescent proteins (e.g., BFP, eBFP, eBFP2, Azurite, mKalama1, GFPuv, Sapphire, and T-sapphire), cyan fluorescent proteins (e.g., CFP, eCFP, Cerulean, CyPet, AmCyan1, and Midoriishi-Cyan), red fluorescent proteins (e.g., RFP, mKate, mKate2, mPlum, DsRed monomer, mCherry, mRFP1, DsRed-Express, DsRed2, DsRed-Monomer, HcRed-Tandem, HcRed1, AsRed2, eqFP611, mRaspberry, mStrawberry, and Jred), orange fluorescent proteins (e.g., mOrange, mKO, Kusabira-Orange, Monomeric Kusabira-Orange, mTangerine, and tdTomato), and any other suitable fluorescent protein whose presence in cells can be detected by flow cytometry methods.

Repair in response to double-strand breaks (DSBs) occurs principally through two conserved DNA repair pathways: homologous recombination (HR) and non-homologous end joining (NHEJ). See Kasparek & Humphrey (2011) Semin. Cell Dev. Biol. 22(8):886-897, herein incorporated by reference in its entirety for all purposes. Likewise, repair of a target nucleic acid mediated by an exogenous donor nucleic acid can include any process of exchange of genetic information between the two polynucleotides.

The term “recombination” includes any process of exchange of genetic information between two polynucleotides and can occur by any mechanism. Recombination can occur via homology directed repair (HDR) or homologous recombination (HR). HDR or HR includes a form of nucleic acid repair that can require nucleotide sequence homology, uses a “donor” molecule as a template for repair of a “target” molecule (i.e., the one that experienced the double-strand break), and leads to transfer of genetic information from the donor to target. Without wishing to be bound by any particular theory, such transfer can involve mismatch correction of heteroduplex DNA that forms between the broken target and the donor, and/or synthesis-dependent strand annealing, in which the donor is used to resynthesize genetic information that will become part of the target, and/or related processes. In some cases, the donor polynucleotide, a portion of the donor polynucleotide, a copy of the donor polynucleotide, or a portion of a copy of the donor polynucleotide integrates into the target DNA. See Wang et al. (2013) Cell 153:910-918; Mandalos et al. (2012) PLoS ONE 7:e45768:1-9; and Wang et al. (2013) Nat. Biotechnol. 31:530-532, each of which is herein incorporated by reference in its entirety for all purposes.

Non-homologous end joining (NHEJ) includes the repair of double-strand breaks in a nucleic acid by direct ligation of the break ends to one another or to an exogenous sequence without the need for a homologous template. Ligation of non-contiguous sequences by NHEJ can often result in deletions, insertions, or translocations near the site of the double-strand break. For example, NHEJ can also result in the targeted integration of an exogenous donor nucleic acid through direct ligation of the break ends with the ends of the exogenous donor nucleic acid (i.e., NHEJ-based capture). Such NHEJ-mediated targeted integration can be preferred for insertion of an exogenous donor nucleic acid when homology directed repair (HDR) pathways are not readily usable (e.g., in non-dividing cells, primary cells, and cells which perform homology-based DNA repair poorly). In addition, in contrast to homology-directed repair, knowledge concerning large regions of sequence identity flanking the cleavage site is not needed, which can be beneficial when attempting targeted insertion into organisms that have genomes for which there is limited knowledge of the genomic sequence. The integration can proceed via ligation of blunt ends between the exogenous donor nucleic acid and the cleaved genomic sequence, or via ligation of sticky ends (i.e., having 5′ or 3′ overhangs) using an exogenous donor nucleic acid that is flanked by overhangs that are compatible with those generated by a nuclease agent in the cleaved genomic sequence. See, e.g., US 2011/020722, WO 2014/033644, WO 2014/089290, and Maresca et al. (2013) Genome Res. 23(3):539-546, each of which is herein incorporated by reference in its entirety for all purposes. If blunt ends are ligated, target and/or donor resection may be needed to generation regions of microhomology needed for fragment joining, which may create unwanted alterations in the target sequence.

Compositions or methods “comprising” or “including” one or more recited elements may include other elements not specifically recited. For example, a composition that “comprises” or “includes” a protein may contain the protein alone or in combination with other ingredients. The transitional phrase “consisting essentially of” means that the scope of a claim is to be interpreted to encompass the specified elements recited in the claim and those that do not materially affect the basic and novel characteristic(s) of the claimed invention. Thus, the term “consisting essentially of” when used in a claim of this invention is not intended to be interpreted to be equivalent to “comprising.”

“Optional” or “optionally” means that the subsequently described event or circumstance may or may not occur and that the description includes instances in which the event or circumstance occurs and instances in which the event or circumstance does not.

Designation of a range of values includes all integers within or defining the range, and all subranges defined by integers within the range.

Unless otherwise apparent from the context, the term “about” encompasses values±5 of a stated value.

The term “and/or” refers to and encompasses any and all possible combinations of one or more of the associated listed items, as well as the lack of combinations when interpreted in the alternative (“or”).

The term “or” refers to any one member of a particular list.

The singular forms of the articles “a,” “an,” and “the” include plural references unless the context clearly dictates otherwise. For example, the term “a protein” or “at least one protein” can include a plurality of proteins, including mixtures thereof.

Statistically significant means p≤0.05.

### DETAILED DESCRIPTION

### I. Overview

Disclosed herein are non-human animal genomes, non-human animal cells, and non-human animals comprising a reverse-conditional null endogenous lysosomal storage disease gene and methods of making and using such non-human animal cells and non-human animals. Also disclosed herein are methods of using the non-human animals for assessing reversibility of a phenotype of the lysosomal storage disease or for determining an optimal timeframe for treatment of a phenotype of the lysosomal storage disease. Also disclosed herein are non-human animal reverse-conditional null endogenous lysosomal storage disease genes and nuclease agents and targeting vectors for use in making a non-human animal reverse-conditional null endogenous lysosomal storage disease gene.

Mucopolysaccharidoses (MPS) are a subset of lysosomal diseases that are caused by a recessive deficiency in enzymes required for the degradation of glycosaminoglycans (GAGs). GAGs are functionally diverse, but play critical roles in tissue maturation, extracellular matrix (ECM) formation and function, and the development of the skeleton. Seven types of MPS are characterized, and across types there exists significant overlap in clinical phenotypes associated with disease. Nearly all MPS subtypes include a spectrum of dysostosis multiplex, or progressive skeletal dysplasia. Mucopolysaccharidosis VI (MPS VI), also known as Maroteaux-Lamy Syndrome, is a lysosomal disease resulting from impaired function of the arylsulfatase B (ARSB) protein. This impairment causes aberrant accumulation of dermatan sulfate, a glycosaminoglycan (GAG) abundant in growth plates, cartilage, and extracellular matrix. While clinical presentation is variable in terms of age at first symptom manifestation and disease severity, MPS VI classically presents at early ages and significantly impacts the skeleton. Current treatment guidelines recommend enzyme replacement therapy (ERT), which is known to provide incomplete or ineffective recovery from the skeletal manifestations of disease. We can postulate this may be due to the inability of the exogenous enzyme to reach affected cells, or that disease may not be reversible at the time therapy is delivered. To date, no models of disease exist that separate treatment efficacy from disease reversibility. Similar issues exist with models for other mucopolysaccharidosis diseases (e.g., MPS I) and other lysosomal storage diseases. The existing models do not resolve reversibility or treatment efficiency questions, as they look at delivery of a therapeutic at a single (or very few) timepoints, and are restricted by limitations on uptake of the therapeutic. To date, no model exists where efficacy of ERT and reversibility of the phenotype can be explored independently.

In order to separate reversibility of phenotype from questions about recombinant enzyme delivery, we have developed novel models employing a conditional inversion method. To this end, we have developed lysosomal storage disease mouse models comprising a reverse-conditional null endogenous lysosomal storage disease gene that can turn back on endogenous expression of the missing lysosomal storage disease gene enzyme at different points during disease progression to properly assess reversibility of disease phenotypes separate from efficacy of a particular treatment. In other words, the models described herein can inform us regarding the maximal possible correction we can expect to achieve for phenotypes associated with lysosomal storage diseases if we can develop an optimized therapeutic. In one specific example, exon 5 of the murine Arsb gene is inverted, preventing any functional expression of ARSB. It is also flanked by lox sites (lox66 and lox71 sites), which allow for the exon to be flipped back to the native orientation in the presence of Cre recombinase. In another specific example, a region from intron 3 through the 3′ UTR of the Idua gene was inverted in the mouse Idua locus, preventing any functional expression of IDUA. It is also flanked by lox sites (lox66 and lox71 sites), which allow for the region to be flipped back to the native orientation in the presence of Cre recombinase. Unlike classic floxed-stop conditional-on allele approaches, where wild type might result from readthrough past the floxed-stop cassette and RNA splicing, in this COIN-based approach no functional message can be made. Introduction of a controllable Cre recombinase (e.g., Cre-ER^{T2}) allows for restoration of the wild-type orientation of the exon at specific timepoints. Therefore, we can restore native ARSB expression or IDUA expression from the endogenous locus at various developmental timepoints, allowing for reinstitution of protein in the correct cells and at natural levels.

### II. Non-Human Animals Comprising a Reverse-Conditional Null Endogenous Lysosomal Storage Disease Gene

The non-human animal genomes, non-human animal cells, and non-human animals described herein comprise a reverse-conditional null endogenous lysosomal storage disease gene.

### A. Lysosomal Storage Disease Genes

The non-human animal genomes, non-human animal cells, and non-human animals described herein comprise a reverse-conditional null endogenous lysosomal storage disease gene (e.g., a reverse-conditional null endogenous mouse lysosomal storage disease gene or a reverse-conditional null endogenous rat lysosomal storage disease gene). An endogenous lysosomal storage disease gene means a lysosomal storage disease gene at the endogenous genomic locus for that lysosomal storage disease gene. In some cases, an endogenous lysosomal storage disease gene in a non-human animal can be an endogenous non-human animal lysosomal storage disease gene, meaning that it has not been humanized (i.e., a fully non-human animal lysosomal storage disease gene). For example, an endogenous lysosomal storage disease gene in a mouse can be an endogenous mouse lysosomal storage disease gene at the endogenous mouse locus for that lysosomal storage disease gene (i.e., a fully mouse lysosomal storage disease gene). In other cases, an endogenous lysosomal storage gene at the endogenous locus can be humanized (e.g., a segment of the endogenous lysosomal storage disease gene locus has been deleted and replaced with a corresponding human lysosomal storage gene sequence). A lysosomal storage disease gene is a gene that encodes a protein that is deficient in a lysosomal storage disease. Lysosomal storage diseases are a group of over 70 rare inherited metabolic disorders that result from defects in lysosomal function. Lysosomal storage diseases are caused by lysosomal dysfunction, usually as a consequence of deficiency of a single enzyme required for the metabolism of lipids, glycoproteins, or mucopolysaccharides. Most of lysosomal storage diseases are autosomal recessively inherited (e.g., Niemann-Pick disease, type C), but a few are X-linked recessively inherited (e.g., Fabry disease and Hunter syndrome (MPS II)). The pathogeneses of the diseases are ascribed to the buildup of incomplete degradation products in the lysosome, usually due to loss of protein function. Lysosomal storage diseases are caused by loss-of-function or attenuating variants in the proteins whose normal function is to degrade or coordinate degradation of lysosomal contents. The proteins affiliated with lysosomal storage diseases include enzymes, receptors and other transmembrane proteins (e.g., NPC1), post-translational modifying proteins (e.g., sulfatase), membrane transport proteins, and non-enzymatic cofactors and other soluble proteins (e.g., GM2 ganglioside activator).

Lysosomal storage diseases are a class of rare diseases that affect the degradation of myriad substrates in the lysosome. Those substrates include sphingolipids, mucopolysaccharides, glycoproteins, glycogen, and oligosaccharides, which can accumulate in the cells of those with disease leading to cell death. Organs affected by lysosomal storage diseases include the central nervous system (CNS), the peripheral nervous system (PNS), lungs, liver, bone, skeletal and cardiac muscle, and the reticuloendothelial system.

Lysosomal storage diseases include sphingolipidoses, a mucopolysaccharidoses, and glycogen storage diseases. In some embodiments, the lysosomal storage disease is any one of Fabry disease, Gaucher disease type I, Gaucher disease type II, Gaucher disease type III, Niemann-Pick disease type A, Niemann-Pick disease type BGM1-gangliosidosis, Sandhoff disease, Tay-Sachs disease, GM2-activator deficiency, GM3-gangliosidosis, metachromatic leukodystrophy, sphingolipid-activator deficiency, Scheie disease, Hurler-Scheie disease, Hurler disease, Hunter disease, Sanfilippo A, Sanfilippo B, Sanfilippo C, Sanfilippo D, Morquio syndrome A, Morquio syndrome B, Maroteaux-Lamy disease, Sly disease, MPS IX, and Pompe disease. Enzymes (which include proteins that are not per se catalytic) associated with lysosomal storage diseases include for example any and all hydrolases, α-galactosidase, β-galactosidase, α-glucosidase, β-glucosidase, saposin-C activator, ceramidase, sphingomyelinase, β-hexosaminidase, GM2 activator, GM3 synthase, arylsulfatase, sphingolipid activator, α-iduronidase, iduronidase-2-sulfatase, heparin N-sulfatase, N-acetyl-α-glucosaminidase, α-glucosamide N-acetyltransferase, N-acetylglucosamine-6-sulfatase, N-acetylgalactosamine-6-sulfate sulfatase, N-acetylgalactosamine-4-sulfatase, β-glucuronidase, hyaluronidase, and the like.

The nature of the molecular lesion affects the severity of the disease in many cases. Complete loss-of-function tends to be associated with prenatal or neonatal onset, and involves severe symptoms; partial loss-of-function is associated with milder (relatively) and later-onset disease. Generally, only a small percentage of activity needs to be restored to have to correct metabolic defects in deficient cells. Table 2 lists some of the more common lysosomal storage diseases and their associated loss-of-function proteins.

TABLE 2
Lysosomal Storage Diseases.

| Class | Disease | Involved Enzyme/Protein |
|---|---|---|
| Sphingolipidoses | Fabry disease | α-Galactosidase A |
| | Farber lipogranulomatosis | Ceramidase |
| | Gaucher disease type I | β-Glucosidase |
| | Gaucher disease types II and III | Saposin-C activator |
| | Niemann-Pick diseases types A and B | Sphingomyelinase |
| | GM1-gangliosidosis | β-Galactosidase |
| | GM2-gangliosidosis (Sandhoff) | β-Hexosaminidase A and B |
| | GM2-gangliosidosis (Tay-Sachs) | β-Hexosaminidase A |
| | GM2-gangliosidosis (GM2-activator | GM2-activator protein |
| | deficiency) | |
| | GM3-gangliosidosis | GM3 synthase |
| | Metachromatic leukodystrophy | Arylsulfatase A |
| | Sphingolipid-activator deficiency | Sphingolipid activator |
| Mucopoly- | MPS I (Scheie, Hurler-Scheie, | α-Iduronidase |
| saccharidoses | and Hurler disease) | |
| | MPS II (Hunter) | Iduronidase-2-sulphatase |
| | MPS IIIA (Sanfilippo A) | Heparan N-sulphatase |
| | MPS IIIB (Sanfilippo B) | N-acetyl-α-glucosaminidase |
| | MPS IIIC (Sanfilippo C) | Acetyl-CoA; α-glucosamide |
| | | N-acetyltransferase |
| | MPS IIID (Sanfilippo D) | N-acetylglucosamine-6- sulphatase |
| | MPS IVA (Morquio syndrome A) | N-acetylgalactosamine-6- sulphate |
| | | sulphatase |
| | MPS IVB (Morquio syndrome B) | β-Galactosidase |
| | MPS VI (Maroteaux-Lamy) | N-acetylgalactosamine-4- |
| | | sulphatase (arylsulphatase B) |
| | MPS VII (Sly disease) | β-Glucuronidase |
| | MPS IX | Hylauronidase |
| Glycogen | Pompe (glycogen storage | α-Glucosidase 2 |
| storage disease | disease type II) | |
| Lipid metabolism | Lysosomal acid lipase deficiency | Lysosomal acid lipase |
| | (LAL-D; Wolman disease) | |

Lysosomal storage diseases can be categorized according to the type of product that accumulates within the defective lysosome. Sphingolipidoses are a class of diseases that affect the metabolism of sphingolipids, which are lipids containing fatty acids linked to aliphatic amino alcohols. The accumulated products of sphingolipidoses include gangliosides (e.g., Tay-Sachs disease), glycolipids (e.g., Fabry's disease), and glucocerebrosides (e.g., Gaucher's disease).

Mucopolysaccharidoses are a group of diseases that affect the metabolism of glycosaminoglycans (GAGS or mucopolysaccharides), which are long unbranched chains of repeating disaccharides that help build bone, cartilage, tendons, corneas, skin and connective tissue. The accumulated products of mucopolysaccharidoses include heparan sulfate, dermatan sulfate, keratin sulfate, various forms of chondroitin sulfate, and hyaluronic acid. For example, Morquio syndrome A is due to a defect in the lysosomal enzyme galactose-6-sulfate sulfatase, which results in the lysosomal accumulation of keratin sulfate and chondroitin 6-sulfate.

Glycogen storage diseases result from a cell's inability to metabolize (make or break-down) glycogen. Glycogen metabolism is moderated by various enzymes or other proteins including glucose-6-phosphatase, acid alpha-glucosidase, glycogen de-branching enzyme, glycogen branching enzyme, muscle glycogen phosphorylase, liver glycogen phosphorylase, muscle phosphofructokinase, phosphorylase kinase, glucose transporter, aldolase A, beta-enolase, and glycogen synthase. An exemplar lysosomal storage/glycogen storage disease is Pompe disease, in which defective acid alpha-glucosidase causes glycogen to accumulate in lysosomes. Symptoms include hepatomegaly, muscle weakness, heart failure, and in the case of the infantile variant, death by age two.

In a particular example, a lysosomal storage disease gene can be a mucopolysaccharidosis gene. A mucopolysaccharidosis is a gene that encodes a protein that is deficient in a mucopolysaccharidosis (MPS). In one specific example, the MPS is MPS VI, and the gene is Arsb. In another specific example, the MPS is MPS I, and the gene is Idua.

Mucopolysaccharidoses are a group of metabolic disorders caused by the absence or malfunctioning of lysosomal enzymes needed to break down molecules called glycosaminoglycans (GAGs). These long chains of sugar carbohydrates occur within the cells that help build bone, cartilage, tendons, corneas, skin and connective tissue. GAGs (formerly called mucopolysaccharides) are also found in the fluids that lubricate joints.

Individuals with mucopolysaccharidosis either do not produce enough of one of the eleven enzymes required to break down these sugar chains into simpler molecules, or they produce enzymes that do not work properly. Over time, these GAGs collect in the cells, blood and connective tissues. The result is permanent, progressive cellular damage which affects appearance, physical abilities, organ and system functioning.

The mucopolysaccharidoses share many clinical features but have varying degrees of severity. These features may not be apparent at birth but progress as storage of GAGs affects bone, skeletal structure, connective tissues, and organs. Neurological complications may include damage to neurons (which send and receive signals throughout the body) as well as pain and impaired motor function. This results from compression of nerves or nerve roots in the spinal cord or in the peripheral nervous system, the part of the nervous system that connects the brain and spinal cord to sensory organs such as the eyes and to other organs, muscles, and tissues throughout the body.

Individuals with MPS disorders share many similar symptoms such as multiple organ involvement, distinctive coarse facial features, and abnormalities of the skeleton especially joint problems. Additional findings include short stature, heart abnormalities, breathing irregularities, liver and spleen enlargement (hepatosplenomegaly), and/or neurological abnormalities. The severity of the different MPS disorders varies greatly among affected individuals, even among those with the same type of MPS and even among individuals of the same family. Different mucopolysaccharidoses are set forth in Table 3.

TABLE 3
Mucopolysaccharidoses.

| MPS | Common name; | | | Accumulated | |
|---|---|---|---|---|---|
| Type | Other names | Gene | Deficient enzyme | products | Symptoms |
| MPS | Hurler syndrome | IDUA | α-L-iduronidase | Heparan | Intellectual |
| IH | | | | sulfate; | disability, |
| MPS | Hurler-Scheie syndrome | | | Dermatan | micrognathia, coarse |
| IH/S | | | | sulfate | facial features, |
| MPS | Scheie syndrome; Formerly: | | | | macroglossia, retinal |
| IS | Mucopolysaccharidosis type | | | | degeneration, |
| | V | | | | corneal clouding, |
| | | | | | cardiomyopathy, |
| | | | | | hepatosplenomegaly |
| MPS | Hunter syndrome | IDS | Iduronate sulfatase | Heparan | Intellectual |
| II | | | | sulfate; | disability (similar, |
| | | | | Dermatan | but milder, symptoms |
| | | | | sulfate | to MPS I). This type |
| | | | | | exceptionally has X- |
| | | | | | linked recessive |
| | | | | | inheritance |
| MPS | Sanfilippo syndrome A; | SGSH | Heparan sulfamidase | Heparan | Developmental delay, |
| IIIA | Sulfamidase deficiency | | | sulfate | severe hyperactivity, |
| MPS | Sanfilippo syndrome B; | NAGLU | N- | | spasticity, motor |
| IIIB | NAGLU deficiency | | acetylglucosaminidase | | dysfunction, death |
| MPS | Sanfilippo syndrome C | HGSNAT | Heparan-α- | | by the second decade |
| IIIC | | | glucosaminide N- | | |
| | | | acetyltransferase | | |
| MPS | Sanfilippo syndrome D | GNS | N-acetylglucosamine | | |
| IIID | | | 6-sulfatase | | |
| MPS | Morquio syndrome A | GALNS | Galactose-6-sulfate | Keratan | Severe skeletal |
| IVA | | | sulfatase | sulfate; | dysplasia, short |
| | | | | Chondroitin | stature, motor |
| | | | | 6-sulfate | dysfunction |
| MPS | Morquio syndrome B | GLB1 | β-galactosidase | Keratan | |
| IVB | | | | sulfate | |
| MPS | Maroteaux-Lamy syndrome; | ARSB | N- | Dermatan | Severe skeletal |
| VI | ARSB deficiency | | acetylgalactosamine- | sulfate | dysplasia, short |
| | | | 4-sulfatase | | stature, motor |
| | | | | | dysfunction, |
| | | | | | kyphosis, heart |
| | | | | | defects |
| MPS | Sly syndrome; GUSB | GUSB | β-glucuronidase | Heparan | Hepatomegaly, |
| VII | deficiency | | | sulfate; | skeletal dysplasia, |
| | | | | Dermatan | short stature, corneal |
| | | | | sulfate; | clouding, |
| | | | | Chondroitin | developmental delay |
| | | | | 4,6-sulfate | |
| MPS | Natowicz syndrome; | HYAL1 | Hyaluronidase | Hyaluronic | Nodular soft-tissue |
| IX | Hyaluronidase deficiency | | | acid | masses around joints, |
| | | | | | episodes of painful |
| | | | | | swelling of the masses, |
| | | | | | short-term pain, mild |
| | | | | | facial changes, short |
| | | | | | stature, normal joint |
| | | | | | movement, normal |
| | | | | | intelligence |

In a specific example, the MPS is MPS VI. Maroteaux-Lamy syndrome (mucopolysaccharidosis type VI; MPS VI) is characterized by a deficiency of the enzyme N-acetylgalactosamine-4-sulfatase, resulting in accumulation of dermatan sulfate. This form of MPS varies greatly among affected individuals. Some affected individuals only experience a few mild symptoms, other develop a more severe form of the disorder. Possible symptoms of Maroteaux-Lamy syndrome include coarse facial features, umbilical hernia, a prominent breastbone (pectus carinatum), joint contractures, clouding of the corneas, and an abnormal enlargement of the liver and/or spleen (heptasplenomegaly). Skeletal malformations and heart disease may occur in individuals with this form of MPS. In most cases, intelligence is normal. In 2005, the U.S. Food and Drug Administration (FDA) approved galsulfase (Naglazyme) for the treatment of MPS VI, also known as Maroteaux-Lamy syndrome. Characterization of MPS VI patients and their symptoms has been more extensive in recent years. There are over 200 documented mutations of the ARSB gene, along with burgeoning evidence that genotype-phenotype correlations can be clinically useful. Nonsense mutations, indels, and early frameshifts have been shown to correlate with rapidly progressing and/or severe forms of disease. However, extensive clinical characterization of individual mutations, especially missense mutations, are problematic as most are novel and rare. Across mutation types, symptoms include a spectrum of heart abnormalities, corneal clouding, organomegaly, craniofacial deformities, scoliosis or kyphosis, and shortened stature. Enzyme replacement therapy (ERT) using recombinant human ARSB, galsulfase, has been approved and available for non-curative treatment of MPS VI since 2005. Recommendations include a weekly, four hour intravenous administration with antihistamine and/or antipyretic cocktail prior to each transfusion to control for infusion adverse reactions. In the clinic, ERT quickly and effectively reduces urinary GAG and hepatosplenomegaly. It is known, however, that there is limited efficacy for the spectrum of skeletal disease, with a sporadically reported, modest improvement in shoulder flexion.

In a specific example, the MPS is MPS I. Hurler syndrome (mucopolysaccharidosis type 1-H; MPS 1-H) is the most severe form of mucopolysaccharidosis. It is characterized by a deficiency of the enzyme alpha-L-iduronidase, which results in an accumulation of dermatan and heparan sulfates. Symptoms of the disorder first become evident at six months to two years of age. Affected infants may experience developmental delays, recurrent urinary and upper respiratory tract infections, noisy breathing and persistent nasal discharge. Additional physical problems may include clouding of the cornea of the eye, an unusually large tongue, severe deformity of the spine, and joint stiffness. Mental development begins to regress at about the age of two. Scheie syndrome (mucopolysaccharidosis type I-S; MPS 1-S) is the mildest form of mucopolysaccharidosis. As in Hurler syndrome, individuals with Scheie syndrome have a deficiency of the enzyme alpha-L-iduronidase. However, in Scheie syndrome the deficiency is specific for accumulation of dermatan sulfate. Individuals with Scheie syndrome have normal intelligence, height, and life expectancy. Symptoms include stiff joints, carpal tunnel syndrome, backward flow of blood into the heart (aortic regurgitation), and clouding of the cornea that may result in the loss of visual acuity. The onset of symptoms in individuals with Scheie syndrome usually occurs around the age of five. Hurler-Scheie syndrome (mucopolysaccharidosis type I-H/S; MPS-IH/S) is an extremely rare disorder that refers to individuals who have a less severe form of Hurler syndrome, but a more severe form than Scheie syndrome. Like Scheie syndrome, affected individuals have a deficiency of the alpha-L-iduronidase specific for accumulation of dermatan sulfate. Hurler-Scheie syndrome is not as severe as Hurler syndrome, but more severe than Scheie syndrome. Affected individuals may develop coarse facial features, joint stiffness, short stature, clouding of the corneas, abnormally enlarged liver and/spleen (hepatosplenomegaly), and skeletal and cardiac abnormalities. Intelligence may be normal or mild to moderate intellectual disability may develop. Symptoms usually become apparent between three and six years of age. In 2003, The FDA approved laronidase (Aldurazyme) as a treatment for MPS I. Specifically, this enzyme replacement therapy is approved for treating patients with the Hurler and Hurler-Scheie forms of MPS I and those with the Scheie form who exhibit moderate to severe symptoms. Clinical trials using laronidase show limited improvement on skeletal phenotypes and little to no improvement on cognitive phenotypes in MPS I. See, e.g., Dornelles et al. (2017) PLoS One 12(8):e0184065 and Chen et al. (2020) Mol. Genet. Metab. 129(2):80-90, each of which is herein incorporated by reference in its entirety for all purposes.

In a specific example, the lysosomal storage disease gene is an Arsb gene. Arsb (also known as Asl or Asl-s) encodes arylsulfatase B (also known as ARSB, ASB, N-acetylgalactosamine-4-sulfatase, or G4S). ARSB is a lysosomal enzyme required for the catalysis of the sulfate moiety on the terminal ring at the non-reducing end of the GAG, dermatan sulfate (DS), also known as chondroitin sulfate B. In addition to key roles in maintaining ECM stability by direct interaction with structural proteins like collagen and fibronectin, DS serves as a binding factor for adhesins and defensins expressed by specific pathogens, and plays a role in chemokine and cytokine binding. In some instances, this may be accomplished by the establishment of a solubility gradient as GAG structures coordinate water extremely well, and can have an indirect effect on ligand-receptor binding. In the context of the skeletal phenotype exhibited by MPS VI patients, DS-specific roles in cartilage and bone development may play a role in disease progression. It is known that multiple proteoglycans, such as decorin, epiphycans, and biglycans, are directly modified by or are composed of DS structures, and that such modifications have been known to effect growth factor signaling integral for cell proliferation and differentiation. In addition, loss of or impaired function of enzymes in mucopolysaccharidoses primarily cause accumulation of substrates in lysosomes, with downstream effects linked to lysosomal dysfunction, such as impaired vesicular trafficking, autophagy, and ion homeostasis which likely contribute to clinical manifestations. Pathogenic Arsb gene variants cause MPS VI. ARSB is involved in the breakdown of large sugar molecules called glycosaminoglycans (GAGs). Mutations in the Arsb gene reduce or completely eliminate the function of ARSB. The lack of ARSB activity leads to the accumulation of GAGs within cells, specifically inside the lysosomes.

Mouse Arsb maps to 13 C3; 13 47.88 cM on chromosome 13 (NCBI RefSeq Gene ID 11881; Assembly GRCm39 (GCF_000001635.27); location NC_000079.7 (93908187 . . . 94079524)). The gene has been reported to have 8 exons. The mouse ARSB protein has been assigned UniProt Accession No. P50429. The sequence for the mouse ARSB protein (NCBI Accession No. NP_033842.3) is set forth in SEQ ID NO: 1. An mRNA (cDNA) encoding the canonical isoform is assigned NCBI Accession No. NM_009712.3 and is set forth in SEQ ID NO: 2. An exemplary coding sequence (CDS) for the canonical isoform is set forth in SEQ ID NO: 3 (CCDS ID CCDS36749.1). The full-length mouse ARSB protein set forth in SEQ ID NO: 1 has 534 amino acids, including a signal peptide (amino acids 1-39) and an intracellular domain (amino acids 40-534). Delineations between these domains are as designated in UniProt. Reference to mouse ARSB includes the canonical isoform set forth in SEQ ID NO: 1 as well as all allelic forms and isoforms. Any other forms of mouse ARSB have amino acids numbered for maximal alignment with the canonical isoform, aligned amino acids being designated the same number. Rat ARSB protein has been assigned UniProt Accession No. P50430 and NCBI GeneID 25227. Human ARSB protein has been assigned UniProt Accession No. P15848 and NCBI GeneID 411.

In another specific example, the lysosomal storage disease gene is an Idua gene. Idua encodes alpha-L-iduronidase (also known as IDUA). Pathogenic Idua gene variants cause MPS I. IDUA is involved in the breakdown of large sugar molecules called glycosaminoglycans (GAGs). Mutations in the Idua gene reduce or completely eliminate the function of IDUA. The lack of IDUA activity leads to the accumulation of GAGs within cells, specifically inside the lysosomes.

Mouse Idua maps to 5 F; 5 53.24 cM on chromosome 5 (NCBI RefSeq Gene ID 15932; Assembly GRCm39 (GCF_000001635.27); location NC_000071.7 (108808197 . . . 108833312)). The gene has been reported to have 15 exons. The mouse IDUA protein has been assigned UniProt Accession No. P48441. The sequence for the mouse IDUA protein (NCBI Accession No. NP_032351.2) is set forth in SEQ ID NO: 4. An mRNA (cDNA) encoding the canonical isoform is assigned NCBI Accession No. NM_008325.4 and is set forth in SEQ ID NO: 5. An exemplary coding sequence (CDS) for the canonical isoform is set forth in SEQ ID NO: 6 (CCDS ID CCDS19516.2). The full-length mouse IDUA protein set forth in SEQ ID NO: 4 has 643 amino acids, including a signal peptide (amino acids 1-25) and an intracellular domain (amino acids 26-643). Delineations between these domains are as designated in UniProt. Reference to mouse IDUA includes the canonical isoform set forth in SEQ ID NO: 4 as well as all allelic forms and isoforms. Any other forms of mouse IDUA have amino acids numbered for maximal alignment with the canonical isoform, aligned amino acids being designated the same number. Rat ARSB protein has been assigned UniProt Accession No. D3ZE16 and NCBI GeneID 360904. Human IDUA protein has been assigned UniProt Accession No. P35475 and NCBI GeneID 3425.

### B. Reverse-Conditional Null Lysosomal Storage Disease Genes

Disclosed herein are reverse-conditional null endogenous lysosomal storage disease genes (e.g., reverse-conditional null endogenous mucopolysaccharidosis genes, reverse-conditional null endogenous Arsb genes, or reverse-conditional null endogenous Idua genes). Such reverse-conditional null endogenous lysosomal storage disease genes are null endogenous lysosomal storage disease genes that are converted to a functional endogenous lysosomal storage disease gene in response to a signal. For example, such reverse-conditional null endogenous lysosomal storage disease genes can be a null endogenous lysosomal storage disease gene that is converted to a functional endogenous lysosomal storage disease gene upon treatment with a recombinase.

A reverse-conditional null gene or allele is a gene or allele that is null but can be converted to a functional gene or allele in response to a signal (i.e., the gene or allele is inactive until the signal, in which case the gene or allele's expression and function return to normal compared to an unmodified gene or allele). For example, a reverse-conditional null gene or allele can be a gene or allele that is null but can be converted to a functional gene or allele upon treatment with a recombinase. A null gene or allele is a nonfunctional gene or allele caused by a genetic mutation. Such mutations can cause a complete lack of production of the associated gene product or a product that does not function properly; in either case, the gene or allele may be considered nonfunctional. A null allele cannot be distinguished from deletion of the entire locus solely from phenotypic observation. In a specific example, the reverse-conditional null gene or allele is inactivated by inversion of a critical region of the gene or allele (e.g., a critical region of its coding sequence) so that it is an orientation opposite to the gene's or allele's direction of transcription. The reverse-conditional null gene or allele can then be converted to a functionally wild type allele by inversion (i.e., reinversion) of the inverted critical region (i.e., inversion back to the same orientation as the gene's or allele's direction of transcription). Thus, reverse-conditional null genes or alleles can be used to explore the effect of restoration of gene function at different times and in different tissues.

In a specific example, a critical region of the endogenous lysosomal storage disease gene is inverted and flanked by recombinase recognition sites in the reverse-conditional null endogenous lysosomal storage disease gene, wherein the recombinase recognition sites are oriented such that the critical region is inverted (i.e., reinverted) upon treatment with a recombinase. Site-specific recombinases include enzymes that can facilitate recombination between recombinase recognition sites, where the two recombination sites are physically separated within a single nucleic acid or on separate nucleic acids. Recombinases are able to either delete sequences between the site-specific recombination sites if the sites are oriented in the same direction with respect to one another or invert the sequences between the site-specific recombination sites if the sites are oriented in opposite directions with respect to one another. Examples of recombinases include Cre, Flp, and Dre recombinases. One example of a Cre recombinase gene is Crei, in which two exons encoding the Cre recombinase are separated by an intron to prevent its expression in a prokaryotic cell. Such recombinases can further comprise a nuclear localization signal to facilitate localization to the nucleus (e.g., NLS-Crei). Recombinase recognition sites include nucleotide sequences that are recognized by a site-specific recombinase and can serve as a substrate for a recombination event. Examples of recombinase recognition sites include FRT, FRT11, FRT71, attp, att, rox, and lox sites such as loxP, lox511, lox2272, lox66, lox71, loxM2, and lox5171.

In one example, the recombinase is a Cre recombinase, and the site-specific recombination sites are Lox sites. For example, the site-specific recombination sites can comprise a lox66 site and a lox71 site (e.g., lox66 flanking the 5′ end and lox71 flanking the 3′ end). Cre-mediated inversion can convert the lox66-lox71 pair into an lox72-loxP pair, which does not support reinversion. In another specific example, the recombinase is a Flp recombinase, and the site-specific recombination sites are FRT sites. In another specific example, the recombinase is a Dre recombinase, and the site-specific recombination sites are rox sites.

A critical region of a gene or allele is a region of a gene or allele that is required for expression or activity of the gene or allele. For example, a critical region of a gene or allele can comprise a critical region of its coding sequence. In one example, a gene or allele without the critical region (or with the critical region inverted) will not be expressed. In another example, a gene or allele without the critical region (or with the critical region inverted) will be expressed, but the resulting gene product will be nonfunctional. In a specific example, the reverse-conditional null endogenous lysosomal storage disease gene is a reverse-conditional null endogenous Arsb gene, and the critical region comprises exon 5 of the endogenous Arsb gene (e.g., endogenous mouse Arsb gene). In another specific example, the reverse-conditional null endogenous lysosomal storage disease gene is a reverse-conditional null endogenous Idua gene, and the critical region comprises exon 2 to the stop codon of the endogenous Idua gene (e.g., endogenous mouse Idua gene).

The non-human animal genomes, non-human animal cells, and non-human animals described herein can further comprise a recombinase expression cassette that drives expression of the site-specific recombinase. The recombinase expression cassette can be genomically integrated. The recombinase gene in a recombinase expression cassette can be operably linked to any suitable promoter. Examples of promoters are disclosed elsewhere herein. For example, the promoter can be a tissue-specific promoter or a developmental-stage-specific promoter. The advantage provided by such promoters is that recombinase expression can be activated selectively in a desired tissue or only at a desired developmental stage. Such promoters are well-known.

Recombinase expression cassettes or recombinases can also be inducible, wherein expression of the recombinase or recombinase activity is induced in response to a particular signal. For example, the recombinase can be inducible upon treatment with tamoxifen. This offers both cell type-specific and temporal control of conditional gene inactivation. A specific example is a tamoxifen-inducible Cre recombinase, which can utilize a tamoxifen-inducible Cre-estrogen receptor (ER) fusion protein. Cre is fused with the ligand-binding domain of a mutated ligand-binding domain of ER, which does not bind to estrogen but binds to tamoxifen with high affinity. Cre-ER proteins are sequestered in the cytoplasm via association with the HSP90 chaperone. Upon addition of tamoxifen, tamoxifen-bound Cre-ER dissociates from HSP90, translocates into the nucleus, and carries out site-specific recombination between flanking lox sites. In a specific example, the Cre recombinase is Cre-ER^{T2}, in which additional mutations have been engineered into the ER ligand-binding domain to bind tamoxifen with a higher affinity.

In a specific example, a recombinase expression cassette is integrated into the genome at a safe harbor locus (e.g., a Rosa26 locus). Safe harbor loci include chromosomal loci where transgenes or other exogenous nucleic acid inserts can be stably and reliably expressed in all tissues of interest without overtly altering cell behavior or phenotype (i.e., without any deleterious effects on the host cell). See, e.g., Sadelain et al. (2012) Nat. Rev. Cancer 12:51-58, herein incorporated by reference in its entirety for all purposes. For example, the safe harbor locus can be one in which expression of the inserted gene sequence is not perturbed by any read-through expression from neighboring genes. For example, safe harbor loci can include chromosomal loci where exogenous DNA can integrate and function in a predictable manner without adversely affecting endogenous gene structure or expression. Safe harbor loci can include extragenic regions or intragenic regions such as, for example, loci within genes that are non-essential, dispensable, or able to be disrupted without overt phenotypic consequences. For example, the Rosa26 locus and its equivalent in humans offer an open chromatin configuration in all tissues and is ubiquitously expressed during embryonic development and in adults. See, e.g., Zambrowicz et al. (1997) Proc. Natl. Acad. Sci. USA 94:3789-3794, herein incorporated by reference in its entirety for all purposes. In addition, the Rosa26 locus can be targeted with high efficiency, and disruption of the Rosa26 gene produces no overt phenotype. Other examples of safe harbor loci include CCR5, HPRT, AAVS1, and albumin. See, e.g., U.S. Pat. Nos. 7,888,121; 7,972,854; 7,914,796; 7,951,925; 8,110,379; 8,409,861; 8,586,526; and US Patent Publication Nos. 2003/0232410; 2005/0208489; 2005/0026157; 2006/0063231; 2008/0159996; 2010/00218264; 2012/0017290; 2011/0265198; 2013/0137104; 2013/0122591; 2013/0177983; 2013/0177960; and 2013/0122591, each of which is herein incorporated by reference in its entirety for all purposes. In a specific example, a recombinase expression cassette is integrated into the genome at a Rosa26 locus. In another specific example, a Cre-ERT^{T2} expression cassette is integrated into the genome at a Rosa26 locus.

Optionally, a reverse-conditional null endogenous lysosomal storage disease gene can comprise other elements. Examples of such elements can include selection cassettes, reporter genes, recombinase recognition sites, or other elements. Alternatively, the reverse-conditional null endogenous lysosomal storage disease gene can lack other elements (e.g., can lack a selection marker or selection cassette). Examples of suitable reporter genes and reporter proteins are disclosed elsewhere herein. Examples of suitable selection markers include neomycin phosphotransferase (neor), hygromycin B phosphotransferase (hygr), puromycin-N-acetyltransferase (puror), blasticidin S deaminase (bsrr), xanthine/guanine phosphoribosyl transferase (gpt), and herpes simplex virus thymidine kinase (HSV-k). Examples of recombinases include Cre, Flp, and Dre recombinases. One example of a Cre recombinase gene is Crei, in which two exons encoding the Cre recombinase are separated by an intron to prevent its expression in a prokaryotic cell. Such recombinases can further comprise a nuclear localization signal to facilitate localization to the nucleus (e.g., NLS-Crei). Recombinase recognition sites include nucleotide sequences that are recognized by a site-specific recombinase and can serve as a substrate for a recombination event. Examples of recombinase recognition sites include FRT, FRT11, FRT71, attp, att, rox, and lox sites such as loxP, lox511, lox2272, lox66, lox71, loxM2, and lox5171.

Other elements such as reporter genes or selection cassettes can be self-deleting cassettes flanked by recombinase recognition sites. See, e.g., U.S. Pat. No. 8,697,851 and US 2013/0312129, each of which is herein incorporated by reference in its entirety for all purposes. The recombinase recognition sites in the self-deleting cassette can be different from the recombinase recognition sites used for rescue of the reverse-conditional null endogenous lysosomal storage disease gene. For example, the recombinase recognition sites used for rescue of the reverse-conditional null endogenous lysosomal storage disease gene can be lox sites (e.g., lox71 and lox66), and the recombinase recognition sites flanking the self-deleting cassette can be rox sites. As an example, the self-deleting cassette can comprise a Cre recombinase gene or a Dre recombinase gene operably linked to a mouse Prm1 promoter and a neomycin resistance gene operably linked to a human ubiquitin promoter. By employing the Prm1 promoter, the self-deleting cassette can be deleted specifically in male germ cells of F0 animals. The polynucleotide encoding the selection marker can be operably linked to a promoter active in a cell being targeted. Examples of promoters are described elsewhere herein. As another specific example, a self-deleting selection cassette can comprise a hygromycin resistance gene coding sequence operably linked to one or more promoters (e.g., both human ubiquitin and EM7 promoters) followed by a polyadenylation signal, followed by a Cre coding sequence or Dre coding sequence operably linked to one or more promoters (e.g., an mPrm1 promoter), followed by another polyadenylation signal, wherein the entire cassette is flanked by loxP sites or rox sites.

The reverse-conditional null endogenous lysosomal storage disease gene can be a multifunctional allele as described in US 2011/0104799, herein incorporated by reference in its entirety for all purposes. For example, the conditional allele can comprise: (a) an actuating sequence in sense orientation with respect to transcription of a target gene; (b) a drug selection cassette (DSC) in sense or antisense orientation; (c) a nucleotide sequence of interest (NSI) in antisense orientation; and (d) a conditional by inversion module (COIN, which utilizes an exon-splitting intron and an invertible gene-trap-like module) in reverse orientation. See, e.g., US 2011/0104799. The conditional allele can further comprise recombinable units that recombine upon exposure to a first recombinase to form a conditional allele that (i) lacks the actuating sequence and the DSC; and (ii) contains the NSI in sense orientation and the COIN in antisense orientation. See, e.g., US 2011/0104799.

One exemplary reverse-conditional null endogenous lysosomal storage disease gene is a reverse-conditional null endogenous Arsb gene. For example, the reverse-conditional null endogenous Arsb gene can be one in which a region comprising exon 5 of the endogenous Arsb gene is inverted and flanked by recombinase recognition sites, the recombinase recognition sites are oriented such that the region is inverted upon treatment with the recombinase, the recombinase is a Cre recombinase, and the recombinase recognition sites comprise a lox71 site and a lox66 site, and a non-human animal comprising the reverse-conditional null endogenous Arsb gene displays one or more phenotypes associated with mucopolysaccharidosis VI. In a specific example, the reverse-conditional null endogenous Arsb gene comprises the sequence set forth in SEQ ID NO: 47 or is at least about 95%, at least about 96%, at least about 97%, at least about 98%, or at least about 99% identical to SEQ ID NO: 47. In a specific example, the reverse-conditional null endogenous Arsb gene comprises the sequence set forth in SEQ ID NO: 7 or 8 or is at least about 95%, at least about 96%, at least about 97%, at least about 98%, or at least about 99% identical to SEQ ID NO: 7 or 8. In a specific example, upon treatment the reverse-conditional null endogenous Arsb gene comprises the sequence set forth in SEQ ID NO: 48 or is at least about 95%, at least about 96%, at least about 97%, at least about 98%, or at least about 99% identical to SEQ ID NO: 48. In a specific example, upon treatment the reverse-conditional null endogenous Arsb gene comprises the sequence set forth in SEQ ID NO: 9 or is at least about 95%, at least about 96%, at least about 97%, at least about 98%, or at least about 99% identical to SEQ ID NO: 9.

Another exemplary reverse-conditional null endogenous lysosomal storage disease gene is a reverse-conditional null endogenous Idua gene. For example, the reverse-conditional null endogenous Idua gene can be one in which a region comprising exon 2 to the stop codon of the endogenous Idua gene is inverted and flanked by recombinase recognition sites, the recombinase recognition sites are oriented such that the region is inverted upon treatment with the recombinase, the recombinase is a Cre recombinase, and the recombinase recognition sites comprise a lox71 site and a lox66 site, and a non-human animal comprising the reverse-conditional null endogenous Idua gene displays one or more phenotypes associated with mucopolysaccharidosis I. In a specific example, the reverse-conditional null endogenous Idua gene comprises the sequence set forth in SEQ ID NO: 49 or is at least about 95%, at least about 96%, at least about 97%, at least about 98%, or at least about 99% identical to SEQ ID NO: 49. In a specific example, the reverse-conditional null endogenous Idua gene comprises the sequence set forth in SEQ ID NO: 10 or 11 or is at least about 95%, at least about 96%, at least about 97%, at least about 98%, or at least about 99% identical to SEQ ID NO: 10 or 11. In a specific example, upon treatment the reverse-conditional null endogenous Idua gene comprises the sequence set forth in SEQ ID NO: 50 or is at least about 95%, at least about 96%, at least about 97%, at least about 98%, or at least about 99% identical to SEQ ID NO: 50. In a specific example, upon treatment the reverse-conditional null endogenous Idua gene comprises the sequence set forth in SEQ ID NO: 12 or is at least about 95%, at least about 96%, at least about 97%, at least about 98%, or at least about 99% identical to SEQ ID NO: 12.

### C. Non-Human Animal Genomes, Non-Human Animal Cells, and Non-Human Animals Comprising a Reverse-Conditional Null Endogenous Lysosomal Storage Disease Gene

Non-human animal genomes, non-human animal cells, and non-human animals comprising in their genome a reverse-conditional null endogenous lysosomal storage disease gene as described elsewhere herein are provided. The genomes, cells, or non-human animals can be male or female. The genomes, cells, or non-human animals can be heterozygous or homozygous for the reverse-conditional null endogenous lysosomal storage disease gene. A diploid organism has two alleles at each genetic locus. Each pair of alleles represents the genotype of a specific genetic locus. Genotypes are described as homozygous if there are two identical alleles at a particular locus and as heterozygous if the two alleles differ. A non-human animal comprising a reverse-conditional null endogenous lysosomal storage disease gene can comprise the reverse-conditional null endogenous lysosomal storage disease gene in its germline.

The non-human animal genomes or cells provided herein can be, for example, any non-human animal genome or cell comprising a lysosomal storage disease gene, a mucopolysaccharidosis gene, an Arsb gene, or an Idua gene or a genomic locus homologous or orthologous to a human lysosomal storage disease gene, mucopolysaccharidosis gene, Arsb gene, or Idua gene. The genomes can be from or the cells can be eukaryotic cells, which include, for example, animal cells, mammalian cells, non-human mammalian cells, and human cells. The term “animal” includes any member of the animal kingdom, including, for example, mammals, fishes, reptiles, amphibians, birds, and worms. A mammalian cell can be, for example, a non-human mammalian cell, a rodent cell, a rat cell, or a mouse cell. Other non-human mammals include, for example, non-human primates. The term “non-human” excludes humans.

The cells can also be any type of undifferentiated or differentiated state. For example, a cell can be a totipotent cell, a pluripotent cell (e.g., a human pluripotent cell or a non-human pluripotent cell such as a mouse embryonic stem (ES) cell or a rat ES cell), or a non-pluripotent cell (e.g., a non-ES cell). Totipotent cells include undifferentiated cells that can give rise to any cell type, and pluripotent cells include undifferentiated cells that possess the ability to develop into more than one differentiated cell types. Such pluripotent and/or totipotent cells can be, for example, ES cells or ES-like cells, such as an induced pluripotent stem (iPS) cells. ES cells include embryo-derived totipotent or pluripotent cells that are capable of contributing to any tissue of the developing embryo upon introduction into an embryo. ES cells can be derived from the inner cell mass of a blastocyst and are capable of differentiating into cells of any of the three vertebrate germ layers (endoderm, ectoderm, and mesoderm).

The cells provided herein can also be germ cells (e.g., sperm or oocytes). The cells can be mitotically competent cells or mitotically-inactive cells, meiotically competent cells or meiotically-inactive cells. Similarly, the cells can also be primary somatic cells or cells that are not a primary somatic cell. Somatic cells include any cell that is not a gamete, germ cell, gametocyte, or undifferentiated stem cell.

Suitable cells provided herein also include primary cells. Primary cells include cells or cultures of cells that have been isolated directly from an organism, organ, or tissue. Primary cells include cells that are neither transformed nor immortal. They include any cell obtained from an organism, organ, or tissue which was not previously passed in tissue culture or has been previously passed in tissue culture but is incapable of being indefinitely passed in tissue culture.

Other suitable cells provided herein include immortalized cells. Immortalized cells include cells from a multicellular organism that would normally not proliferate indefinitely but, due to mutation or alteration, have evaded normal cellular senescence and instead can keep undergoing division. Such mutations or alterations can occur naturally or be intentionally induced. Numerous types of immortalized cells are well known. Immortalized or primary cells include cells that are typically used for culturing or for expressing recombinant genes or proteins.

The cells provided herein also include one-cell stage embryos (i.e., fertilized oocytes or zygotes). Such one-cell stage embryos can be from any genetic background (e.g., BALB/c, C57BL/6, 129, or a combination thereof for mice), can be fresh or frozen, and can be derived from natural breeding or in vitro fertilization.

The cells provided herein can be normal, healthy cells, or can be diseased or mutant-bearing cells.

Non-human animals comprising a reverse-conditional null endogenous lysosomal storage disease gene as described herein can be made by the methods described elsewhere herein. The term “animal” includes any member of the animal kingdom, including, for example, mammals, fishes, reptiles, amphibians, birds, and worms. In a specific example, the non-human animal is a non-human mammal. Non-human mammals include, for example, non-human primates and rodents (e.g., mice and rats). The term “non-human animal” excludes humans. Preferred non-human animals include, for example, rodents, such as mice and rats.

The non-human animals can be from any genetic background. For example, suitable mice can be from a 129 strain, a C57BL/6 strain, a mix of 129 and C57BL/6, a BALB/c strain, or a Swiss Webster strain. Examples of 129 strains include 129P1, 129P2, 129P3, 129X1, 129S1 (e.g., 129S1/SV, 129S1/Sv1m), 129S2, 129S4, 129S5, 129S9/SvEvH, 129S6 (129/SvEvTac), 129S7, 129S8, 129T1, and 129T2. See, e.g., Festing et al. (1999) Mamm. Genome 10(8):836, herein incorporated by reference in its entirety for all purposes. Examples of C57BL strains include C57BL/A, C57BL/An, C57BL/GrFa, C57BL/Kal_wN, C57BL/6, C57BL/6J, C57BL/6ByJ, C57BL/6NJ, C57BL/10, C57BL/10ScSn, C57BL/10Cr, and C57BL/O1a. Suitable mice can also be from a mix of an aforementioned 129 strain and an aforementioned C57BL/6 strain (e.g., 50% 129 and 50% C57BL/6). Likewise, suitable mice can be from a mix of aforementioned 129 strains or a mix of aforementioned BL/6 strains (e.g., the 129S6 (129/SvEvTac) strain).

Similarly, rats can be from any rat strain, including, for example, an ACI rat strain, a Dark Agouti (DA) rat strain, a Wistar rat strain, a LEA rat strain, a Sprague Dawley (SD) rat strain, or a Fischer rat strain such as Fisher F344 or Fisher F6. Rats can also be obtained from a strain derived from a mix of two or more strains recited above. For example, a suitable rat can be from a DA strain or an ACI strain. The ACI rat strain is characterized as having black agouti, with white belly and feet and an RT1^{av1} haplotype. Such strains are available from a variety of sources including Harlan Laboratories. The Dark Agouti (DA) rat strain is characterized as having an agouti coat and an RT1^{av1} haplotype. Such rats are available from a variety of sources including Charles River and Harlan Laboratories. Some suitable rats can be from an inbred rat strain. See, e.g., US 2014/0235933, herein incorporated by reference in its entirety for all purposes.

In one specific example, a non-human animal (e.g., mouse) comprising in its genome a reverse-conditional null endogenous lysosomal storage disease gene as described elsewhere herein comprises a reverse-conditional null endogenous Arsb gene. The non-human animal can display one or more phenotypes associated with mucopolysaccharidosis VI, such as one or more skeletal phenotypes associated with mucopolysaccharidosis VI. For example, the non-human animal can display one or more or all of the following relative to a wild type non-human animal: (a) increased accumulation of glycosaminoglycans in the liver; (b) increased accumulation of glycosaminoglycans in the heart; (c) increased accumulation of glycosaminoglycans in the kidney; (d) decreased tibia length; (e) decreased spinal column length; (f) increased tibial growth plate width; and (g) decreased cranial length:width ratio. In one example, the non-human animal displays increased accumulation of glycosaminoglycans in the liver. In another example, the non-human animal displays increased accumulation of glycosaminoglycans in the heart. In another example, the non-human animal displays increased accumulation of glycosaminoglycans in the kidney. In another example, the non-human animal displays decreased tibia length. In another example, the non-human animal displays decreased spinal column length. In another example, the non-human animal displays increased tibial growth plate width. In another example, the non-human animal displays decreased cranial length:width ratio. In another example, the non-human animal displays: (a) increased accumulation of glycosaminoglycans in the liver; (b) increased accumulation of glycosaminoglycans in the heart; (c) increased accumulation of glycosaminoglycans in the kidney; (d) decreased tibia length; (e) decreased spinal column length; (f) increased tibial growth plate width; and (g) decreased cranial length:width ratio. In a specific example, the non-human animal displays the decreased tibia length, wherein the decreased tibia length is rescuable within one month after treatment with the recombinase at post-natal day 7 (or earlier) but is not rescuable within one month after treatment with the recombinase at post-natal day 21 (or later) or at 8-10 weeks (or later) (e.g., 8 weeks (or later)) after birth. In another specific example, the non-human animal displays the decreased spinal column length, wherein the decreased spinal column length is rescuable within one month after treatment with the recombinase at post-natal day 7 (or earlier) but is not rescuable within one month after treatment with the recombinase at post-natal day 21 (or later) or at 8-10 weeks (or later) (e.g., 8 weeks (or later)) after birth. In another specific example, the non-human animal displays the increased tibial growth plate width, wherein the increased tibial growth plate width is rescuable within one month after treatment with the recombinase at post-natal day 7 (or earlier) but is not rescuable within one month after treatment with the recombinase at post-natal day 21 (or later) or at 8-10 weeks (or later) (e.g., 8 weeks (or later)) after birth. In another specific example, the non-human animal displays the decreased cranial length:width ratio, wherein the decreased cranial length:width ratio is rescuable within one month after treatment with the recombinase at post-natal day 7 (or earlier) but is not rescuable within one month after treatment with the recombinase at post-natal day 21 (or later) or at 8-10 weeks (or later) (e.g., 8 weeks (or later)) after birth. In a specific example, the non-human animal displays the decreased tibia length, wherein the decreased tibia length is rescuable within three months after treatment with the recombinase at post-natal day 7 (or earlier) but is not rescuable within three months after treatment with the recombinase at post-natal day 21 (or later) or at 8-10 weeks (or later) (e.g., 8 weeks (or later)) after birth. In another specific example, the non-human animal displays the decreased spinal column length, wherein the decreased spinal column length is rescuable within three months after treatment with the recombinase at post-natal day 7 (or earlier) but is not rescuable within three months after treatment with the recombinase at post-natal day 21 (or later) or at 8-10 weeks (or later) (e.g., 8 weeks (or later)) after birth. In another specific example, the non-human animal displays the increased tibial growth plate width, wherein the increased tibial growth plate width is rescuable within three months after treatment with the recombinase at post-natal day 7 (or earlier) but is not rescuable within three months after treatment with the recombinase at post-natal day 21 (or later) or at 8-10 weeks (or later) (e.g., 8 weeks (or later)) after birth. In another specific example, the non-human animal displays the decreased cranial length:width ratio, wherein the decreased cranial length:width ratio is rescuable within three months after treatment with the recombinase at post-natal day 7 (or earlier) but is not rescuable within three months after treatment with the recombinase at post-natal day 21 (or later) or at 8-10 weeks (or later) (e.g., 8 weeks (or later)) after birth.

In another specific example, a non-human animal (e.g., mouse) comprising in its genome a reverse-conditional null endogenous lysosomal storage disease gene as described elsewhere herein comprises a reverse-conditional null endogenous Idua gene. The non-human animal can display one or more phenotypes associated with mucopolysaccharidosis I. The non-human animal can display one or more skeletal phenotypes associated with mucopolysaccharidosis I. For example, the non-human animal can display one or more or all of the following relative to a wild type non-human animal: (a) increased accumulation of glycosaminoglycans in the liver; (b) increased accumulation of glycosaminoglycans in the heart; (c) increased accumulation of glycosaminoglycans in the kidney; (d) decreased tibia length; (e) decreased spinal column length; (f) decreased ribcage width; and (g) decreased cranial length:width ratio. In one example, the non-human animal displays increased accumulation of glycosaminoglycans in the liver. In another example, the non-human animal displays increased accumulation of glycosaminoglycans in the heart. In another example, the non-human animal displays increased accumulation of glycosaminoglycans in the kidney. In another example, the non-human animal displays decreased tibia length. In another example, the non-human animal displays decreased spinal column length. In another example, the non-human animal displays decreased ribcage width. In another example, the non-human animal displays decreased cranial length:width ratio. In another example, the non-human animal displays: (a) increased accumulation of glycosaminoglycans in the liver; (b) increased accumulation of glycosaminoglycans in the heart; (c) increased accumulation of glycosaminoglycans in the kidney; (d) decreased tibia length; (e) decreased spinal column length; (f) decreased ribcage width; and (g) decreased cranial length:width ratio. The non-human animal can display one or more neurological phenotypes associated with mucopolysaccharidosis I. For example, the non-human animal can display one or more or all of the following relative to a wild type non-human animal: (a) decreased exploratory behavior (e.g., decreased number of rearings in an open field test); (b) decreased anxiety-related behavior (e.g., in marble-burying tasks or in an elevated plus maze); (c) impaired long-term memory retention (e.g., in a fear conditioning and memory test); and (d) increased escape latency (e.g., in a Barnes maze). In one example, the non-human animal displays decreased exploratory behavior (e.g., decreased number of rearings in an open field test). In another example, the non-human animal displays decreased anxiety-related behavior (e.g., in marble-burying tasks or in an elevated plus maze). In another example, the non-human animal displays impaired long-term memory retention (e.g., in a fear conditioning and memory test). In another example, the non-human animal displays increased escape latency (e.g., in a Barnes maze). In another example, the non-human animal displays: (a) decreased exploratory behavior (e.g., decreased number of rearings in an open field test); (b) decreased anxiety-related behavior (e.g., in marble-burying tasks or in an elevated plus maze); (c) impaired long-term memory retention (e.g., in a fear conditioning and memory test); and (d) increased escape latency (e.g., in a Barnes maze). The non-human animal can display one or more skeletal phenotypes associated with mucopolysaccharidosis I and one or more neurological phenotypes associated with mucopolysaccharidosis I.

### III. Methods of Making Non-Human Animals Comprising a Reverse-Conditional Null Endogenous Lysosomal Storage Disease Gene

Various methods are provided for making a non-human animal genome, non-human animal cell, or non-human animal comprising a reverse-conditional null endogenous lysosomal storage disease gene (e.g., a reverse-conditional null endogenous mucopolysaccharidosis gene, a reverse-conditional null endogenous Arsb gene, or a reverse-conditional null endogenous Idua gene) as disclosed elsewhere herein. Likewise, various methods are provided for making a reverse-conditional null endogenous lysosomal storage disease gene (e.g., a reverse-conditional null endogenous mucopolysaccharidosis gene, a reverse-conditional null endogenous Arsb gene, or a reverse-conditional null endogenous Idua gene) or for making a non-human animal genome or non-human animal cell comprising a reverse-conditional null endogenous lysosomal storage disease gene (e.g., a reverse-conditional null endogenous mucopolysaccharidosis gene, a reverse-conditional null endogenous Arsb gene, or a reverse-conditional null endogenous Idua gene) as disclosed elsewhere herein. Any convenient method or protocol for producing a genetically modified organism is suitable for producing such a genetically modified non-human animal. See, e.g., Poueymirou et al. (2007) Nat. Biotechnol. 25(1):91-99; U.S. Pat. Nos. 7,294,754; 7,576,259; 7,659,442; 8,816,150; 9,414,575; 9,730,434; and 10,039,269, each of which is herein incorporated by reference in its entirety for all purposes (describing mouse ES cells and the VELOCIMOUSE® method for making a genetically modified mouse). See also US 2014/0235933 A1, US 2014/0310828 A1, each of which is herein incorporated by reference in its entirety for all purposes (describing rat ES cells and methods for making a genetically modified rat). See also Cho et al. (2009) Curr. Protoc. Cell. Biol. 42:19.11.1-19.11.22 (doi: 10.1002/0471143030.cb1911s42) and Gama Sosa et al. (2010) Brain Struct. Funct. 214(2-3):91-109, each of which is herein incorporated by reference in its entirety for all purposes. Such genetically modified non-human animals can be generated, for example, through gene knock-in at a targeted lysosomal storage disease, mucopolysaccharidosis, Arsb, or Idua locus.

For example, the method of producing a non-human animal comprising the reverse-conditional null endogenous lysosomal storage disease gene can comprise: (1) providing a pluripotent cell (e.g., an embryonic stem (ES) cell such as a mouse ES cell or a rat ES cell) comprising the reverse-conditional null endogenous lysosomal storage disease gene; (2) introducing the genetically modified pluripotent cell into a non-human animal host embryo; and (3) gestating the host embryo in a surrogate non-human animal mother.

As another example, the method of producing a non-human animal comprising a reverse-conditional null endogenous lysosomal storage disease gene can comprise: (1) modifying the genome of a non-human animal pluripotent cell (e.g., an embryonic stem (ES) cell such as a mouse ES cell or a rat ES cell) to comprise the reverse-conditional null endogenous lysosomal storage disease gene; (2) identifying or selecting the genetically modified non-human animal pluripotent cell comprising the reverse-conditional null endogenous lysosomal storage disease gene; (3) introducing the genetically modified non-human animal pluripotent cell into a non-human animal host embryo; and (4) gestating the non-human animal host embryo in a non-human animal surrogate mother. The donor cell can be introduced into a host embryo at any stage, such as the blastocyst stage or the pre-morula stage (i.e., the 4 cell stage or the 8 cell stage). Optionally, the host embryo comprising modified pluripotent cell (e.g., a non-human ES cell) can be incubated until the blastocyst stage before being implanted into and gestated in the surrogate mother to produce an F0 non-human animal. The surrogate mother can then produce an F0 generation non-human animal comprising the reverse-conditional null endogenous lysosomal storage disease gene (and capable of transmitting the genetic modification through the germline).

Alternatively, the method of producing the non-human animals described elsewhere herein can comprise: (1) modifying the genome of a non-human animal one-cell stage embryo to comprise the reverse-conditional null endogenous lysosomal storage disease gene (e.g., using the methods described above for modifying pluripotent cells); (2) selecting the genetically modified non-human animal embryo; and (3) gestating the genetically modified non-human animal embryo in a surrogate non-human animal mother. Progeny that are capable of transmitting the genetic modification though the germline are generated.

Nuclear transfer techniques can also be used to generate the non-human mammalian animals. Briefly, methods for nuclear transfer can include the steps of: (1) enucleating an oocyte or providing an enucleated oocyte; (2) isolating or providing a donor cell or nucleus to be combined with the enucleated oocyte; (3) inserting the cell or nucleus into the enucleated oocyte to form a reconstituted cell; (4) implanting the reconstituted cell into the womb of an animal to form an embryo; and (5) allowing the embryo to develop. In such methods, oocytes are generally retrieved from deceased animals, although they may be isolated also from either oviducts and/or ovaries of live animals. Oocytes can be matured in a variety of well-known media prior to enucleation. Enucleation of the oocyte can be performed in a number of well-known manners. Insertion of the donor cell or nucleus into the enucleated oocyte to form a reconstituted cell can be by microinjection of a donor cell under the zona pellucida prior to fusion. Fusion may be induced by application of a DC electrical pulse across the contact/fusion plane (electrofusion), by exposure of the cells to fusion-promoting chemicals, such as polyethylene glycol, or by way of an inactivated virus, such as the Sendai virus. A reconstituted cell can be activated by electrical and/or non-electrical means before, during, and/or after fusion of the nuclear donor and recipient oocyte. Activation methods include electric pulses, chemically induced shock, penetration by sperm, increasing levels of divalent cations in the oocyte, and reducing phosphorylation of cellular proteins (as by way of kinase inhibitors) in the oocyte. The activated reconstituted cells, or embryos, can be cultured in well-known media and then transferred to the womb of an animal. See, e.g., US 2008/0092249, WO 1999/005266, US 2004/0177390, WO 2008/017234, and U.S. Pat. No. 7,612,250, each of which is herein incorporated by reference in its entirety for all purposes.

The modified cell or one-cell stage embryo can be generated, for example, through recombination by (a) introducing into the cell one or more exogenous donor nucleic acids (e.g., targeting vectors) comprising an insert nucleic acid flanked, for example, by 5′ and 3′ homology arms corresponding to 5′ and 3′ target sites (e.g., target sites flanking the endogenous sequences intended for deletion and replacement with the insert nucleic acid), wherein the insert nucleic acid comprises a portion of the reverse-conditional null endogenous lysosomal storage disease gene (e.g., an inverted portion of the endogenous lysosomal storage disease gene flanked by recombinase recognition sites) to generate the reverse-conditional null endogenous lysosomal storage disease gene; and (b) identifying at least one cell comprising in its genome the insert nucleic acid integrated at the endogenous lysosomal storage disease gene locus (i.e., identifying at least one cell comprising the reverse-conditional null endogenous lysosomal storage disease gene). Likewise, a modified non-human animal genome or humanized non-human animal reverse-conditional null endogenous lysosomal storage disease gene can be generated, for example, through recombination by (a) contacting the genome or gene with one or more exogenous donor nucleic acids (e.g., targeting vectors) comprising 5′ and 3′ homology arms corresponding to 5′ and 3′ target sites (e.g., target sites flanking the endogenous sequences intended for deletion and replacement with an insert nucleic acid (e.g., comprising a portion of the reverse-conditional null endogenous lysosomal storage disease gene (e.g., an inverted portion of the endogenous lysosomal storage disease gene flanked by recombinase recognition sites) to generate the reverse-conditional null endogenous lysosomal storage disease gene) flanked by the 5′ and 3′ homology arms), wherein the exogenous donor nucleic acids are designed for generating the reverse-conditional null endogenous lysosomal storage disease gene.

Alternatively, the modified pluripotent cell or one-cell stage embryo can be generated by (a) introducing into the cell: (i) a nuclease agent, wherein the nuclease agent induces a nick or double-strand break at a target site within the endogenous lysosomal storage disease gene locus; and (ii) one or more exogenous donor nucleic acids (e.g., targeting vectors) comprising an insert nucleic acid flanked by, for example, 5′ and 3′ homology arms corresponding to 5′ and 3′ target sites (e.g., target sites flanking the endogenous sequences intended for deletion and replacement with the insert nucleic acid), wherein the insert nucleic acid comprises a portion of the reverse-conditional null endogenous lysosomal storage disease gene (e.g., an inverted portion of the endogenous lysosomal storage disease gene flanked by recombinase recognition sites) to generate the reverse-conditional null endogenous lysosomal storage disease gene; and (c) identifying at least one cell comprising in its genome the insert nucleic acid integrated at the endogenous lysosomal storage disease gene locus (i.e., identifying at least one cell comprising the reverse-conditional null endogenous lysosomal storage disease gene). Likewise, a modified non-human animal genome or humanized non-human animal reverse-conditional null endogenous lysosomal storage disease gene can be generated by contacting the genome or gene with: (i) a nuclease agent, wherein the nuclease agent induces a nick or double-strand break at a target site within the endogenous lysosomal storage disease locus or gene; and (ii) one or more exogenous donor nucleic acids (e.g., targeting vectors) comprising an insert nucleic acid (e.g., comprising a portion of the reverse-conditional null endogenous lysosomal storage disease gene (e.g., an inverted portion of the endogenous lysosomal storage disease gene flanked by recombinase recognition sites) to generate reverse-conditional null endogenous lysosomal storage disease gene) flanked by, for example, 5′ and 3′ homology arms corresponding to 5′ and 3′ target sites (e.g., target sites flanking the endogenous sequences intended for deletion and replacement with the insert nucleic acid), wherein the exogenous donor nucleic acids are designed for humanization of the endogenous lysosomal storage disease gene locus. Any nuclease agent that induces a nick or double-strand break into a desired recognition site can be used. Examples of suitable nucleases include a Transcription Activator-Like Effector Nuclease (TALEN), a zinc-finger nuclease (ZFN), a meganuclease, and Clustered Regularly Interspersed Short Palindromic Repeats (CRISPR)/CRISPR-associated (Cas) systems (e.g., CRISPR/Cas9 systems) or components of such systems (e.g., CRISPR/Cas9). See, e.g., US 2013/0309670 and US 2015/0159175, each of which is herein incorporated by reference in its entirety for all purposes. In one example, the nuclease comprises a Cas9 protein and a guide RNA. In another example, the nuclease comprises a Cas9 protein and two or more, three or more, or four or more guide RNAs.

The step of modifying the genome can, for example, utilize exogenous repair templates (e.g., targeting vectors) to modify an endogenous lysosomal storage disease gene locus to comprise the reverse-conditional null endogenous lysosomal storage disease gene locus disclosed herein. As one example, the targeting vector can be for generating the reverse-conditional null endogenous lysosomal storage disease gene, wherein the targeting vector comprises a nucleic acid insert comprising a portion of the reverse-conditional null endogenous lysosomal storage disease gene (e.g., an inverted portion of the endogenous lysosomal storage disease gene flanked by recombinase recognition sites) flanked by a 5′ homology arm targeting a 5′ target sequence at the endogenous lysosomal storage disease gene locus and a 3′ homology arm targeting a 3′ target sequence at the endogenous lysosomal storage disease gene locus. Integration of a nucleic acid insert in the endogenous lysosomal storage disease gene locus can result in addition of a nucleic acid sequence of interest in the endogenous lysosomal storage disease gene locus, deletion of a nucleic acid sequence of interest in the endogenous lysosomal storage disease gene locus, or replacement of a nucleic acid sequence of interest in the endogenous lysosomal storage disease gene locus (i.e., deleting a segment of the endogenous lysosomal storage disease gene locus and replacing with a portion of the reverse-conditional null endogenous lysosomal storage disease gene (e.g., an inverted portion of the endogenous lysosomal storage disease gene flanked by recombinase recognition sites)).

The exogenous repair templates can be for non-homologous-end-joining-mediated insertion or homologous recombination. Exogenous repair templates can comprise deoxyribonucleic acid (DNA) or ribonucleic acid (RNA), they can be single-stranded or double-stranded, and they can be in linear or circular form. For example, a repair template can be a single-stranded oligodeoxynucleotide (ssODN). Exogenous repair templates can also comprise a heterologous sequence that is not present at an untargeted endogenous lysosomal storage disease gene locus. For example, an exogenous repair template can comprise a selection cassette, such as a selection cassette flanked by recombinase recognition sites.

In cells other than one-cell stage embryos, the exogenous repair template can be a “large targeting vector” or “LTVEC,” which includes targeting vectors that comprise homology arms that correspond to and are derived from nucleic acid sequences larger than those typically used by other approaches intended to perform homologous recombination in cells. See, e.g., US 2004/0018626; WO 2013/163394; U.S. Pat. Nos. 9,834,786; 10,301,646; WO 2015/088643; U.S. Pat. Nos. 9,228,208; 9,546,384; 10,208,317; and US 2019-0112619, each of which is herein incorporated by reference in its entirety for all purposes. LTVECs also include targeting vectors comprising nucleic acid inserts having nucleic acid sequences larger than those typically used by other approaches intended to perform homologous recombination in cells. For example, LTVECs make possible the modification of large loci that cannot be accommodated by traditional plasmid-based targeting vectors because of their size limitations. For example, the targeted locus can be (i.e., the 5′ and 3′ homology arms can correspond to) a locus of the cell that is not targetable using a conventional method or that can be targeted only incorrectly or only with significantly low efficiency in the absence of a nick or double-strand break induced by a nuclease agent (e.g., a Cas protein). LTVECs can be of any length and are typically at least 10 kb in length. The sum total of the 5′ homology arm and the 3′ homology arm in an LTVEC is typically at least 10 kb. Generation and use of large targeting vectors (LTVECs) derived from bacterial artificial chromosome (BAC) DNA through bacterial homologous recombination (BHR) reactions using VELOCIGENE® genetic engineering technology is described, e.g., in U.S. Pat. No. 6,586,251 and Valenzuela et al. (2003) Nat. Biotechnol. 21(6):652-659, each of which is herein incorporated by reference in its entirety for all purposes. Generation of LTVECs through in vitro assembly methods is described, e.g., in US 2015/0376628 and WO 2015/200334, each of which is herein incorporated by reference in its entirety for all purposes.

The methods can further comprise identifying a cell or animal having a modified target genomic locus. Various methods can be used to identify cells and animals having a targeted genetic modification. The screening step can comprise, for example, a quantitative assay for assessing modification-of-allele (MOA) of a parental chromosome. See, e.g., US 2004/0018626; US 2014/0178879; US 2016/0145646; WO 2016/081923; and Frendewey et al. (2010) Methods Enzymol. 476:295-307, each of which is herein incorporated by reference in its entirety for all purposes. For example, the quantitative assay can be carried out via a quantitative PCR, such as a real-time PCR (qPCR). The real-time PCR can utilize a first primer set that recognizes the target locus and a second primer set that recognizes a non-targeted reference locus. The primer set can comprise a fluorescent probe that recognizes the amplified sequence. Other examples of suitable quantitative assays include fluorescence-mediated in situ hybridization (FISH), comparative genomic hybridization, isothermic DNA amplification, quantitative hybridization to an immobilized probe(s), INVADER® Probes, TAQMAN® Molecular Beacon probes, or ECLIPSE™ probe technology (see, e.g., US 2005/0144655, incorporated herein by reference in its entirety for all purposes).

The various methods provided herein allow for the generation of a genetically modified non-human F0 animal wherein the cells of the genetically modified F0 animal comprise the reverse-conditional null endogenous lysosomal storage disease gene. It is recognized that depending on the method used to generate the F0 animal, the number of cells within the F0 animal that have the reverse-conditional null endogenous lysosomal storage disease gene will vary. With mice, for example, the introduction of the donor ES cells into a pre-morula stage embryo from the mouse (e.g., an 8-cell stage mouse embryo) via, for example, the VELOCIMOUSE® method allows for a greater percentage of the cell population of the F0 mouse to comprise cells having the targeted genetic modification. For example, at least 50%, 60%, 65%, 70%, 75%, 85%, 86%, 87%, 87%, 88%, 89%, 90%, 91%, 92%, 93%, 94%, 95%, 96%, 97%, 98%, 99%, or 100% of the cellular contribution of the non-human F0 animal can comprise a cell population having the targeted modification. The cells of the genetically modified F0 animal can be heterozygous for the reverse-conditional null endogenous lysosomal storage disease gene or can be homozygous for the reverse-conditional null endogenous lysosomal storage disease gene.

### IV. Methods of Using Non-Human Animals Comprising a Reverse-Conditional Null Endogenous Lysosomal Storage Disease Gene

Various methods are provided for using the non-human animals comprising a reverse-conditional null endogenous lysosomal storage disease gene as described elsewhere herein. Such methods include, e.g., assessing reversibility of a phenotype of the lysosomal storage disease or determining an optimal timeframe for treatment of a phenotype of the lysosomal storage disease.

### A. Methods of Assessing Reversibility of a Lysosomal Storage Disease Phenotype

Various methods are provided for assessing reversibility of a phenotype of the lysosomal storage disease using non-human animals comprising a reverse-conditional null endogenous lysosomal storage disease gene as described elsewhere herein. Such methods can comprise: (a) treating the non-human animal with the recombinase at a selected time point after birth or treating the non-human animal with a candidate therapeutic agent at the selected time point after birth; and (b) assessing the phenotype of the lysosomal storage disease after a defined time period post-treatment (e.g., relative to a control non-human animal).

In some methods, step (a) comprises treating the non-human animal with the candidate therapeutic agent at the selected time point after birth. In some methods, step (a) comprises treating the non-human animal with the candidate therapeutic agent at the selected time point after birth but does not comprise treating the non-human animal with the recombinase. In some methods, step (a) comprises treating the non-human animal with the recombinase at the selected time point after birth. In some methods, step (a) comprises treating the non-human animal with the recombinase at the selected time point after birth but does not comprise treating the non-human animal with the candidate therapeutic agent. In some methods, step (a) comprises treating the non-human animal with the recombinase and the candidate therapeutic agent at the selected time point after birth or at separate time points after birth. For example, step (a) can comprise treating the non-human animal with the recombinase at a first time point and treating the non-human animal with the candidate therapeutic agent at a second time point after birth, where the first time point can be before or after the second time point.

The candidate therapeutic agent can be any candidate therapeutic agent, and the non-human animal can be treated with the candidate therapeutic agent by any suitable means. A candidate therapeutic agent a known therapeutic agent for a lysosomal storage disease (e.g., MPS VI or MPS I) or can be a putative therapeutic agent for a lysosomal storage disease,

For example, a candidate therapeutic agent can be an antigen-binding protein. The term “antigen-binding protein” includes any protein that binds to an antigen. Examples of antigen-binding proteins include an antibody, an antigen-binding fragment of an antibody, a multispecific antibody (e.g., a bi-specific antibody), an scFV, a bis-scFV, a diabody, a triabody, a tetrabody, a V-NAR, a VHH, a VL, a F(ab), a F(ab)₂, a DVD (dual variable domain antigen-binding protein), an SVD (single variable domain antigen-binding protein), a bispecific T-cell engager (BiTE), or a Davisbody (U.S. Pat. No. 8,586,713, herein incorporated by reference herein in its entirety for all purposes).

In another example, a candidate therapeutic agent can be a small molecule. Other candidate therapeutic agents can include genome editing reagents such as a nuclease agent (e.g., a Clustered Regularly Interspersed Short Palindromic Repeats (CRISPR)/CRISPR-associated (Cas) (CRISPR/Cas) nuclease, a zinc finger nuclease (ZFN), or a Transcription Activator-Like Effector Nuclease (TALEN)).

Other candidate therapeutic agents can include RNAi agents. An “RNAi agent” is a composition that comprises a small double-stranded RNA or RNA-like (e.g., chemically modified RNA) oligonucleotide molecule capable of facilitating degradation or inhibition of translation of a target RNA, such as messenger RNA (mRNA), in a sequence-specific manner. The oligonucleotide in the RNAi agent is a polymer of linked nucleosides, each of which can be independently modified or unmodified. RNAi agents operate through the RNA interference mechanism (i.e., inducing RNA interference through interaction with the RNA interference pathway machinery (RNA-induced silencing complex or RISC) of mammalian cells). While it is believed that RNAi agents, as that term is used herein, operate primarily through the RNA interference mechanism, the disclosed RNAi agents are not bound by or limited to any particular pathway or mechanism of action. RNAi agents disclosed herein comprise a sense strand and an antisense strand, and include, but are not limited to, short interfering RNAs (siRNAs), double-stranded RNAs (dsRNA), micro RNAs (miRNAs), short hairpin RNAs (shRNA), and dicer substrates. The antisense strand of the RNAi agents described herein is at least partially complementary to a sequence (i.e., a succession or order of nucleobases or nucleotides, described with a succession of letters using standard nomenclature) in the target RNA.

Other candidate therapeutic agents can include antisense oligonucleotides (ASOs). Single-stranded ASOs and RNA interference (RNAi) share a fundamental principle in that an oligonucleotide binds a target RNA through Watson-Crick base pairing. Without wishing to be bound by theory, during RNAi, a small RNA duplex (RNAi agent) associates with the RNA-induced silencing complex (RISC), one strand (the passenger strand) is lost, and the remaining strand (the guide strand) cooperates with RISC to bind complementary RNA. Argonaute 2 (Ago2), the catalytic component of the RISC, then cleaves the target RNA. The guide strand is always associated with either the complementary sense strand or a protein (RISC). In contrast, an ASO must survive and function as a single strand. ASOs bind to the target RNA and block ribosomes or other factors, such as splicing factors, from binding the RNA or recruit proteins such as nucleases. Different modifications and target regions are chosen for ASOs based on the desired mechanism of action. A gapmer is an ASO oligonucleotide containing 2-5 chemically modified nucleotides (e.g. LNA or 2′-MOE) on each terminus flanking a central 8-10 base gap of DNA. After binding the target RNA, the DNA-RNA hybrid acts substrate for RNase H.

Other candidate therapeutic agents include proteins or nucleic acids encoding proteins. In one example, the candidate therapeutic agent is a protein encoded by the endogenous lysosomal storage disease gene or a nucleic acid encoding the protein. In another example, the candidate therapeutic agent is not a protein encoded by the endogenous lysosomal storage disease gene or a nucleic acid encoding the protein.

The non-human animal can be treated with the recombinase by any possible means. For example, non-human animal can further comprise a genomically integrated recombinase expression cassette encoding the recombinase, wherein the recombinase or the expression cassette is inducible, and step (a) can comprise inducing the recombinase or inducing expression of the recombinase from the inducible genomically integrated recombinase expression cassette. For example, the recombinase coding sequence can be operably linked to an inducible promoter. Expression of the recombinase can also be inducible upon treatment with tamoxifen, and treating the non-human animal with the recombinase in step (a) is treating the non-human animal with tamoxifen to induce the recombinase. The non-human animal can be treated with any suitable number of doses of tamoxifen. The non-human animal can be treated once with tamoxifen or multiple times with tamoxifen. For example, the non-human animal can be treated with about 1 to about 10, about 1 to about 9, about 1 to about 8, about 1 to about 7, about 1 to about 6, about 1 to about 5, about 1 to about 4, about 1 to about 3, about 1 to about 2, about 2 to about 10, about 3 to about 10, about 4 to about 10, about 5 to about 10, about 6 to about 10, about 7 to about 10, about 8 to about 10, about 9 to about 10, about 2 to about 8, about 3 to about 7, about 4 to about 6, or about 5 tamoxifen doses. If multiple doses of tamoxifen are given, they can be separated by any suitable amount of time. For example, the tamoxifen doses can be about every 12 hours, daily, every 2 days, or every 3 days. In a specific example, 5 daily tamoxifen doses are given. The non-human animal can be treated with any suitable concentration of tamoxifen. For example, the tamoxifen dose(s) can be about 0.25 to about 5, about 0.25 to about 4.5, about 0.25 to about 4, about 0.25 to about 3.5, about 0.25 to about 3, about 0.25 to about 2.5, about 0.25 to about 2, about 0.25 to about 1.5, about 0.25 to about 1, about 0.5 to about 5, about 0.75 to about 5, about 1 to about 5, about 1.5 to about 5, about 2 to about 5, about 2.5 to about 5, about 3 to about 5, about 3.5 to about 5, about 4 to about 5, about 4.5 to about 5, about 0.5 to about 4, about 0.75 to about 3, about 1 to about 3, about 1 to about 4, about 1 to about 5, about 1 to about 2, about 1, or about 2 mg per 25 g body weight. In one specific example, the dose of tamoxifen is about 1 mg per 25 g body weight (e.g., at P7). In another specific example, the dose of tamoxifen is about 2 mg per 25 g body weight (e.g., at P21 or at about 8 weeks after birth). A specific example is a tamoxifen-inducible Cre recombinase, which can utilize a tamoxifen inducible Cre-estrogen receptor (ER) fusion protein. Cre is fused with the ligand-binding domain of a mutated ligand-binding domain of ER, which does not bind to estrogen but binds to tamoxifen with high affinity. Cre-ER proteins are sequestered in the cytoplasm via association with the HSP90 chaperone. Upon addition of tamoxifen, tamoxifen-bound Cre-ER dissociates from HSP90, translocates into the nucleus, and carries out site-specific recombination between flanking lox sites. In a specific example, the Cre recombinase is Cre-ER^{T2}, in which additional mutations have been engineered into the ER ligand-binding domain to bind tamoxifen with a higher affinity.

In another example, the non-human animal can further comprise a genomically integrated recombinase expression cassette encoding the recombinase, wherein the recombinase coding sequence is operably linked to a tissue-specific promoter, wherein the recombinase is expressed from the promoter in a tissue-specific manner.

In another example, the non-human animal can further comprise a genomically integrated recombinase expression cassette encoding the recombinase, wherein the recombinase coding sequence is operably linked to a developmentally regulated promoter, wherein the recombinase is expressed from the promoter at a particular developmental stage.

In other examples, the non-human animal can be administered the recombinase in protein form or in the form of a nucleic acid encoding the recombinase. The recombinase or other reagents (e.g., tamoxifen or candidate therapeutic agents) can be administered by any delivery method (e.g., AAV, LNP, HDD, or injection) and by any route of administration. Administering or introducing includes presenting to the cell or non-human animal the reagents (e.g., nucleic acid or protein) in such a manner that it gains access to the interior of the cell or to the interior of cells within the non-human animal.

Recombinases can be provided in any form. For example, a recombinase can be provided in the form of a protein. Alternatively, a recombinase can be provided in the form of a nucleic acid encoding the recombinase, such as an RNA (e.g., messenger RNA (mRNA)) or DNA. Optionally, the nucleic acid encoding the recombinase can be codon optimized for efficient translation into protein in a particular cell or organism. For example, the nucleic acid encoding the recombinase can be modified to substitute codons having a higher frequency of usage in a mammalian cell, a rodent cell, a mouse cell, a rat cell, or any other host cell of interest, as compared to the naturally occurring polynucleotide sequence. When a nucleic acid encoding the recombinase is introduced into a non-human animal, the recombinase can be transiently, conditionally, or constitutively expressed in a cell in the non-human animal.

Nucleic acids encoding recombinases can be operably linked to a promoter in an expression construct. Expression constructs include any nucleic acid constructs capable of directing expression of a gene or other nucleic acid sequence of interest (e.g., a recombinase) and which can transfer such a nucleic acid sequence of interest to a target cell. Suitable promoters that can be used can be, for example, conditional promoters, inducible promoters, constitutive promoters, or tissue-specific promoters.

Various methods and compositions are provided herein to allow for introduction of reagents (e.g., a nucleic acid or protein) into a cell or non-human animal. Methods for introducing reagents into various cell types are known and include, for example, stable transfection methods, transient transfection methods, and virus-mediated methods.

Transfection protocols as well as protocols for introducing reagents into cells may vary. Non-limiting transfection methods include chemical-based transfection methods using liposomes; nanoparticles; calcium phosphate (Graham et al. (1973) Virology 52 (2): 456-67, Bacchetti et al. (1977)Proc. Natl. Acad. Sci. U.S.A. 74 (4): 1590-4, and Kriegler, M (1991). Transfer and Expression: A Laboratory Manual. New York: W. H. Freeman and Company. pp. 96-97); dendrimers; or cationic polymers such as DEAE-dextran or polyethylenimine. Non-chemical methods include electroporation, sonoporation, and optical transfection. Particle-based transfection includes the use of a gene gun, or magnet-assisted transfection (Bertram (2006) Current Pharmaceutical Biotechnology 7, 277-28). Viral methods can also be used for transfection.

Introduction of reagents (e.g., nucleic acids or proteins) into a cell can also be mediated by electroporation, by intracytoplasmic injection, by viral infection, by adenovirus, by adeno-associated virus, by lentivirus, by retrovirus, by transfection, by lipid-mediated transfection, or by nucleofection. Nucleofection is an improved electroporation technology that enables nucleic acid substrates to be delivered not only to the cytoplasm but also through the nuclear membrane and into the nucleus. In addition, use of nucleofection in the methods disclosed herein typically requires much fewer cells than regular electroporation (e.g., only about 2 million compared with 7 million by regular electroporation). In one example, nucleofection is performed using the LONZA® NUCLEOFECTOR™ system.

Introduction of reagents (e.g., nucleic acids or proteins) into a cell (e.g., a zygote) can also be accomplished by microinjection. In zygotes (i.e., one-cell stage embryos), microinjection can be into the maternal and/or paternal pronucleus or into the cytoplasm. If the microinjection is into only one pronucleus, the paternal pronucleus is preferable due to its larger size. Microinjection of an mRNA is preferably into the cytoplasm (e.g., to deliver mRNA directly to the translation machinery), while microinjection of protein or a polynucleotide encoding a protein is preferable into the nucleus/pronucleus. Alternatively, microinjection can be carried out by injection into both the nucleus/pronucleus and the cytoplasm: a needle can first be introduced into the nucleus/pronucleus and a first amount can be injected, and while removing the needle from the one-cell stage embryo a second amount can be injected into the cytoplasm. Methods for carrying out microinjection are well known. See, e.g., Nagy et al. (Nagy A, Gertsenstein M, Vintersten K, Behringer R., 2003, Manipulating the Mouse Embryo. Cold Spring Harbor, New York: Cold Spring Harbor Laboratory Press); see also Meyer et al. (2010) Proc. Natl. Acad. Sci. U.S.A. 107:15022-15026 and Meyer et al. (2012) Proc. Natl. Acad. Sci. U.S.A. 109:9354-9359.

Other methods for introducing reagents (e.g., nucleic acid or proteins) into a cell or non-human animal can include, for example, vector delivery, particle-mediated delivery, exosome-mediated delivery, lipid-nanoparticle-mediated delivery, cell-penetrating-peptide-mediated delivery, or implantable-device-mediated delivery. As specific examples, a nucleic acid or protein can be introduced into a cell or non-human animal in a carrier such as a poly(lactic acid) (PLA) microsphere, a poly(D,L-lactic-coglycolic-acid) (PLGA) microsphere, a liposome, a micelle, an inverse micelle, a lipid cochleate, or a lipid microtubule. Some specific examples of delivery to a non-human animal include hydrodynamic delivery, virus-mediated delivery (e.g., adeno-associated virus (AAV)-mediated delivery), and lipid-nanoparticle-mediated delivery.

Introduction of reagents (e.g., nucleic acids and proteins) into cells or non-human animals can be accomplished by hydrodynamic delivery (HDD). For gene delivery to parenchymal cells, only essential DNA sequences need to be injected via a selected blood vessel, eliminating safety concerns associated with current viral and synthetic vectors. When injected into the bloodstream, DNA is capable of reaching cells in the different tissues accessible to the blood. Hydrodynamic delivery employs the force generated by the rapid injection of a large volume of solution into the incompressible blood in the circulation to overcome the physical barriers of endothelium and cell membranes that prevent large and membrane-impermeable compounds from entering parenchymal cells. In addition to the delivery of DNA, this method is useful for the efficient intracellular delivery of RNA, proteins, and other small compounds in vivo. See, e.g., Bonamassa et al. (2011) Pharm. Res. 28(4):694-701, herein incorporated by reference in its entirety for all purposes.

Introduction of nucleic acids can also be accomplished by virus-mediated delivery, such as AAV-mediated delivery or lentivirus-mediated delivery. Other exemplary viruses/viral vectors include retroviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Exemplary viral titers (e.g., AAV titers) include about 10¹², about 10¹³, about 10¹⁴, about 10¹⁵, and about 10¹⁶ vector genomes/mL. Other exemplary viral titers (e.g., AAV titers) include about 10¹², about 10¹³, about 10¹⁴, about 105, and about 10¹⁶ vector genomes (vg)/kg of body weight.

Introduction of nucleic acids and proteins can also be accomplished by lipid nanoparticle (LNP)-mediated delivery. Lipid formulations can protect biological molecules from degradation while improving their cellular uptake. Lipid nanoparticles are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. An exemplary lipid nanoparticle can comprise a cationic lipid and one or more other components. In one example, the other component can comprise a helper lipid such as cholesterol. In another example, the other components can comprise a helper lipid such as cholesterol and a neutral lipid such as DSPC. In another example, the other components can comprise a helper lipid such as cholesterol, an optional neutral lipid such as DSPC, and a stealth lipid such as S010, S024, S027, S031, or S033.

The LNP may contain one or more or all of the following: (i) a lipid for encapsulation and for endosomal escape; (ii) a neutral lipid for stabilization; (iii) a helper lipid for stabilization; and (iv) a stealth lipid.

Administration in vivo can be by any suitable route including, for example, parenteral, intravenous, oral, subcutaneous, intra-arterial, intracranial, intrathecal, intraperitoneal, topical, intranasal, or intramuscular. Systemic modes of administration include, for example, oral and parenteral routes. Examples of parenteral routes include intravenous, intraarterial, intraosseous, intramuscular, intradermal, subcutaneous, intranasal, and intraperitoneal routes. A specific example is intravenous infusion. Nasal instillation and intravitreal injection are other specific examples. Local modes of administration include, for example, intrathecal, intracerebroventricular, intraparenchymal (e.g., localized intraparenchymal delivery to the striatum (e.g., into the caudate or into the putamen), cerebral cortex, precentral gyrus, hippocampus (e.g., into the dentate gyrus or CA3 region), temporal cortex, amygdala, frontal cortex, thalamus, cerebellum, medulla, hypothalamus, tectum, tegmentum, or substantia nigra), intraocular, intraorbital, subconjuctival, intravitreal, subretinal, and transscleral routes. Significantly smaller amounts of the components (compared with systemic approaches) may exert an effect when administered locally (for example, intraparenchymal or intravitreal) compared to when administered systemically (for example, intravenously). Local modes of administration may also reduce or eliminate the incidence of potentially toxic side effects that may occur when therapeutically effective amounts of a component are administered systemically.

Administration in vivo can be by any suitable route including, for example, parenteral, intravenous, oral, subcutaneous, intra-arterial, intracranial, intrathecal, intraperitoneal, topical, intranasal, or intramuscular. A specific example is intravenous infusion. Compositions can be formulated using one or more physiologically and pharmaceutically acceptable carriers, diluents, excipients or auxiliaries. The formulation can depend on the route of administration chosen. The term “pharmaceutically acceptable” means that the carrier, diluent, excipient, or auxiliary is compatible with the other ingredients of the formulation and not substantially deleterious to the recipient thereof.

The frequency of administration and the number of dosages can depend on the half-life of the delivered proteins or nucleic acids and the route of administration among other factors. The introduction of nucleic acids or proteins into the cell or non-human animal can be performed one time or multiple times over a period of time. For example, the introduction can be performed at least two times over a period of time, at least three times over a period of time, at least four times over a period of time, at least five times over a period of time, at least six times over a period of time, at least seven times over a period of time, at least eight times over a period of time, at least nine times over a period of times, at least ten times over a period of time, at least eleven times, at least twelve times over a period of time, at least thirteen times over a period of time, at least fourteen times over a period of time, at least fifteen times over a period of time, at least sixteen times over a period of time, at least seventeen times over a period of time, at least eighteen times over a period of time, at least nineteen times over a period of time, or at least twenty times over a period of time.

The control non-human animal in the above methods can be the non-human animal prior to treatment with the recombinase. Alternatively, the control non-human animal can be a second non-human animal comprising the reverse-conditional null endogenous lysosomal storage disease gene, wherein the second non-human animal has not been treated with the recombinase. For example, the second non-human animal can be the same age as the non-human animal treated with the recombinase.

The control non-human animal in the above methods can be the non-human animal prior to treatment with the candidate therapeutic agent. Alternatively, the control non-human animal can be a second non-human animal comprising the reverse-conditional null endogenous lysosomal storage disease gene, wherein the second non-human animal has not been treated with the candidate therapeutic agent. For example, the second non-human animal can be the same age as the non-human animal treated with the candidate therapeutic agent.

In some methods, step (b) can further comprise the phenotype of the lysosomal storage disease after a defined time period post-treatment relative to a wild type non-human animal. In one example, the wild type non-human animal is the same age as the non-human animal in step (a). In one example, the wild type non-human animal is the same age as the non-human animal in step (b).

The selected time point after birth can be any suitable time point. For example, the selected time point after birth can be about postnatal day 1 (P1), about P2, about P3, about P4, about P5, about P6, about P7, about P14, about P21, about P28, about 1 month after birth, about 2 months after birth, about 3 months after birth, about 4 months after birth, about 5 months after birth, about 6 months after birth, about 7 months after birth, about 8 months after birth, about 9 months after birth, about 10 months after birth, about 11 months after birth, about 1 year after birth, or later. In a specific example, the selected time point is about P7. In another specific example, the selected time point is about P21. In another specific example, the selected time point is about 2 months after birth.

Likewise, the defined time period post-treatment can be any suitable time period. For example, it can be at least about 1 week, at least about 2 weeks, at least about 3 weeks, at least about 4 weeks, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 1 year, or more after treatment with the recombinase. In a specific example, the defined time period post-treatment is at least about 1 week. In another specific example, the defined time period post-treatment is at least about 1 month. In another specific example, the defined time period post-treatment is at least about 3 months. In another specific example, the defined time period post-treatment is between about 1 month and about 3 months after treatment. In another specific example, the defined time period post-treatment is between about 1 week and about 3 months after treatment.

The lysosomal storage disease phenotype can be any lysosomal storage disease phenotype. Examples of such phenotypes are disclosed elsewhere herein and can be assessed by known methods. In a specific example, the lysosomal storage disease is a mucopolysaccharidosis (i.e., the lysosomal storage disease gene is a mucopolysaccharidosis gene), and the phenotype is a mucopolysaccharidosis phenotype. Examples of such phenotypes are disclosed elsewhere herein and can be assessed by known methods.

In one example, the lysosomal storage disease is mucopolysaccharidosis VI (i.e., the lysosomal storage disease gene is Arsb), and the phenotype is a mucopolysaccharidosis VI phenotype. For example, the phenotype can be a skeletal mucopolysaccharidosis VI phenotype. Examples of such phenotypes are disclosed elsewhere herein and can be assessed by known methods as demonstrated herein. For example, the phenotype can be one of the following relative to a wild type non-human animal: (a) increased accumulation of glycosaminoglycans in the liver; (b) increased accumulation of glycosaminoglycans in the heart; (c) increased accumulation of glycosaminoglycans in the kidney; (d) decreased tibia length; (e) decreased spinal column length; (f) increased tibial growth plate width; and (g) decreased cranial length:width ratio. In one example, the phenotype is increased accumulation of glycosaminoglycans in the liver. In another example, the phenotype is increased accumulation of glycosaminoglycans in the heart. In another example, the phenotype is increased accumulation of glycosaminoglycans in the kidney. In another example, the phenotype is decreased tibia length. In another example, the phenotype is decreased spinal column length. In another example, the phenotype is increased tibial growth plate width. In another example, the phenotype is decreased cranial length:width ratio.

In another example, the lysosomal storage disease is mucopolysaccharidosis I (i.e., the lysosomal storage disease gene is Idua), and the phenotype is a mucopolysaccharidosis I phenotype. Examples of such phenotypes are disclosed elsewhere herein and can be assessed by known methods as demonstrated herein. For example, the phenotype can be a skeletal mucopolysaccharidosis I phenotype or a neurological mucopolysaccharidosis I phenotype. In one example, the phenotype is a skeletal mucopolysaccharidosis I phenotype. In one example, the phenotype is a neurological mucopolysaccharidosis I phenotype.

### B. Methods of Determining Optimal Timeframe for Treatment of a Lysosomal Storage Disease Phenotype

Various methods are provided for determining an optimal timeframe for treatment of a phenotype of the lysosomal storage disease. Such methods can comprise, for example: (a) performing the method of assessing reversibility of a phenotype of the lysosomal storage disease as described above a first time in a first non-human animal comprising a reverse-conditional null endogenous lysosomal storage disease gene, wherein the first non-human animal is treated with the recombinase at a first time point after birth; (b) performing the method of step (a) a second time in a second non-human animal, wherein the second non-human animal is treated with the recombinase at a second time point after birth, wherein the second time point is different from the first time point; and (c) comparing the phenotype in step (a) with the phenotype in step (b) and selecting the time point resulting in the better amelioration of the phenotype.

A phenotype is better ameliorated if the lysosomal storage disease phenotype (e.g., mucopolysaccharidosis phenotype, MIPS VI phenotype, or MIPS I phenotype) is closer to normal (i.e., wild type phenotype). For example, complete rescue of a phenotype (i.e., to wild type levels) is better amelioration of the phenotype than a partial rescue of the phenotype.

The first and second time points after birth can be any suitable time points as described above. In a specific example, the first time point is about postnatal day 7 (P7), and the second time point is about P21. In another specific example, the first time point is about P7, and the second time point is about 2 months after birth. In another specific example, the first time point is about P21, and the second time point is about 2 months after birth. Preferably, the defined time period post-treatment is the same in steps (a) and (b). Examples of suitable time periods post-treatment are disclosed above. In a specific example, the defined time period post-treatment in both steps (a) and (b) is at least about 1 week. In another specific example, the defined time period post-treatment in both steps (a) and (b) is at least about 1 month. In another specific example, the defined time period post-treatment in both steps (a) and (b) is at least about 3 months. In another specific example, the defined time period post-treatment in both steps (a) and (b) is between about 1 month and about 3 months after treatment. In another specific example, the defined time period post-treatment in both steps (a) and (b) is between about 1 week and about 3 months after treatment.

### C. Methods of Assessing Reversibility of a Lysosomal Storage Disease Phenotype by a Combination Therapy

Various methods are provided for assessing the reversibility of a phenotype of a lysosomal storage disease by a combination therapy. Such methods can comprise, for example: (a) performing the method of assessing reversibility of a phenotype of the lysosomal storage disease as described above in a first non-human animal comprising a reverse-conditional null endogenous lysosomal storage disease gene and in a second non-human animal comprising a reverse-conditional null endogenous lysosomal storage disease gene, wherein the first non-human animal is treated with the recombinase but not the candidate therapeutic agent, and wherein the second non-human animal is treated with the recombinase and the candidate therapeutic agent; (b) comparing the phenotype in the first non-human animal with the phenotype in the second non-human animal to determine if the combination of the recombinase and the candidate therapeutic agent results in better amelioration of the phenotype.

A phenotype is better ameliorated if the lysosomal storage disease phenotype (e.g., mucopolysaccharidosis phenotype, MIPS VI phenotype, or MIPS I phenotype) is closer to normal (i.e., wild type phenotype). For example, complete rescue of a phenotype (i.e., to wild type levels) is better amelioration of the phenotype than a partial rescue of the phenotype.

In some methods, the first non-human animal and the second non-human animal are treated with the recombinase at the same time point after birth. In some methods, the second non-human animal is treated with the recombinase and the candidate therapeutic agent simultaneously. In some methods, the second non-human animal is treated with the recombinase and the candidate therapeutic agent sequentially. For example, the second non-human animal can be treated with the recombinase first and then subsequently with the candidate therapeutic agent, or the second non-human animal can be treated with the candidate therapeutic agent first and then subsequently with the recombinase.

The candidate therapeutic agent can be any suitable candidate therapeutic agent as described above. In some methods, the candidate therapeutic agent is not a protein encoded by the endogenous lysosomal storage disease gene or a nucleic acid encoding the protein.

### D. Methods of Assessing Efficacy of a Candidate Therapeutic Agent for Treating a Lysosomal Storage Disease

Various methods are provided for assessing the efficacy of a therapeutic agent for reversing a phenotype of a lysosomal storage disease. Such methods can comprise, for example: (a) performing the method of assessing reversibility of a phenotype of the lysosomal storage disease as described above in a first non-human animal comprising a reverse-conditional null endogenous lysosomal storage disease gene and in a second non-human animal comprising a reverse-conditional null endogenous lysosomal storage disease gene, wherein the first non-human animal is treated with the recombinase but not the candidate therapeutic agent, and wherein the second non-human animal is treated with the candidate therapeutic agent but not the recombinase; (b) comparing the phenotype in the first non-human animal with the phenotype in the second non-human animal to determine if the recombinase results in better amelioration of the phenotype than the candidate therapeutic agent. Such methods can be used to determine the efficacy of the candidate therapeutic agent relative to the maximal effect achieved by turning back on the lysosomal storage disease gene through treating with the recombinase.

A phenotype is better ameliorated if the lysosomal storage disease phenotype (e.g., mucopolysaccharidosis phenotype, MIPS VI phenotype, or MIPS I phenotype) is closer to normal (i.e., wild type phenotype). For example, complete rescue of a phenotype (i.e., to wild type levels) is better amelioration of the phenotype than a partial rescue of the phenotype.

In some methods, the recombinase and the candidate therapeutic agent are administered at the same selected time point after birth. In some methods, the phenotype is assessed after the same defined time period post-treatment in the first non-human animal and the second non-human animal. In some methods, the recombinase and the candidate therapeutic agent are administered at the same selected time point after birth, and the phenotype is assessed after the same defined time period post-treatment in the first non-human animal and the second non-human animal.

The candidate therapeutic agent can be any suitable candidate therapeutic agent as described above. In some methods, the candidate therapeutic agent is a protein encoded by the endogenous lysosomal storage disease gene or a nucleic acid encoding the protein.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### BRIEF DESCRIPTION OF THE SEQUENCES

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. When a nucleotide sequence encoding an amino acid sequence is provided, it is understood that codon degenerate variants thereof that encode the same amino acid sequence are also provided. The amino acid sequences follow the standard convention of beginning at the amino terminus of the sequence and proceeding forward (i.e., from left to right in each line) to the carboxy terminus.

TABLE 4
Description of Sequences.

| SEQ | | |
|---|---|---|
| ID NO | Type | Description |
| 1 | Protein | mARSB (NP_033842.3) |
| 2 | DNA | mArsb cDNA (NM_009712.3) |
| 3 | DNA | mArsb CDS (CCDS36749.1) |
| 4 | Protein | mIDUA (NP_032351.2) |
| 5 | DNA | mIdua cDNA (NM_008325.4) |
| 6 | DNA | mIdua CDS |
| 7 | DNA | MAID8378 (mArsb OFF with |
| | | Neo Self Deleting Cassette) |
| 8 | DNA | MAID8379 (mArsb OFF without |
| | | Neo Self Deleting Cassette) |
| 9 | DNA | MAID8380 (mArsb ON without |
| | | Neo Self Deleting Cassette) |
| 10 | DNA | MAID8375 (mIdua OFF with |
| | | Neo Self Deleting Cassette) |
| 11 | DNA | MAID8376 (mIdua OFF without |
| | | Neo Self Deleting Cassette) |
| 12 | DNA | MAID8377 (mIdua ON, post Cre, |
| | | without Neo Self Deleting Cassette) |
| 13-30 | DNA | Primers and Probes for mArsb Screening |
| 31-46 | DNA | Primers and Probes for mIdua Screening |
| 47 | DNA | Inverted mArsb Sequence |
| 48 | DNA | Reinverted mArsb Sequence |
| 49 | DNA | Inverted mIdua Sequence |
| 50 | DNA | Reinverted mIdua Sequence |
| 51-58 | DNA | Primers and Probes |

### EXAMPLES

### Example 1. Generation of a COIN Mouse Model of Mucopolysaccharidosis VI

Mucopolysaccharidosis VI (MPS VI), also known as Maroteaux-Lamy Syndrome, is a lysosomal disease resulting from impaired function of the arylsulfatase B (ARSB) protein. This impairment causes aberrant accumulation of dermatan sulfate, a glycosaminoglycan (GAG) abundant in growth plates, cartilage, and extracellular matrix. While clinical presentation is variable in terms of age at first symptom manifestation and disease severity, MPS VI classically presents at early ages and significantly impacts the skeleton. Current treatment guidelines recommend enzyme replacement therapy (ERT), which is known to provide incomplete or ineffective recovery from the skeletal manifestations of disease. We can postulate this may be due to the inability of the exogenous enzyme to reach affected cells, or that disease may not be reversible at the time therapy is delivered. To date, no models of disease exist that separate treatment efficacy from disease reversibility. To date, no model exists where efficacy of ERT and reversibility of the phenotype can be explored independently.

To this end, we have generated a “conditional-on” mouse model of MPS VI, using a Rosa Cre-ERT2 plus floxed-inversion approach (COIN) (i.e., a conditional by inversion (COIN) mouse model of MPS VI, Arsb^{COIN/COIN}). In this design, ARSB expression is initially “off” due to the inversion of exon 5 in the Arsb gene. In order to restore ARSB expression at different times during postnatal development, we introduced a tamoxifen-dependent global Cre driver, Gt(ROSA26)Sor^{CreERt2/+}. After tamoxifen administration, exon 5 is restored to its native orientation, enabling ARSB expression. This approach allows us to restore endogenous expression of the missing enzyme starting at different points during disease progression, and therefore define the window of reversibility. This approach allowed us to turn back “on” endogenous expression of the missing enzyme at several points during disease progression. Probing gene rescue in adult mice, at age P21, and at age P7, we have determined that the skeletal phenotype can be rescued at with intervention at P7, but efficacy declines with further age. By restoring Arsb expression at postnatal days 7, 21, and 56-70 (P7, P21, and P56-P70), we determined long bone length and other skeletal phenotypes can be fully rescued if gene restoration occurs at P7, while only partial rescue occurs with later interventions.

A large targeting vector (LTVEC) comprising a 5′ homology arm comprising 81 kb of the mouse Arsb locus and 3′ homology arm comprising 102 kb of the mouse Arsb locus was generated to replace a region surrounding exon 5 with a corresponding inverted region flanked by a 5′ lox71 site and a 3′ lox66. A roxed self-deleting cassette was inserted downstream of the inverted sequence. Information on the mouse Arsb gene is provided in Table 5. A description of the generation of the large targeting vector is provided in Table 6. Generation and use of large targeting vectors (LTVECs) derived from bacterial artificial chromosome (BAC) DNA through bacterial homologous recombination (BHR) reactions using VELOCIGENE® genetic engineering technology is described, e.g., in U.S. Pat. No. 6,586,251 and Valenzuela et al. (2003) Nat. Biotechnol. 21(6):652-659, each of which is herein incorporated by reference in its entirety for all purposes. Generation of LTVECs through in vitro assembly methods is described, e.g., in US 2015/0376628 and WO 2015/200334, each of which is herein incorporated by reference in its entirety for all purposes.

TABLE 5
Mouse Arsb.

| | Gene | | RefSeq | | Genomic | |
|---|---|---|---|---|---|---|
| | Symbol | NCBI Gene ID | mRNA ID | UniProt ID | Assembly | Chromosomal Location |
| Mouse | Arsb | 11881 | NM_009712 | P50429 | GRCm38/ | chr13:93,771,630- |
| | | | | | mm10 | 93,943,016 (+) |

TABLE 6
Mouse Arsb Large Targeting Vector.

| | | | | Length |
|---|---|---|---|---|
| | Genome Build | Start | End | (bp) |
| 5′ Mouse Arm | GRCm38/mm10 | chr13: | chr13: | 81168 |
| | | 93,780,418 | 93,861,585 | |
| Human Insert | GRCm38/mm10 | chr13: | chr13: | 1127 |
| | | 93,861,605 | 93,862,731 | |
| 3′ Mouse Arm | GRCm38/mm10 | chr13: | chr13: | 101980 |
| | | 93,862,755 | 93,964,734 | |

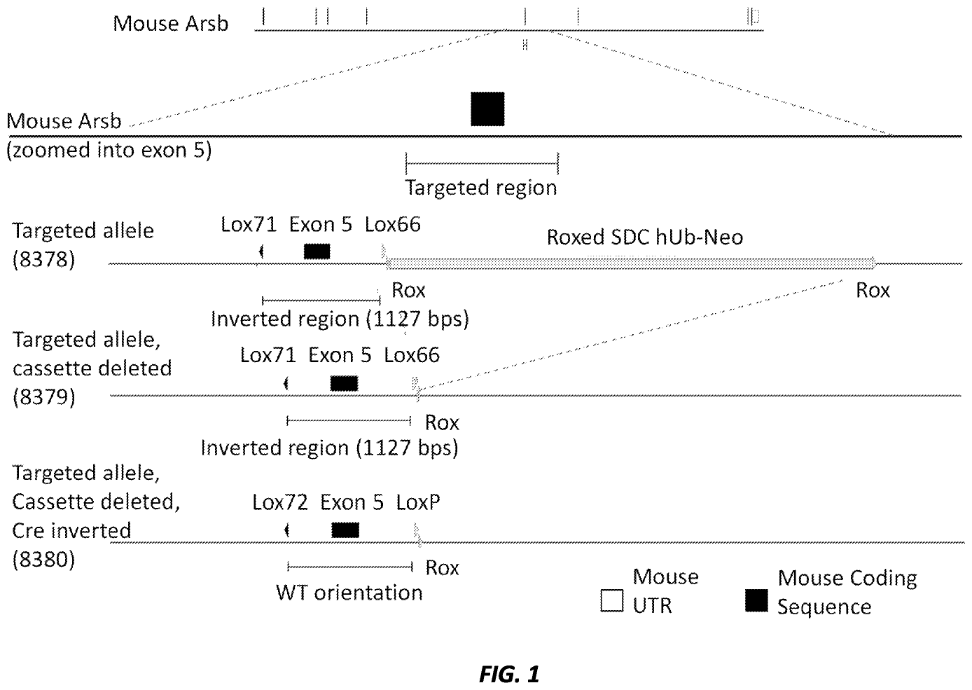

Specifically, a region surrounding exon 5 (coding exon 5; amino acids 301-382) of the Arsb gene, including 482 bps of the 5′ intron and 401 bps of the 3′ intron, was inverted in the mouse Arsb locus. A 19 bp deletion immediately 5′ and a 23 bp deletion immediately 3′ of this region were incorporated to facilitate screening in mouse embryonic stem cells. The inverted region (SEQ ID NO: 47) is flanked by a 5′ lox71 site and a 3′ lox66. A roxed self-deleting cassette was inserted 3′ of the inverted region. This is the MAID 8378 allele (SEQ ID NO: 7). The allele after cassette deletion is the MAID 8379 allele (SEQ ID NO: 8). The cassette-deleted allele after rescue by inversion of the inverted exon 5 region (reinverted region set forth in SEQ ID NO: 48) is the MAID 8380 allele (SEQ ID NO: 9). See FIG. 1.

The expected encoded ARSB protein after rescue by inversion of the inverted exon 5 region is a fully mouse ARSB protein. See FIG. 1. The mouse ARSB protein and Arsb coding sequences are set forth in SEQ ID NOS: 1 and 3, respectively.

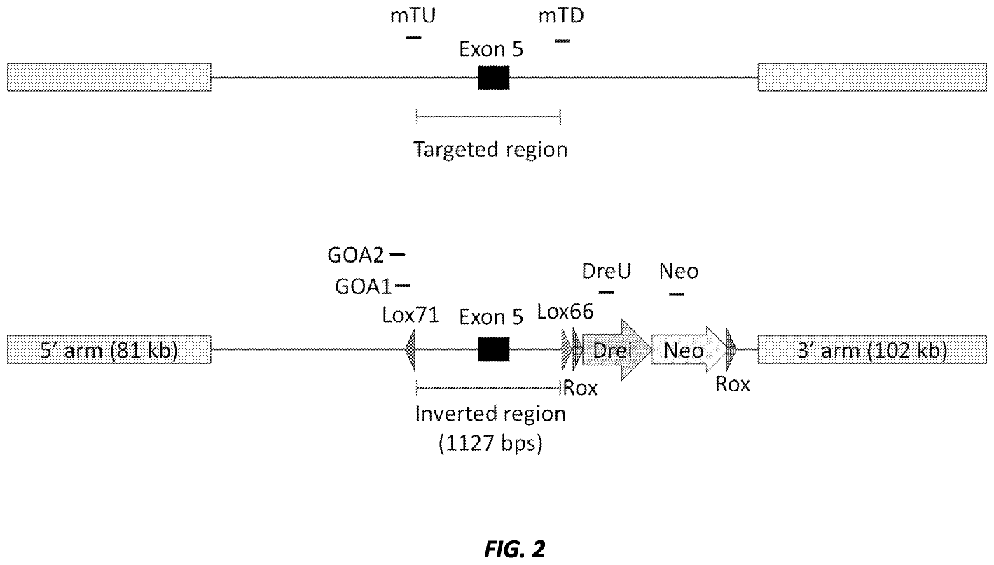

To generate the mutant allele a large targeting vector was introduced into mouse embryonic stem cells. Specifically, 2×10⁶ mouse VGF1 embryonic cells (50% C57BL/6NTac 50% 129S6/SvEvTac) carrying a Rosa26-CreER^{T2} allele were electroporated with 0.4 μg mArsb LTVEC. The electroporation conditions were: 400 V voltage; 100 mF capacitance; and 0 W resistance. Antibiotic selection was performed using G418 at a concentration of 100 mg/mL. Colonies were picked, expanded, and screened by TAQMAN®. See FIG. 2. Loss-of-allele assays were performed to detect loss of the endogenous mouse allele, and gain-of-allele assays were performed using the primers and probes set forth in Table 7. The GOA2 gain-of-allele assays was used to detect the mArsb gene inversion, after tamoxifen-Cre activation, using the primers and probes set forth in Table 7.

TABLE 7
Screening Assays.

| | | | | SEQ |
|---|---|---|---|---|
| | | | | ID |
| Assay | Description | Primer/Probe | Sequence | NO |
| 8378mTU | Upstream | Fwd | TGAAGCAATAACCCTTGACCTAAA | 13 |
| | LOA | Probe (FAM) | TGAGAAGCAGGAACAGCACTAGGCC | 14 |
| | | Rev | TGCCAGGACGTCTATCCAA | 15 |
| 8378mTD | Downstream | Fwd | AATGTCTGTGGTTCGGTACCTAG | 16 |
| | LOA | Probe (Cal-Orange) | TCACATGTACTGGAAGCAAATGACTATTGC | 17 |
| | | Rev | GCACACCAAAGAGGCTGAAAG | 18 |
| 8378GOA | GOA1- | Fwd | AGGCCAAGATTGACAGTTACCAG | 19 |
| | Upstream | Probe (FAM) | ATGCAGAAACTCGAGGGATCTCCTACC | 20 |
| | Insertion | Rev | GGAGTACAGGGAAGGAAACCT | 21 |
| 2154MUP | GOA2- | Fwd | CCAGCCAGAATTTATATGCAGAAA | 22 |
| | Cre | Probe (Cal) | CTCGAGGGATCTCC | 23 |
| | Activation | Rev | CAGTCATAGCTGTCCCTCTTCTCTT | 24 |
| DreU | Selection | Fwd | GCCTCCGCTGTCTGACGAT | 25 |
| | Cassette | Probe (FAM) | AAGTCTGTGTCCCTTGCCATGCG | 26 |
| | Assay | Rev | GCTGCCTCCCGTCTGATTC | 27 |
| Neo | Selection | Fwd | GGTGGAGAGGCTATTCGGC | 28 |
| | Cassette | Probe (Cal) | TGGGCACAACAGACAATCGGCTG | 29 |
| | Assay | Rev | GAACACGGCGGCATCAG | 30 |

Modification-of-allele (MOA) assays including loss-of-allele (LOA) and gain-of-allele (GOA) assays are described, for example, in US 2014/0178879; US 2016/0145646; WO 2016/081923; and Frendewey et al. (2010) Methods Enzymol. 476:295-307, each of which is herein incorporated by reference in its entirety for all purposes. The loss-of-allele (LOA) assay inverts the conventional screening logic and quantifies the number of copies in a genomic DNA sample of the native locus to which the mutation was directed. In a correctly targeted heterozygous cell clone, the LOA assay detects one of the two native alleles (for genes not on the X or Y chromosome), the other allele being disrupted by the targeted modification. The same principle can be applied in reverse as a gain-of-allele (GOA) assay to quantify the copy number of the inserted targeting vector in a genomic DNA sample.

F0 mice were generated from the modified ES cells using the VELOCIMOUSE® method. Specifically, mouse ES cell clones comprising the modified Arsb locus described above that were selected by the MOA assay described above were injected into 8-cell stage embryos using the VELOCIMOUSE® method. See, e.g., U.S. Pat. Nos. 7,576,259; 7,659,442; 7,294,754; US 2008/0078000; and Poueymirou et al. (2007) Nat. Biotechnol. 25(1):91-99, each of which is herein incorporated by reference in its entirety for all purposes. In the VELOCIMOUSE® method, targeted mouse ES cells are injected through laser-assisted injection into pre-morula stage embryos, e.g., eight-cell-stage embryos, which efficiently yields F0 generation mice that are fully ES-cell-derived. In the VELOCIMOUSE® method, the injected pre-morula stage embryos are cultured to the blastocyst stage, and the blastocyst-stage embryos are introduced into and gestated in surrogate mothers to produce the F0 generation mice. When starting with mouse ES cell clones homozygous for the targeted modification, F0 mice homozygous for the targeted modification are produced. When starting with mouse ES cell clones heterozygous for the targeted modification, subsequent breeding can be performed to produce mice homozygous for the targeted modification.

### Example 2. Use of COIN Model in MPS VI to Define Phenotype Reversibility

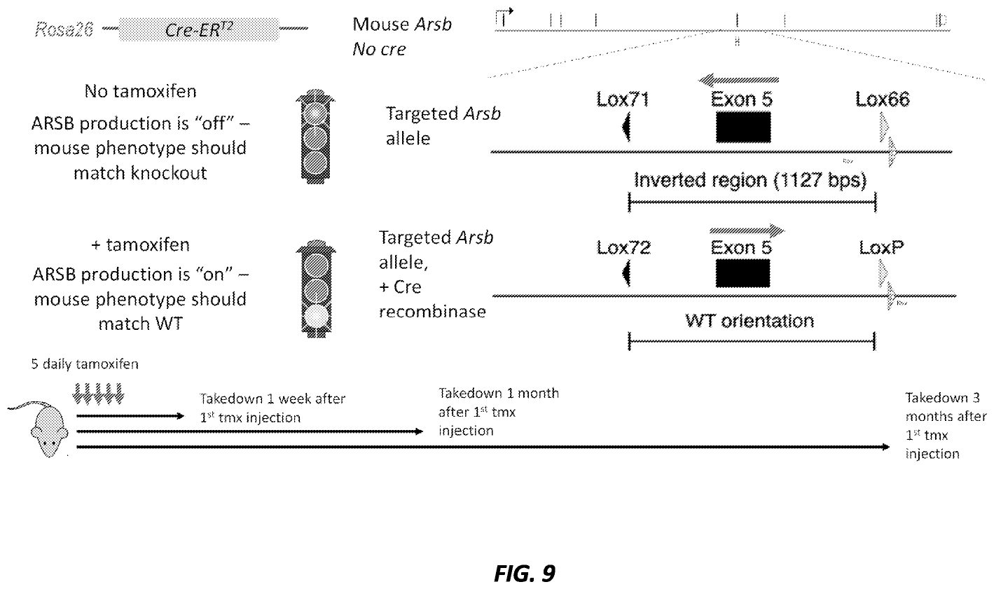

The mouse model generated in Example 1 allows restoration of Arsb expression from the native locus at desired timepoints, bypassing the limitations of enzyme replacement therapy (ERT). In the absence of tamoxifen, ARSB production is “off,” replicating the Arsb knockout phenotype, because the region surrounding exon 5 is inverted. In the presence of tamoxifen, expression of Cre recombinase is induced, and the Cre recombinase inverts the inverted exon 5 region, flipping it back into the correct orientation, restoring wild type Arsb expression. See FIG. 9.

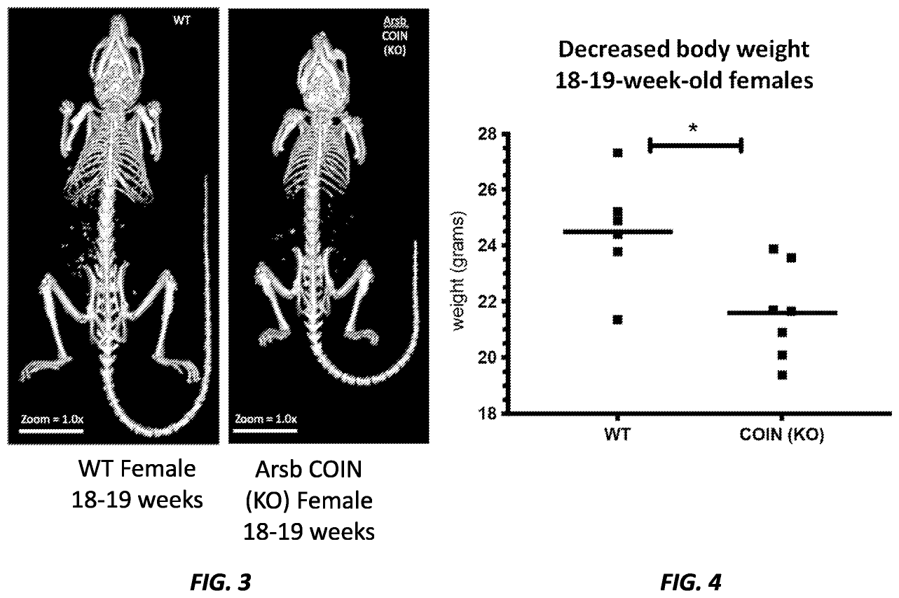

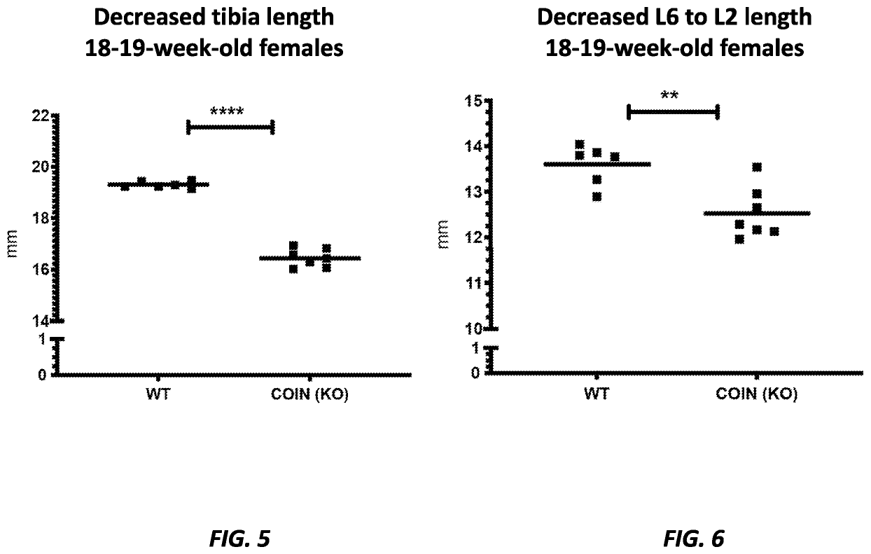

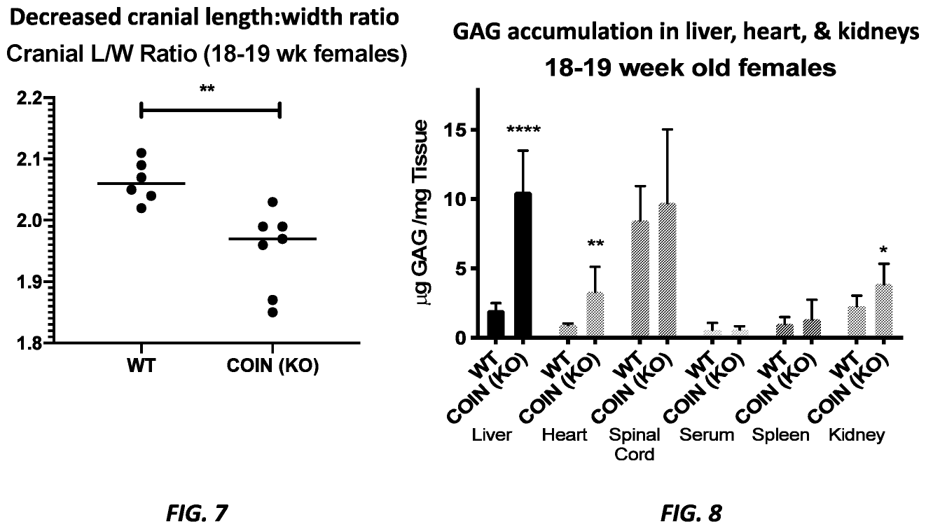

The COIN Arsb mice were first assessed in the absence of tamoxifen to assess whether they display key features of MPS VI. COIN Arsb mice were examined alongside an age and sex matched wild type (WT) cohort for skeletal and peripheral tissue phenotypes. These mice were anesthetized prior to micro computerized tomography (μCT) scans. Two days later they were CO₂-euthanized and liver, heart, spinal cord, spleen, kidney, and sera were harvested. Total sulfated GAG in each tissue type was assessed using dimethylmethylene blue (DMMB), which is a chromogen that presents an absorption change upon binding the sulfated moiety in GAGs. Similar to Arsb knockout mice, the COIN Arsb mice without tamoxifen treatment showed decreased body weight, decreased tibia lengths, decreased spinal column lengths, increased tibial growth plate width, decreased cranial length:width ratio, and increased GAG accumulation in liver, heart, and kidneys compared to wild type mice. See FIGS. 3-8. Total sulfated GAGs were found to be significantly elevated in liver, heart, and kidney of Arsb^{COIN/COIN} mice. In addition, these mice weighed significantly less. Tomography revealed a difference in overall skeletal size, with long bones (using tibia lengths as representative) of Arsb^{COIN/COIN} mice remarkably shorter. Reduced spinal cord length and coarse facies were also seen, with L6 to L2 lengths significantly shorter, as were cranial length to width ratios.

To assess phenotype reversibility at different timepoints, the COIN Arsb mice described in Example 1 were treated with 5 daily tamoxifen injections starting at different timepoints: 7 days after birth (P7), 21 days after birth (P21), or 8-10 weeks after birth. For the P7 time point, 1 mg tamoxifen per 25 g body weight was injected. For other time points, 2 mg tamoxifen per 25 g body weight was injected. Mice were taken down at 1 week, 1 month or 3 months after tamoxifen injection. See FIG. 9.

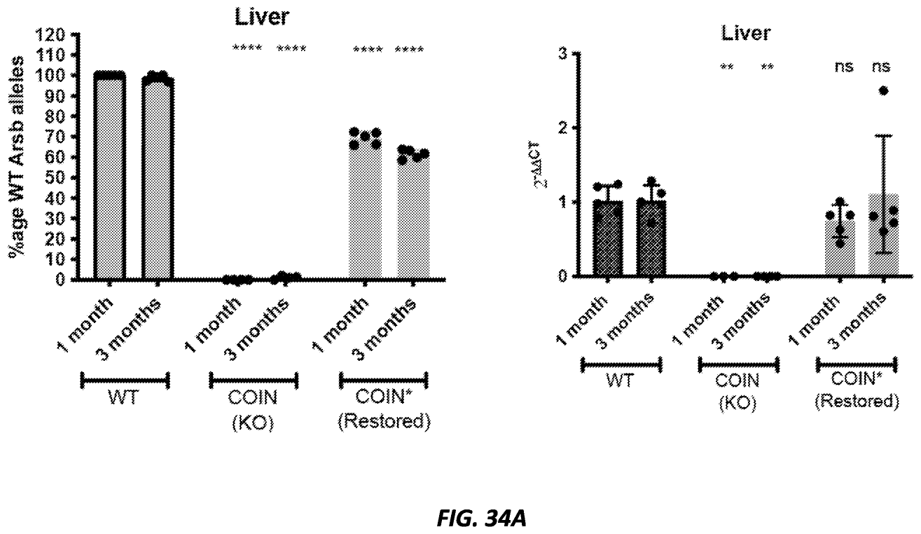

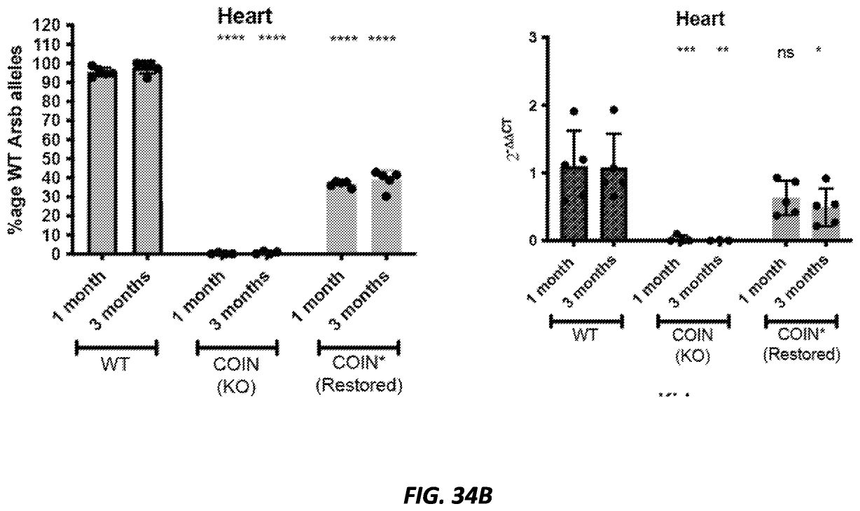

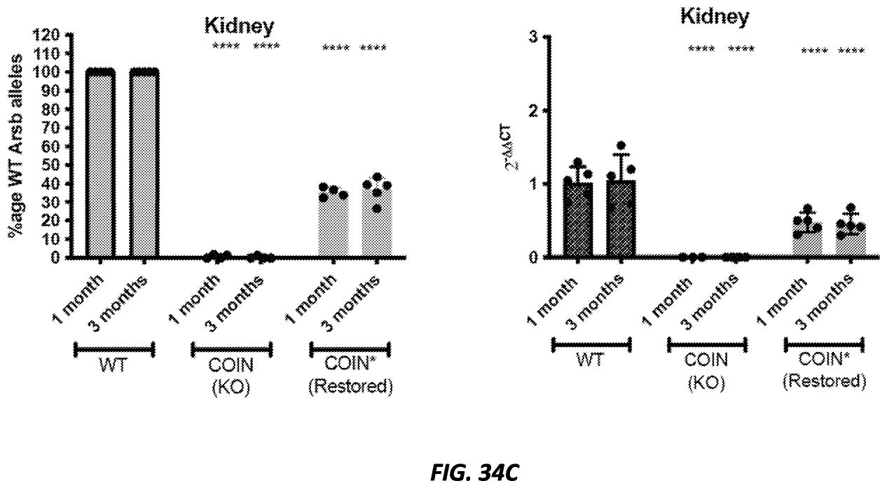

In order to evaluate percentage of inversion back to the WT orientation of the flipped exon in Arsb, we performed recombination assays using genomic DNA extracted from affected peripheral organs. Following tamoxifen treatment in adult COIN Arsb mice, recombination assays indicated that conversion from the inverted allele back to rescued allele remains stable over time in soft tissue. The gene conversion from the inverted allele to the rescued allele occurred at a rate of ˜60%-80% in the liver and spleen and at a rate of ˜30%-40% in the heart and kidney (as measured by qPCR) following treatment with tamoxifen (FIGS. 34A-34C). No inversion was shown in Cre negative control samples. To confirm that the recombined allele (or the rescued, or post-inversion allele) resulted in a functional Arsb, we performed RT-qPCR to check for presence of Arsb mRNA. Transcript abundance in livers was rescued to WT levels by 3 months post injection regardless of which age the restoration of Arsb was initiated. Transcript levels in the heart and kidney ranged from 20 to 70% of WT when restoration occurred at P56-P70 or P21. All tissue types with treatment at P7 exhibited rescue of Arsb mRNA, most to WT levels. No transcript was detected in Cre negative control samples, where the exon would presumably remain inverted (FIGS. 34A-34C).

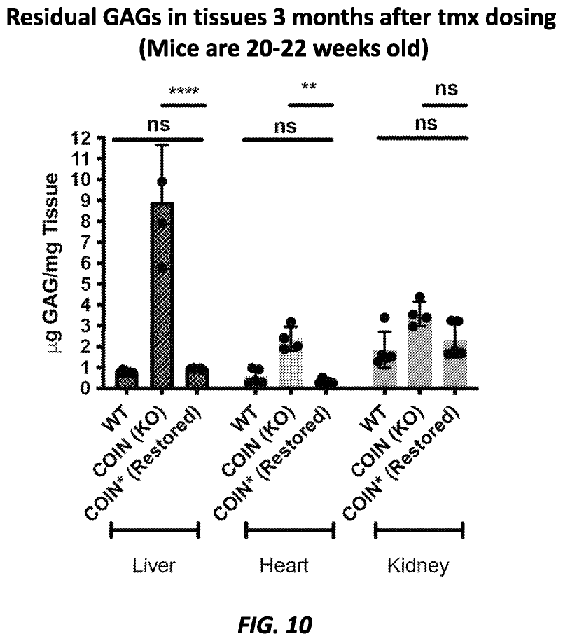

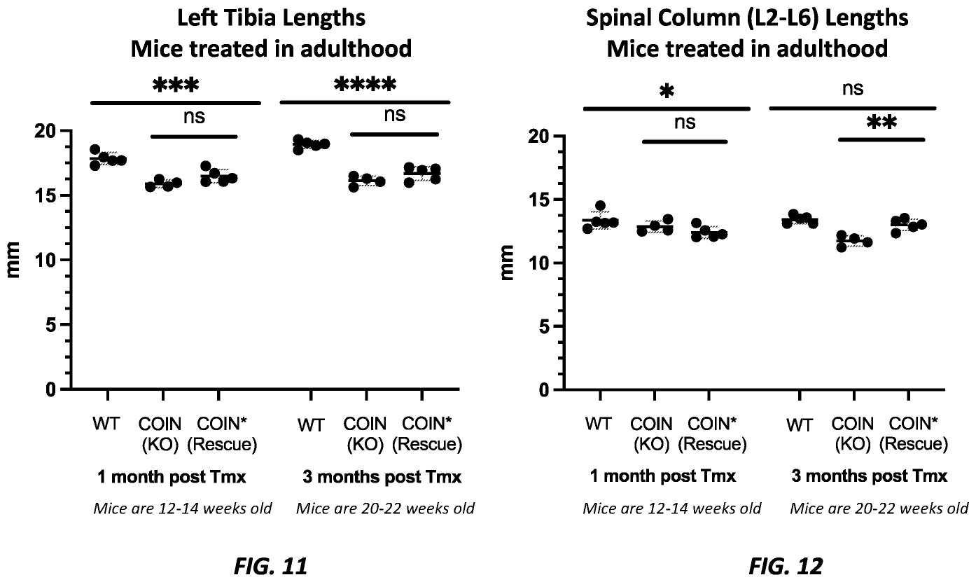

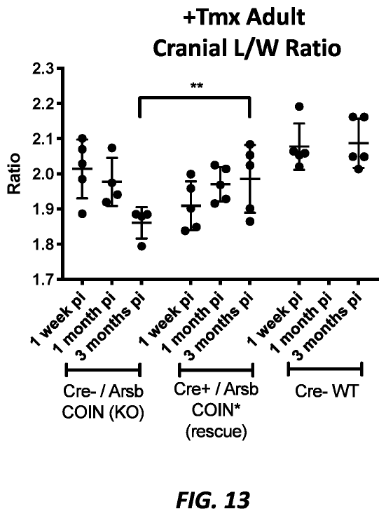

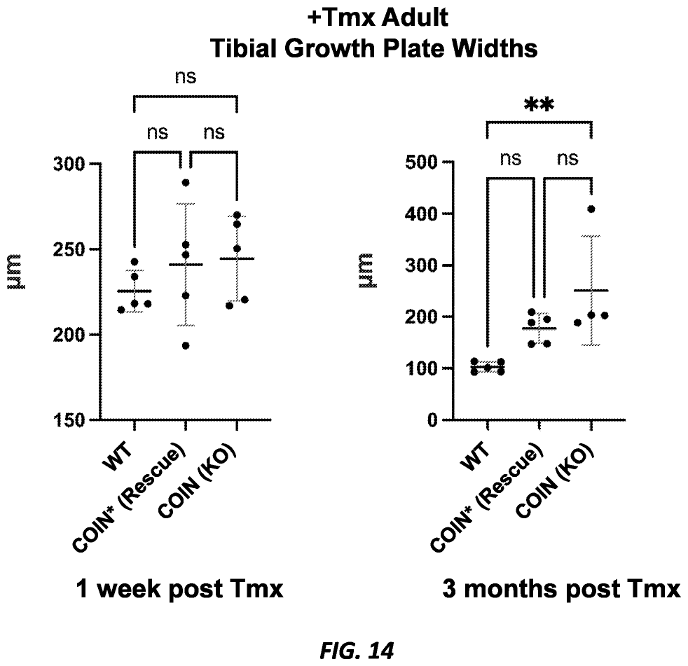

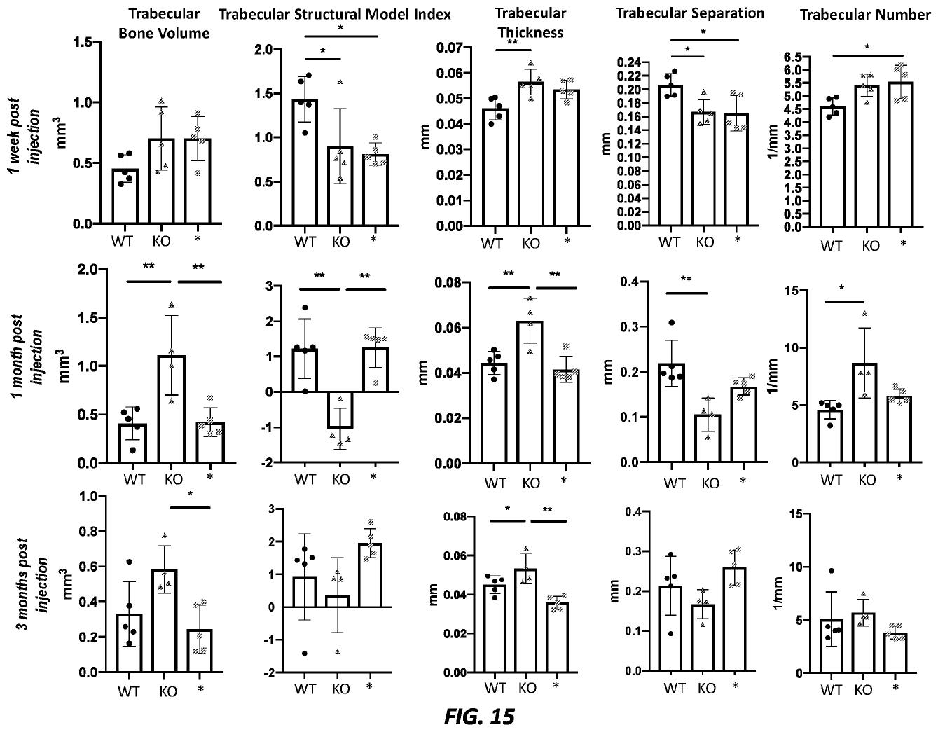

Restoring Arsb expression in the adult mice (injected with tamoxifen 8-10 weeks after birth) reduced glycosaminoglycans (GAGs) in the liver and heart, but had minimal effect on bone lengths. Specifically, mice were dosed with tamoxifen beginning at 8-10 weeks of age according to the dosing paradigm described above. We first sought to assess rescue if GAG accumulation. As shown in FIG. 10, restoration of Arsb at its native locus in adulthood cleared residual GAGs in the liver and heart at 3-months post-tamoxifen injection, with a trend towards reduced residual GAGs in the kidney. Similar reduction in GAG accumulation was observed at 1 month post tamoxifen injection (data not shown). To determine whether restoration of Arsb to the WT state can rescue skeletal growth, we utilized left tibia length as a surrogate measurement. In addition, spinal column length and cranial length to width ratios were determined using μCT. However, as shown in FIG. 11, minimal effects on bone length were seen (the KO control mice exhibited significantly shorter tibia lengths, which were not corrected at either 1 month or 3 months post-tamoxifen treatment in the restoration group), although there was a small but significant increase in the spinal column length (FIG. 12) (there was a moderate, progressive shortening of the spinal column in the KO cohort, which was not corrected by 1 month post-treatment in the restoration group but seemed to be corrected by 3 months post-treatment). Similarly, there were minimal effects on cranial length-to-width ratio, a commonly used method to quantify changes in skull and facial morphology, although there was a significant increase at 3 months post-injection (FIG. 13). Tibial growth plates from tamoxifen-treated cohorts were sectioned and processed with the GAG-specific Alcian Blue stain at 3 months post treatment. Restoration of Arsb in adulthood may not significantly improve the tibial growth plate width phenotype (FIG. 14). In addition, vacuolation in the growth plate was not fully corrected total vacuolated area normalized to growth plate area was reduced, but did not approach WT levels (data not shown). However, high resolution uCT scans of tamoxifen-treated Arsb rescue allele femurs showed that trabecular bone parameters showed significant improvement by 1 month post-tamoxifen treatment in adult mice (FIG. 15). Left femurs from tamoxifen-treated cohorts were extracted, fixed, and μCT scanned ex vivo under high resolution. Trabecular bone, located sub-proximal to growth plates in long bones, is significantly remodeled post differentiation. Consistent with previous models, Arsb COIN (KO) mice displayed increased density in the trabecular space, with significantly increased trabecular bone volume, number of trabeculae, trabecular thickness, and less separation. These parameters correlated with a decrease in structural model indices. Midshaft, mature cortical bone displayed a similar phenotype, with significantly increased cortical bone volume, cortical thickness, and total cortical bone area fraction. Cortical parameters also correlated with differences in shape parameters, e.g. the increase observed in cortical polar moment of inertia. With both trabecular and cortical readouts, restoration of Arsb rescued phenotypes to WT levels.

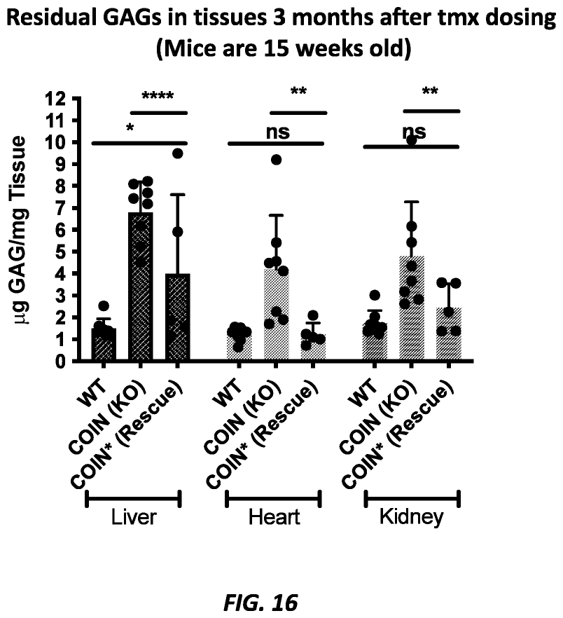

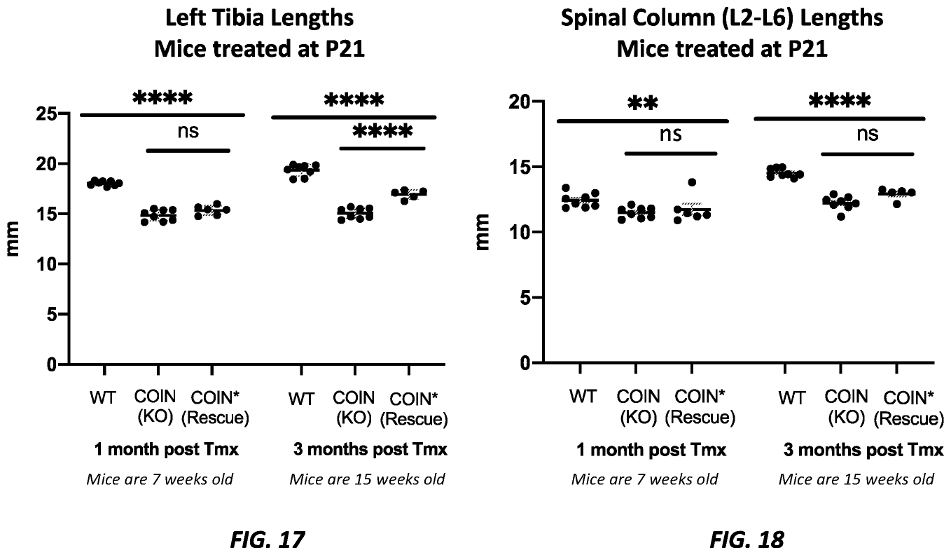

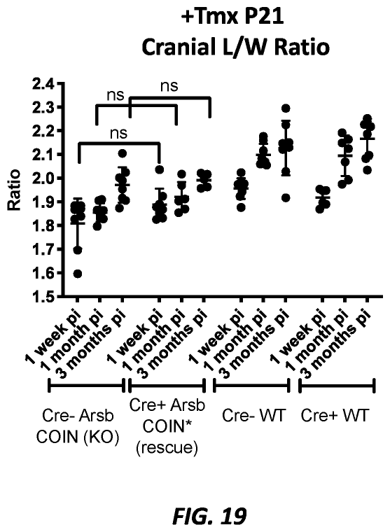

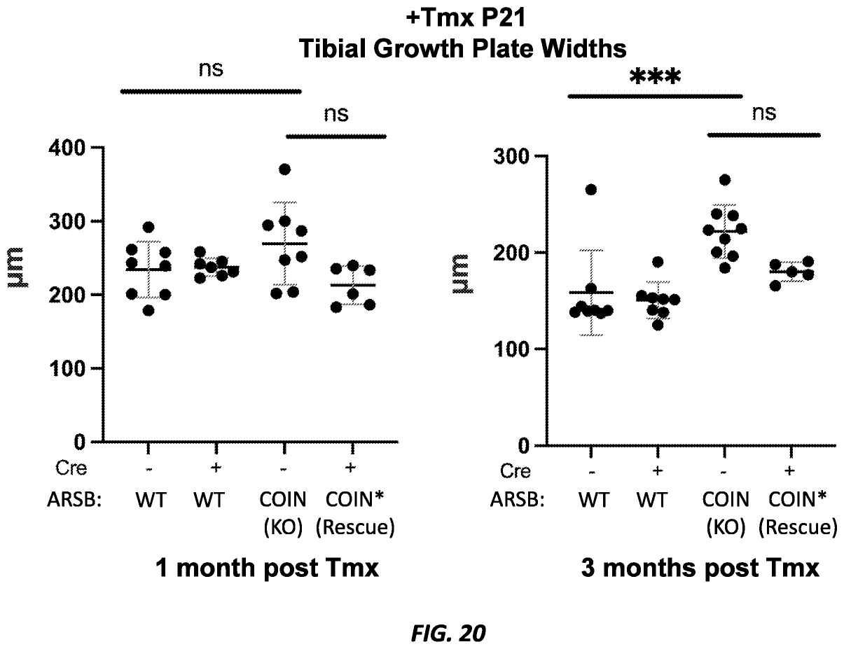

Restoring Arsb expression at P21 partially rescued long bone and spinal column length. Specifically, mice were dosed with tamoxifen at post-natal day 21 (P21) according to the dosing paradigm described above. Similar to observations in older mice, restoring Arsb expression at P21 reduced residual GAGs in the liver, heart, and kidney. See FIG. 16. As shown in FIG. 17, tibial lengths showed significant improvement by 1 month post-tamoxifen, and further improvement by 3 months after restoring Arsb expression, yet fail to reach WT lengths. However, the rescue of bone length was significantly reduced compared to restoration of Arsb expression at P7 (see below), suggesting that the treatment window is already closing by P21 for skeletal phenotypes. In addition, there was a trend towards increased spinal column lengths in treated mice, but the difference was not significant (FIG. 18). When restoration is initiated at P21, there was an increasingly significant difference in L2 to L6 lengths from 1 to 3 months post treatment, but even at 3 months post-restoration spinal column length failed to reach WT levels. Likewise, there was not a significant difference in cranial length-to-width ratio between Arsb KO and Arsb rescue mice 1 week, 1 month, or 3 months after dosing at P21 (FIG. 19), and there was not a significant difference in tibial growth plate width between Arsb KO and Arsb rescue mice 1 month or 3 months after dosing at P21 (FIG. 20). Vacuolation in the growth plate was not fully corrected when restoration is initiated at any age (data not shown). Total vacuolated area normalized to growth plate area was reduced in the restoration group but did not approach WT levels (data not shown).

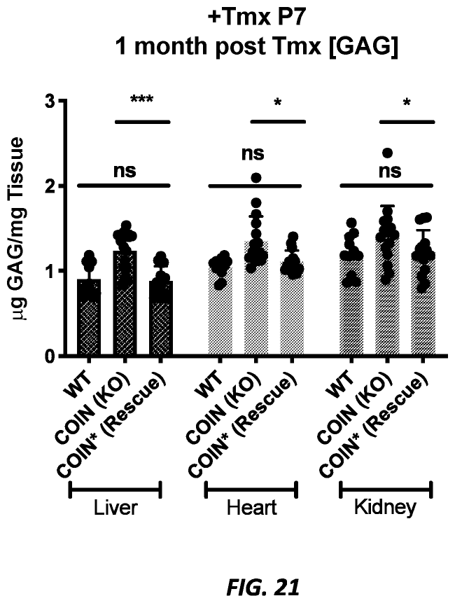

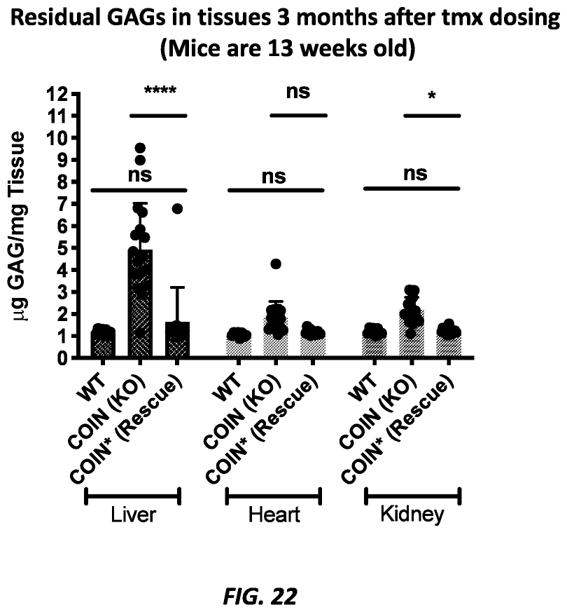

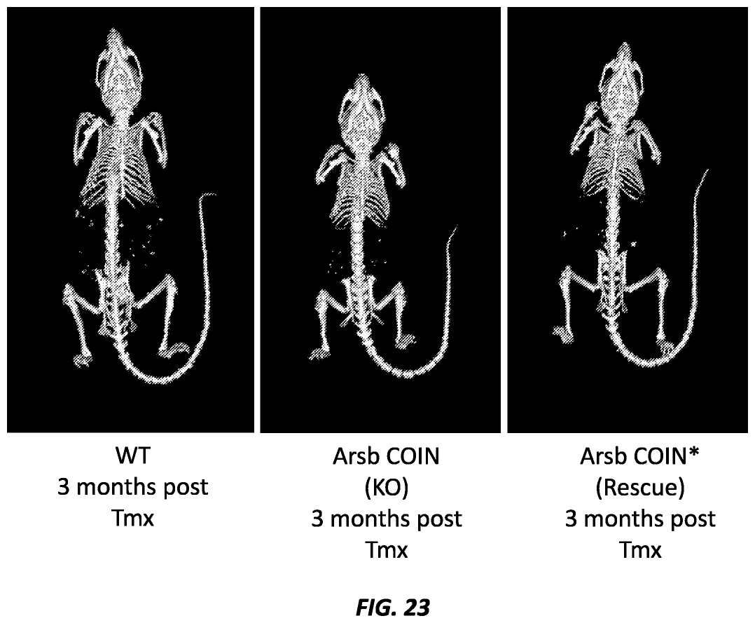

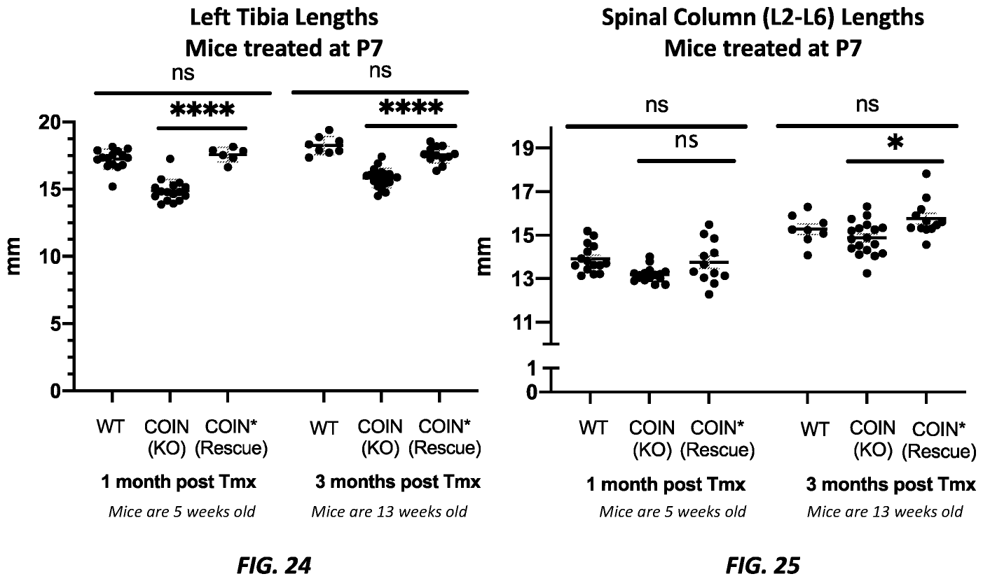

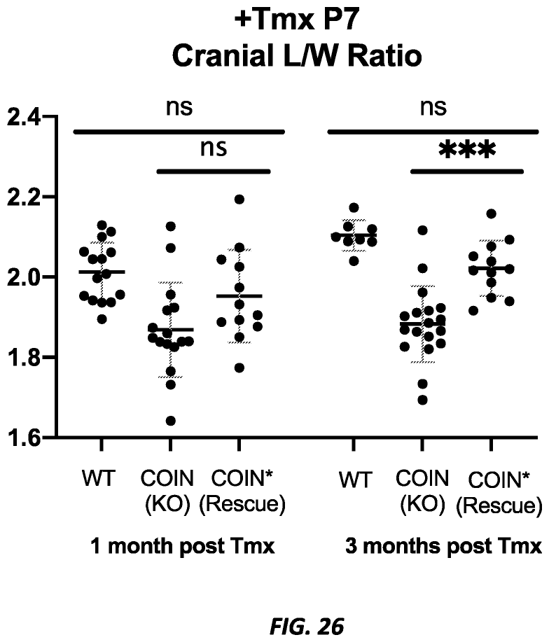

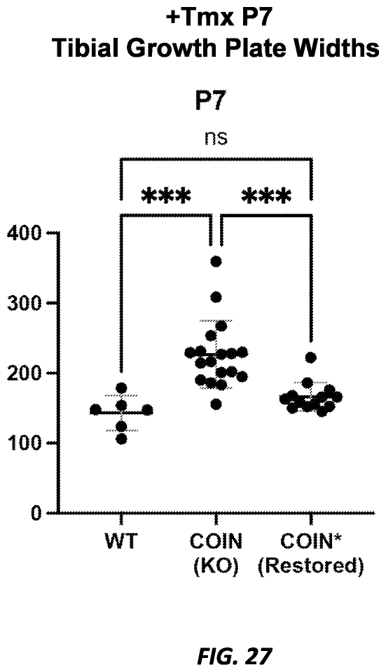

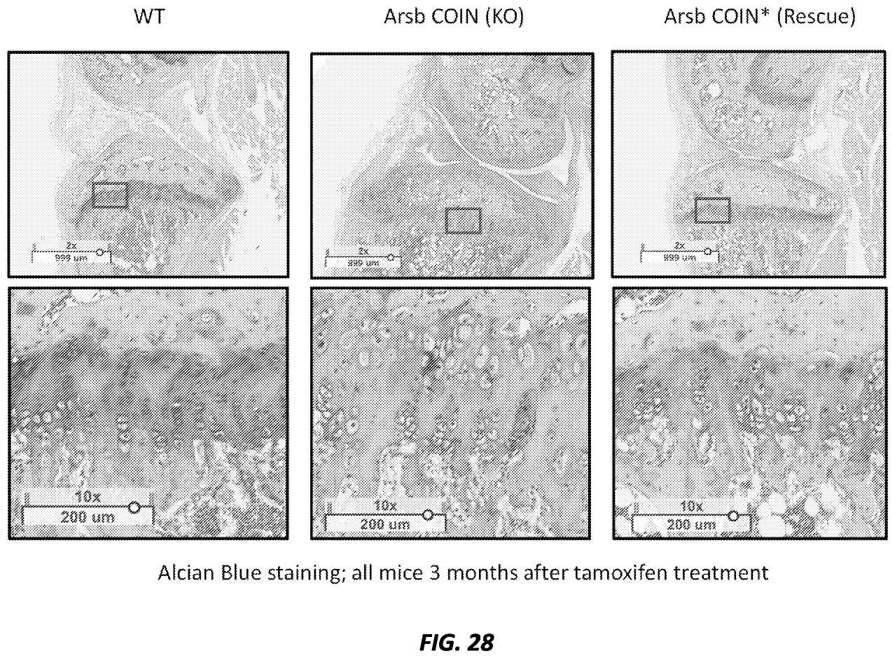

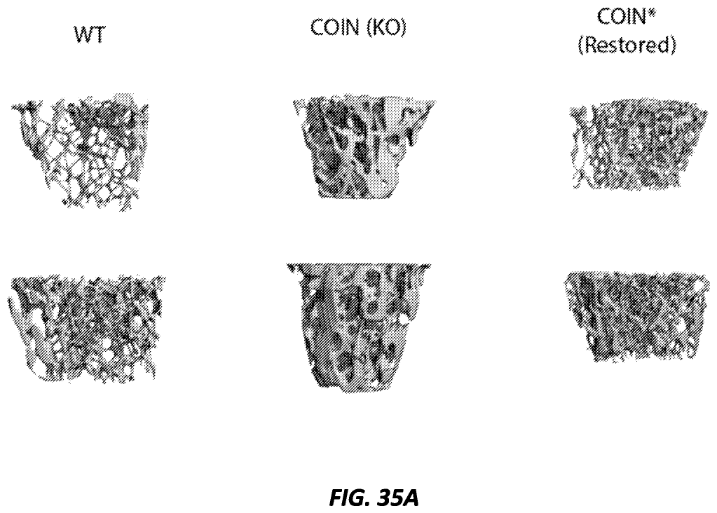

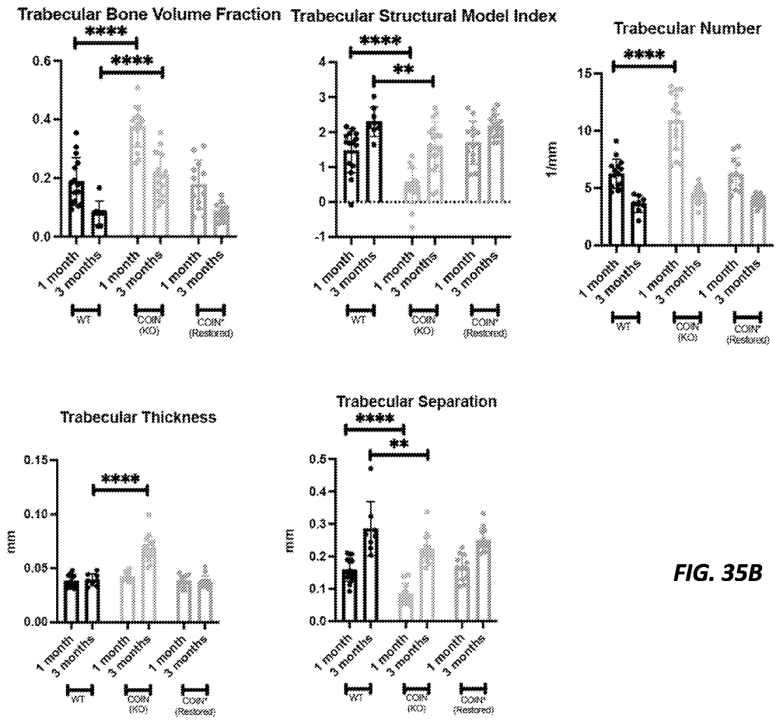

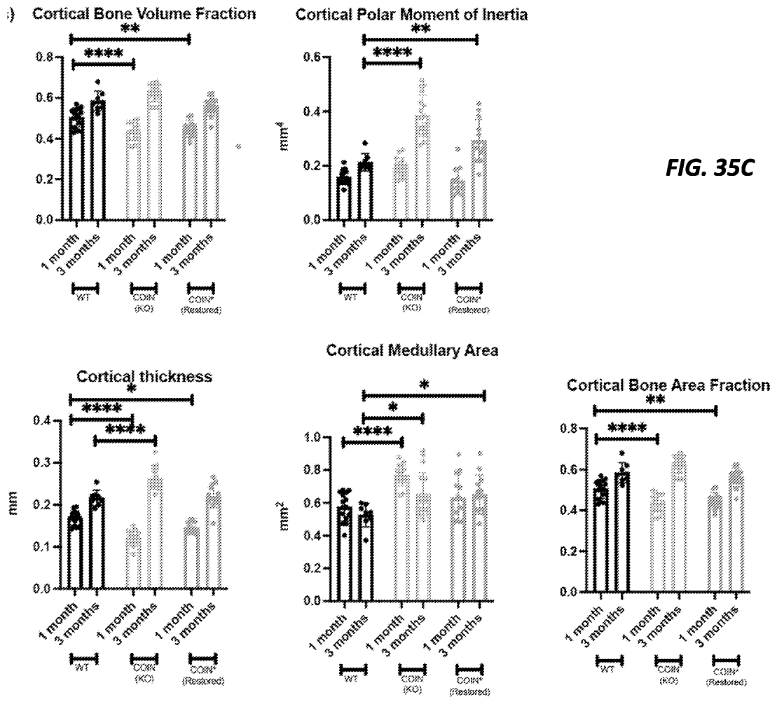

Restoring Arsb expression at P7 rescued long bone and spinal cord lengths to comparable to wild-type mice. Specifically, mice were dosed with tamoxifen at post-natal day 7 (P7) according to the dosing paradigm described above. Similar to observations in older mice, restoring Arsb expression at P7 reduced residual GAGs in the liver, heart, and kidney at both 1 month and 3 months post-tamoxifen treatment. See FIG. 21 and FIG. 22, respectively. Unlike older mice, tibia lengths were rescued to near-wild type lengths by 1 month post-tamoxifen injection. When Arsb was restored starting at P7, there was full correction of the skeletal length phenotype to WT levels at both 1 and 3 months post-treatment, whereas the diseased, untreated group tibia lengths remain significantly shorter. See FIG. 23 and FIG. 24. Spinal column length and cranial length to width ratios were determined using μCT. When restoration was initiated at P7, we observe full rescue to WT lengths by 3 months post treatment. Mirroring the correction observed in the growth of the post-cranial skeleton, strongly significant effect was seen at 3 months post treatment in the P7 restored cohort, at which cranial length to width ratios are corrected to WT levels. These differences were striking in dorsal radiographs from representative μCT images from each group (data not shown). Spinal column length differences (FIG. 25) and cranial length:width ratios (FIG. 26) between groups were not detected at 1 month post-tamoxifen injections. At 3 months, there was a significant increase in spinal column length and cranial length:width ratio, with both being rescued in treated mice. See FIG. 25 and FIG. 26, respectively. Tibial growth plate widths were rescued to near-wild type lengths by 3 months post-tamoxifen injection. See FIG. 27 and FIG. 28. Vacuolation in the growth plate was not fully corrected with restoration initiated at P7 (data not shown). Total vacuolated area normalized to growth plate area was reduced in the restoration group but did not approach WT levels (data not shown). Left femurs from P7 tamoxifen-treated cohorts were extracted, fixed, and μCT scanned ex vivo under high resolution. Trabecular bone, located sub-proximal to growth plates in long bones, is significantly remodeled post differentiation. Arsb COIN (KO) mice display increased density in the trabecular space (FIG. 35A), with significantly increased trabecular bone volume, number of trabeculae, trabecular thickness, and less separation (FIG. 35B). These parameters correlate with a decrease in structural model indices. Midshaft, mature cortical bone displays a similar phenotype, with significantly increased cortical bone volume, cortical thickness, and total cortical bone area fraction (FIG. 35C). Cortical parameters also correlate with differences in shape parameters, e.g. the increase observed in cortical polar moment of inertia. With both trabecular and cortical readouts, restoration of Arsb rescues phenotypes to WT levels (FIGS. 35A-35C). Analyses performed in adult mice exhibited largely similar results (FIG. 15).

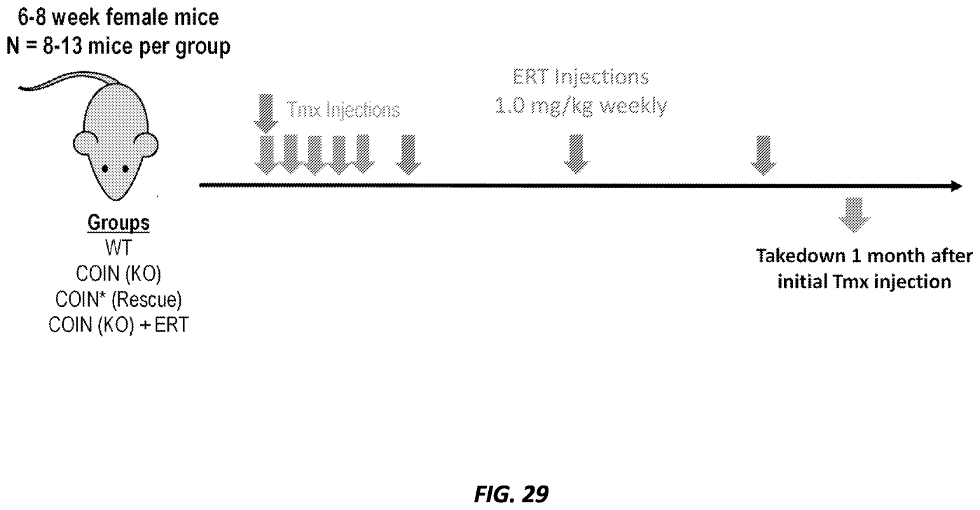

In order to discern to what degree the current therapeutic standard of care addresses MPS VI phenotypes, we next compared tamoxifen-induced rescue of Arsb expression in the Arsb COIN mice with enzyme replacement therapy (ERT) in which recombinant arylsulfatase B protein (also known as N-acetylgalactosamine-4-sulfatase or galsulfase) is administered. The experimental design is shown in FIG. 29. Four groups of mice (6-8 weeks old) were included: wild type mice, COIN (KO) mice, COIN* (rescue) mice treated with tamoxifen, and COIN (KO)+ERT. The COIN* (rescue) mice were treated with 5 daily intraperitoneal tamoxifen injections as described above. The COIN (KO)+ERT mice were treated with weekly retroorbital intravenous injections of 1 mg/kg recombinant arylsulfatase B protein. In addition, 50 mg/kg of an anti-CD4 blocking antibody was given one day prior to each injection to minimize immune responses to the ERT. The mice were taken down 1 month after the initial injections. WT and untreated COIN mice were used as additional control groups. All mice were μCT scanned at 26 days and euthanized 28 days post tamoxifen injection.

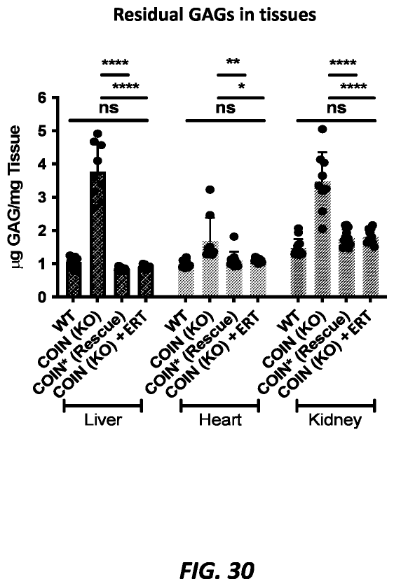

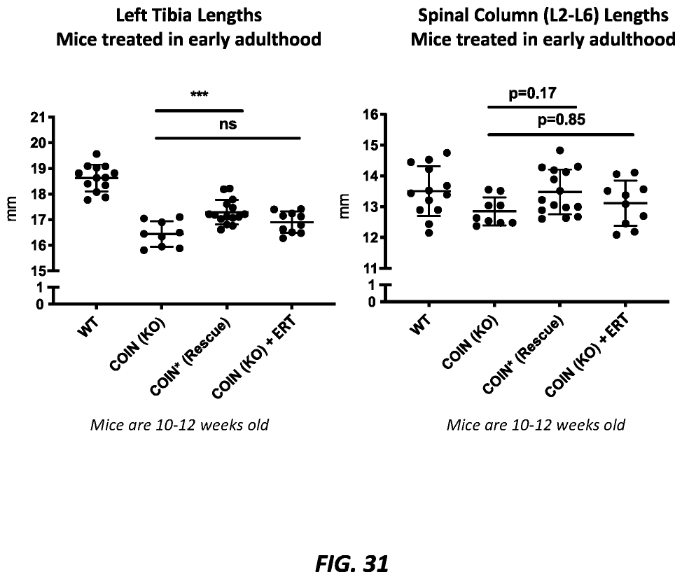

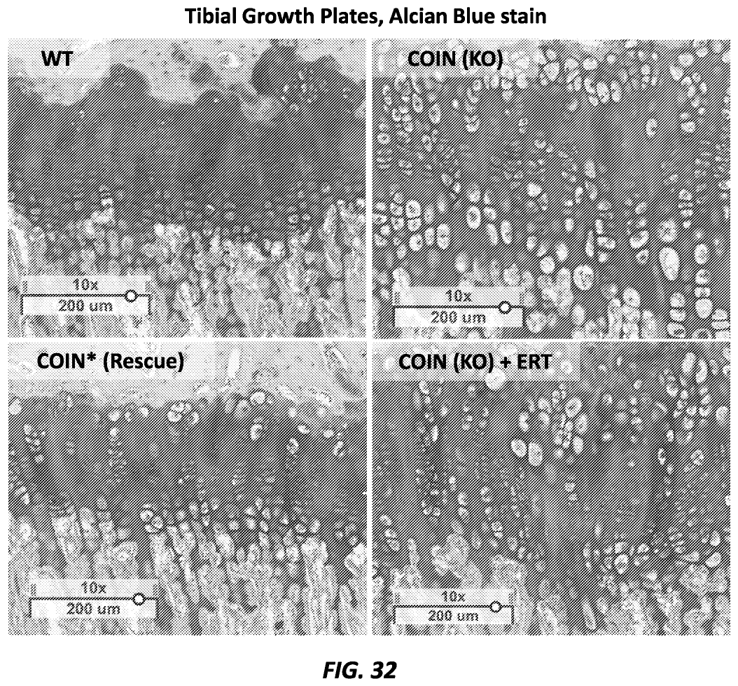

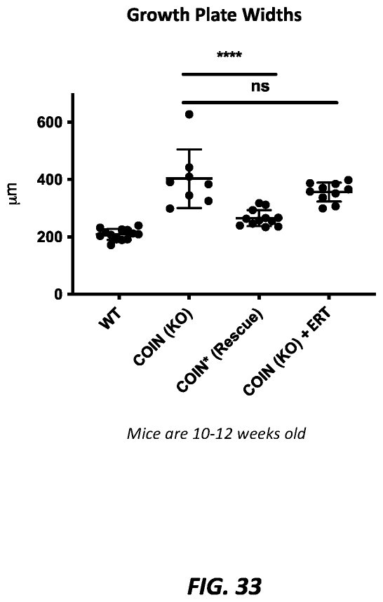

As shown in FIG. 30, both ERT and restoration of Arsb expression reduced residual GAGs in liver, heart, and kidney, while the untreated COIN (KO) control shows significantly elevated levels. However, as shown in FIG. 31, left tibia lengths were not rescued with 1 month of treatment with ERT at 1.0 mg/kg, whereas restoration of Arsb expression showed significant improvement. Similarly, tibial growth plate widths were improved (i.e. reduced) with Arsb restoration but not with ERT. See FIG. 32 and FIG. 33. In summary, Arsb COIN mice showed a Arsb knockout phenotype in the absence of Cre-mediated recombination. Tamoxifen treatment of Arsb COIN mice at age P21 or later in adulthood had minimal effect on skeletal length. In contrast, tamoxifen treatment of Arsb COIN mice at age P7 rescued skeletal length. Finally, comparison of Arsb restoration versus enzyme replacement therapy suggested that better treatment options may be able to improve skeletal outcomes in patients if treatment begins early.

Here, we present a new mouse model of MPS VI based on conditional-by-inversion (COIN) methodology, wherein the Arsb gene starts as null and can be restored using Cre. We observe elevated GAG storage in liver, heart, and kidney, along with multiple phenotypes in the skeletal system. Dysostosis multiplex is modeled in our mice, observed as shortened tibial length, spinal cord compression, and decreased cranial length to width ratios. We also observe tibial growth plate widening, along with markedly increased vacuolation. A major advantage of our approach is that we can model the effect of an optimal therapeutic by turning the Arsb gene back to WT, where it is transcriptionally regulated the same way as an original WT allele. In addition, our design allows for genetic controls, e.g., presence or absence of the Cre recombinase, and presence or absence of the floxed exon 5 inversion. This minimizes drug-specific effects as a confounder in phenotype interpretation since we can dose all groups.

For most tissue types examined and across ages, we see no significant fitness advantage of cell-intrinsic restoration of the Arsb allele, as recombination rates remain fairly consistent across the 1 month and 3 month timepoints.

To our knowledge, the COIN Arsb model described herein is the first mouse model in MPS VI research to show correction of the skeletal phenotype. Crucially, we show that with sufficiently early treatment, restoration of long bone length is possible. Skeletal readouts that become more pronounced with age—e.g., spinal column lengths and cranial length to width ratios—were also restored, although the magnitude of the effect in younger populations was less. Our most striking data are from mice treated at P7, in which we see a rescue to WT levels of tibial lengths as early as 1 month post tamoxifen treatment, along with rescue of spinal cord compression, cranial length to width ratios, and tibial growth plate widths. In mice treated with tamoxifen at P21, partial rescue is possible, while mice treated at P56-P70 are past the window for major bone growth, and exhibit little improvement. We have extended initial skeletal observations by six months with mice treated at P56-P70—these results confirm long bone length correction plateaus beyond 3 months post treatment (data not shown).

While most skeletal disease parameters seem to be abrogated to WT levels, increased vacuolation in the growth plate is not completely rescued at any age. It is unclear what the allele conversion rate is in the growth plate. The vacuolation phenotype may also be due to relative lack of metabolic/transcriptional activity of certain cell types, for which Arsb restoration may have little impact.

Taken together, these data imply that skeletal phenotypes are indeed treatable, and that exogenous enzyme may be limited in efficacy because of the poorly vascularized nature of the growth plate, and the ability of excess GAG to coordinate water. These data also imply that, when delivered early enough, an optimized therapeutic should be able to treat both stature and dysmorphia-related symptoms of MPS VI. Extrapolation of these data to humans based on estimates between human and mouse suggest rescue should be possible if treatment occurs within the first few years of life.

Our COIN model is remarkably well adapted to the study of lysosomal diseases, as it allows for stringent and complete loss of Arsb expression and function. The majority of lysosomal diseases are at least partly, if not completely, rescued by small amounts of residual enzymatic activity and expression, which is further compounded by secretion and uptake. Here, we show that this model can be used to define potential therapeutic parameters for refractory phenotypes incompletely corrected by current therapeutics.

### Materials and Methods

Recombination Assays. Genomic DNA were extracted from liver, heart, kidney, and spleen tissue harvested from MPS VI COIN and control mice using the GenElute Mammalian Genomic DNA Miniprep Kit (Sigma-Aldrich, G1N350-1KT). Genomic DNA from untreated COIN and WT mice were mixed at serial percentages to produce standard curves (100, 90, 75, 60, 40, 20, 0 percent COIN genomic DNA) on each plate. 50-100 ng of total genomic DNA were used per reaction in quantitative PCR using the 2× PowerUp SYBR Green Master Mix (ThermoFisher, A25742) with 1.0 μM of the following primers as appropriate: EHW092 (Arsb COIN fwd): AGGCCAAGATTGACAGTTACCAG (SEQ ID NO: 19); EHW093 (Arsb COIN rev): GGAGTACAGGGAAGGAAACCT (SEQ ID NO: 21); EHW094 (GAPDH fwd): CATGGCCTTCCGTGTTCCTA (SEQ ID NO: 51); EHW095 (GAPDH rev): CCTGCTTCACCACCTTCTTGAT (SEQ ID NO: 52). Technical duplicates for GAPDH reactions and technical triplicates for Arsb COIN reactions were used to assess average CT values. ACT values were compared to standards to assess percentage recombination.

RT-qPCR. For RNA Extraction: Tissue samples were homogenized in TRIzol, and chloroform was used for phase separation. The aqueous phase, containing total RNA, was purified using MagMAX™-96 for Microarrays Total RNA Isolation Kit (Ambion by Life Technologies) according to manufacturer's specifications. Genomic DNA was removed using RNase-Free Dnase Set (Qiagen).

For First Strand Synthesis and Quantitative PCR: mRNA was reverse-transcribed into cDNA using SuperScript® VILO™ Master Mix (Invitrogen by Life Technologies). cDNA was amplified with the SensiFAST Probe Lo-ROX (Meridian) using the 12K Flex System (Applied Biosystems). GAPDH was used to normalize any cDNA input differences. Primer sequences used were the following: Arsb fwd—CCAAACCTCTGGATGGCTTCAAC (SEQ ID NO: 53); Arsb rev—GTCCTGATCGATGTTGTGTAGCAG (SEQ ID NO: 54); Arsb probe—AAGACAATCAGTGAAGGACACCCATCCC (SEQ ID NO: 55); Gapdh fwd—TGCCCAGAACATCATCCCT (SEQ ID NO: 56); Gapdh rev—GGAGGCCATGCCAGTGAG (SEQ ID NO: 57); Gapdh probe—ATCCACTGGTGCTGCCAAGGCTG (SEQ ID NO: 58). Data were acquired in technical triplicate and were calculated with mean CT using the ΔΔCT method, with WT samples as references.

GAG Accumulation Assays. Tissue Digestion for Glycosaminoglycan Isolation Finely minced organ tissue was resuspended at a concentration of 10 mg/mL in 0.3 mg/mL papain+2 mM DTT and incubated at 60° C. for 1.5 hours, with samples gently vortexed every 15 minutes. 10 μL of 1M acetic acid and 40 μL of Tris-HCl (pH 8.0) were added per 1 mL of organ lysate and mixed well (AMSBio, 280560-TDK).

Sulfated Glycosaminoglycan Quantitation. A standard curve was produced using five 4-fold serial dilutions from 100 μg/mL chondroitin sulfate. 4-fold dilutions of each sample was created in duplicate in 1× assay buffer (AMSBio, 280560-N). 100 μL of standards and experimental samples were transferred to a clear bottom plate, then mixed with 100 μL of 100 mM dimethylmethylene blue. Absorbances at 515 nm were assessed within 15 minutes and compared to standard curves (AMSBio, 280560-N).

Skeletal Analyses. For μCT analyses, mice were anesthetized using isoflurane and dynamically scanned using 17s×2 or 8s×3 algorithms on Quantum FX or Quantum GX computerized tomographs (PerkinElmer) to produce whole skeletal images. Stitched vox files were analyzed using Analyze14.0 software. All lengths were determined using the line draw tool and double checked with single-blinded analyses. For high resolution μCT femoral analyses, mice were C02-euthanized and left femurs were extracted then drop fixed in 10% formalin (VWR, 16004-121). Samples were incubated rotating at 4° C. for 48 hours, washed 4 times with 1×DPBS (Gibco, 14040), and stored in 70% ethanol until scans on a Scanco μCT35. Transverse CT slices were evaluated in the region starting 360 μm proximal to the growth plate and extending 1440 μm. The trabecular bone region was identified manually by tracing the region of interest. Images were threshholded using an adaptive-iterative algorithm and morphometric variables were computed from the binarized images using direct, 3D techniques that do not rely on any prior assumptions about the underlying structure. Transverse CT slices were also evaluated in the midshaft region, extending 120 μm.

Histology and Growth Plate Analyses. Right knee joints were harvested from freshly CO₂-euthanized mice and drop fixed in 4% paraformaldehyde. Samples were fixed for 48 hours, decalcified, embedded in paraffin, and sectioned at two levels on the longitudinal axis. Sections were stained with Alcian blue with fast red counterstain, or Safranin O with fast green counterstain (Histoserv). Growth plate widths were assessed on Alcian Blue stained samples as a mean with HALO software (Indica Labs), using the layer thickness tool at an approximate interval of 55 μM with maximal smoothing. Vacuolation was assessed on SafO stained samples with HALO software using the Vacuole Module as total vacuolated area over total growth plate area.

Mouse Lines and Injections. Mouse Line Generation. A large targeting vector (LTVEC) with a 5′ homology arm comprising 81 kb of the mouse Arsb locus and 3′ homology arm comprising 102 kb of the mouse Arsb locus was generated to replace a region surrounding exon 5 with a corresponding inverted region flanked by a 5′ lox71 site and a 3′ lox66. A roxed self-deleting cassette was inserted downstream of the inverted sequence. To generate the mutant allele, the LTVEC was introduced into mouse embryonic stem cells. Specifically, 2×10⁶ mouse VGF1 embryonic cells (50% C57BL/6NTac 50% 12956/SvEvTac) carrying CreER^{T2} in the Gt(ROSA26)Sor locus were electroporated with 0.4 μg mArsb LTVEC. Antibiotic selection was performed using G418 at a concentration of 100 mg/mL. Colonies were picked, expanded, and screened by TAQMAN®. F0 mice were generated from above modified ES cells using the VELOCIMOUSE® method. Specifically, mouse ES cell clones described above were selected and injected into 8-cell stage embryos using the VELOCIMOUSE® method.

Injections. For P7, P21, and P56-P70 restoration experiments, tamoxifen (Sigma, T5648) was resuspended in sterile corn oil (Sigma, C8267) at 10 mg/mL or 5 mg/mL and injected via the intraperitoneal route at the appropriate doses normalized to mouse weight daily for five consecutive days. T cell depletion was accomplished with intraperitoneal injection of an α-CD4 antibody (BioXCell, BE0003-3) at 50 mg/kg, weekly. Galsulfase delivery was accomplished with retro-orbital intravenous injection of recombinant human ARSB (BioMarin, Naglazyme lot L061938) at 1 mg/kg, weekly. Antibodies and enzymes were diluted to appropriate doses in 0.9% sodium chloride (Intermountain Life Sciences, Z1376).

Flow Cytometry. Freshly harvested spleens were placed into cold RPMI (Irvine Scientific, 9161), manually dissociated and pushed through a 74 μM filter (Costar, 3477). Splenocytes were washed once with cold RPMI, and RBC lysis buffer was added (Sigma, R7757). After a 2-minute incubation, cells were washed twice with cold DPBS (Gibco, 14040), filtered again through a 100 μM filter (EMD Millipore, MANM10010), and resuspended in 1 mL of cold DPBS. 100 μL of each sample was used for experimental staining, and 100 μL of each sample was pooled for use in FMOs and controls. Cells were stained with a 500× dilution of Live/Dead Blue (Invitrogen, L34962)×15 minutes, washed, then blocked with CD16/32 (Tonbo, 70-0161-M001) diluted 50× in MACS Buffer (Miltenyi Biotec, 130-091-221)×15 minutes at 4° C. Appropriate antibodies were added at the below dilutions resuspended in Brilliant Stain Buffer (Becton Dickinson, 566349) and incubated at 4° C.×30 minutes. Splenocytes were washed twice with cold MACS Buffer and fixed with Cytofix (Becton Dickinson, 554655) at 4° C.×15 minutes. Splenocytes were washed twice with cold DPBS then resuspended in MACS Buffer. Cells were filtered a final time (Pall, 8027) before acquisition on a LSRFortessa X-20 flow cytometer (Becton Dickinson). Population percentages and scatter profiles were assessed using FlowJo, all gates were drawn against FMO samples, and data were plotted using Prism.

| Name/Antigen | Conjugate | Reactivity | Host | Clone | Isotype | Supplier | Cat # | Dilution (1/x) |
|---|---|---|---|---|---|---|---|---|
| CD8 | PerCP-Cy5.5 | Mouse | Rat | 53-6.7 | IgG2a, K | Biolegend | 100734 | 200 |
| XCR1 | BV421 | Mouse/Rat | Mouse | ZET | IgG2b, k | Biolegend | [symbol] 148216 | 200 |
| CD19 | BV510 | Mouse | Rat | 6D5 | IgG2a, K | Biolegend | [symbol] 115545 | 200 |
| CD11b | BV605 | Mouse/Human | Rat | M1/70 | IgG2b, K | BD | [symbol] 563015 | 200 |
| F4/80 | BV711 | Mouse | Rat | T45-2342 | IgG2a, K | BD | [symbol] 565612 | 50 |
| CD3 | BV786 | Mouse | Hamster | 145-2C11 | IgG1, K | BD | [symbol] 564379 | 200 |
| B220 (aka | BUV395 | Mouse | Rat | RA3-6B2 | IgG2a, K | BD | [symbol] 563793 | 200 |
| CD45R) | | | | | | | | |
| CD11c | BUV737 | Mouse | Hamster | HL3 | IgG1, A2 | BD | [symbol] 564986 | 200 |
| CD4 | BUV805 | Mouse | Rat | GK1.5 | IgG2b, k | BD | [symbol] 564922 | 200 |
| Ly6G | APC | Mouse | Rat | 1A8-Ly6g | IgG2a, K | Invitrogen/ | 17-9668-82 | 200 |
| | | | | | | TF/eBioscience | | |
| I-A/I-E | lexa Fluor 70 | Mouse | Rat | M5/114.15.2 | IgG2b, K | Biolegend | [symbol] 107622 | 200 |
| pDCA-1 | PE-Cy7 | Mouse | Rat | eBio927 | IgG2b, K | Invitrogen/TF/ | 25-3172-82 | 200 |
| (aka CD317) | | | | | | eBioscience | | |

### Example 3. Generation of a COIN Mouse Model of Mucopolysaccharidosis I

Mucopolysaccharidosis I (MPS I) is a lysosomal disease resulting from impaired function of the alpha-L-iduronidase (IDUA) protein. To date, no models of disease exist that separate treatment efficacy from disease reversibility.

To this end, we have generated a “conditional-on” mouse model of MPS I, using a Rosa Cre-ERT2 plus floxed-inversion approach (COIN). This approach allowed us to turn back “on” endogenous expression of the missing enzyme at several points during disease progression.

A large targeting vector (LTVEC) comprising a 5′ homology arm comprising 46 kb of the mouse Idua locus and 3′ homology arm comprising 38 kb of the mouse Idua locus was generated to replace a region from intron 3 through the 3′ UTR with a corresponding inverted region flanked by a 5′ lox71 site and a 3′ lox66. A roxed self-deleting cassette was inserted downstream of the inverted gene. Information on the mouse Idua gene is provided in Table 8. A description of the generation of the large targeting vector is provided in Table 9. Generation and use of large targeting vectors (LTVECs) derived from bacterial artificial chromosome (BAC) DNA through bacterial homologous recombination (BHR) reactions using VELOCIGENE® genetic engineering technology is described, e.g., in U.S. Pat. No. 6,586,251 and Valenzuela et al. (2003) Nat. Biotechnol. 21(6):652-659, each of which is herein incorporated by reference in its entirety for all purposes. Generation of LTVECs through in vitro assembly methods is described, e.g., in US 2015/0376628 and WO 2015/200334, each of which is herein incorporated by reference in its entirety for all purposes.

TABLE 8
Mouse Idua.

| | Gene | NCBI | RefSeq | UniProt | | |
|---|---|---|---|---|---|---|
| | Symbol | Gene ID | mRNA ID | ID | Genomic Assembly | Chromosomal Location |
| Mouse | Idua | 15932 | NM_008325.4 | P48441 | GRCm38/mm10 | chr5:108,660,331- |
| | | | | | | 108,684,552 (+) |

TABLE 9
Mouse Idua Large Targeting Vector.

| | | | | Length |
|---|---|---|---|---|
| | Genome Build | Start | End | (bp) |
| 5′ Mouse Arm | GRCm38/mm10 | chr5: | chr5: | 46,205 |
| | | 108,632,847 | 108,679,152 | |
| Human Insert | GRCm38/mm10 | chr5: | chr5: | 5,810 |
| | | 108,684,982 | 108,679,173 | |
| 3′ Mouse Arm | GRCm38/mm10 | chr5: | chr5: | 38,286 |
| | | 108,685,003 | 108,723,289 | |

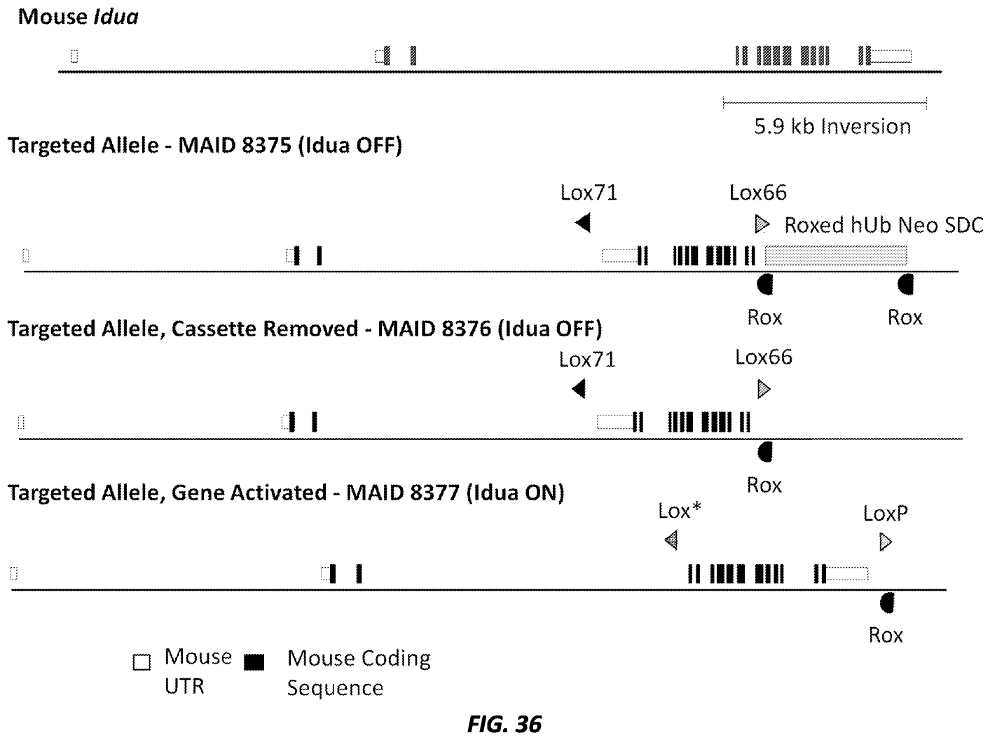

Specifically, a region from intron 3 through the 3′ UTR of the Idua gene was inverted in the mouse Idua locus. The inverted region (SEQ ID NO: 49) is flanked by a 5′ lox71 site and a 3′ lox66. A roxed self-deleting cassette was inserted 3′ of the inverted region. This is the MAID 8375 allele (SEQ ID NO: 10). The allele after cassette deletion is the MAID 8376 allele (SEQ ID NO: 11). The cassette-deleted allele after rescue by inversion of the inverted region (reinverted region set forth in SEQ ID NO: 50) is the MAID 8377 allele (SEQ ID NO: 12). See FIG. 36.

The expected encoded IDUA protein after rescue by inversion of the inverted region is a fully mouse IDUA protein. See FIG. 36. The mouse IDUA protein and Idua coding sequences are set forth in SEQ ID NOS: 4 and 6, respectively.

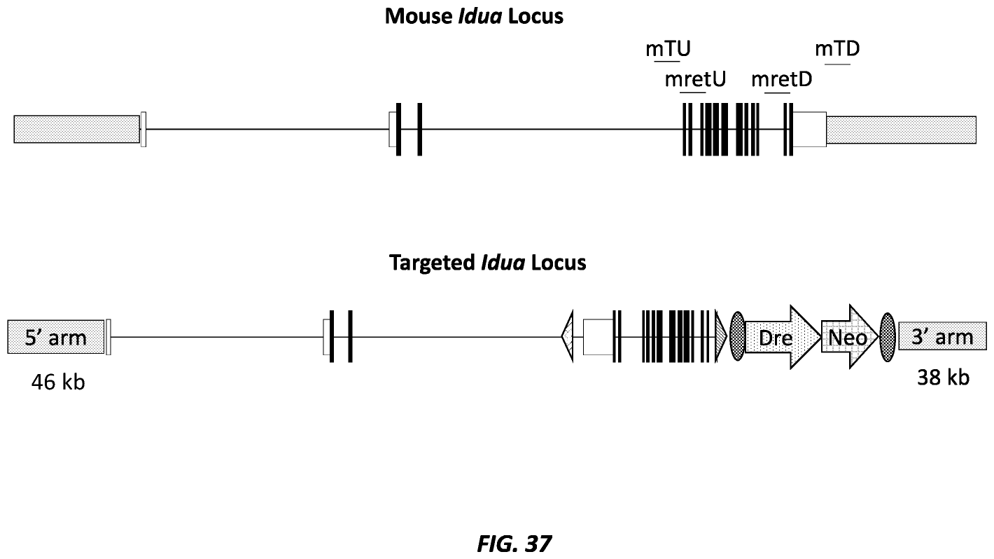

To generate the mutant allele a large targeting vector was introduced into mouse embryonic stem cells. Specifically, 2×10⁶ mouse VGF1 embryonic cells (50% C57BL/6NTac 50% 129S6/SvEvTac) carrying a Rosa26-CreER^{T2} allele were electroporated with 0.4 μg mIdua COIN LTVEC. The electroporation conditions were: 400 V voltage; 100 mF capacitance; and 0 W resistance. Antibiotic selection was performed using G418 at a concentration of 100 mg/mL. Colonies were picked, expanded, and screened by TAQMAN®. See FIG. 37. Loss-of-allele assays were performed to detect loss of the endogenous mouse allele, and retention assays were performed using the primers and probes set forth in Table 10. To detect the mIdua gene inversion, after tamoxifen-Cre activation, mouse tissue was assayed by qPCR using the primers and probes set forth in Table 10.

TABLE 10
Screening Assays.

| | | | | SEQ |
|---|---|---|---|---|
| | | | | ID |
| Assay | Description | Primer/Probe | Sequence | NO |
| mTU | Upstream LOA | Fwd | TCACGTCTTTCCACCTCAGTG | 31 |
| | | Probe (FAM) | TGACTGACCAGAGCCCTACTTGA | 32 |
| | | Rev | GTGTGTGTCTGAAACCTTCAAGA | 33 |
| mTD | Downstream LOA | Fwd | GGCTGATGTTCCTACCTTGGTT | 34 |
| | | Probe (Cal-Orange) | CTGGCCACGGGAAGAACCCTCT | 35 |
| | | Rev | TGTGTCCAACAGGCTCTCT | 36 |
| mretU | Upstream | Fwd | GGGCAGGGACTTATGTACAAC | 37 |
| | Retention Assay | Probe (FAM) | TCACCCACTTGGATGCATTCTTGGA | 38 |
| | | Rev | GCTCACCAGGGAGAAGCT | 39 |
| mretD | Downstream | Fwd | TCCCAGAAAGGTGAAGAGTATGC | 40 |
| | Retention Assay | Probe (Cal) | CCCAATCAACAGGAGGCCGTCTAC | 41 |
| | | Rev | GGGCTGAACACAAAGAGGTTA | 42 |
| 8377JXmU | 5′ Junction mIdua- | Fwd | GTCACCTGTCTCACGTCTTTC | 43 |
| | ON | Rev | GTGAATGTGTGTGTCTGAAACC | 44 |
| 8377JXmD | 3′ Junction mIdua- | Fwd | AGCCTGGGCTGATCTTAATAG | 45 |
| | ON | Rev | CGCTACCTTAGGACCGTTATAG | 46 |

Modification-of-allele (MOA) assays including loss-of-allele (LOA) and gain-of-allele (GOA) assays are described, for example, in US 2014/0178879; US 2016/0145646; WO 2016/081923; and Frendewey et al. (2010) Methods Enzymol. 476:295-307, each of which is herein incorporated by reference in its entirety for all purposes. The loss-of-allele (LOA) assay inverts the conventional screening logic and quantifies the number of copies in a genomic DNA sample of the native locus to which the mutation was directed. In a correctly targeted heterozygous cell clone, the LOA assay detects one of the two native alleles (for genes not on the X or Y chromosome), the other allele being disrupted by the targeted modification. The same principle can be applied in reverse as a gain-of-allele (GOA) assay to quantify the copy number of the inserted targeting vector in a genomic DNA sample.

Retention assays are described in US 2016/0145646 and WO 2016/081923, each of which is herein incorporated by reference in its entirety for all purposes. Retention assays distinguish between correct targeted insertions of a nucleic acid insert into a target genomic locus from random transgenic insertions of the nucleic acid insert into genomic locations outside of the target genomic locus by assessing copy numbers of DNA templates from 5′ and 3′ target sequences corresponding to the 5′ and 3′ homology arms of the targeting vector, respectively. Specifically, retention assays determine copy numbers in a genomic DNA sample of a 5′ target sequence DNA template intended to be retained in the modified target genomic locus and/or the 3′ target sequence DNA template intended to be retained in the modified target genomic locus. In diploid cells, correctly targeted clones will retain a copy number of two. Copy numbers greater than two generally indicate transgenic integration of the targeting vector randomly outside of the target genomic locus rather than at the target genomic locus. Copy numbers of less than generally indicate large deletions extending beyond the region targeted for deletion.

F0 mice were generated from the modified ES cells using the VELOCIMOUSE® method. Specifically, mouse ES cell clones comprising the modified Idua locus described above that were selected by the MOA assay described above were injected into 8-cell stage embryos using the VELOCIMOUSE® method. See, e.g., U.S. Pat. Nos. 7,576,259; 7,659,442; 7,294,754; US 2008/0078000; and Poueymirou et al. (2007) Nat. Biotechnol. 25(1):91-99, each of which is herein incorporated by reference in its entirety for all purposes. In the VELOCIMOUSE® method, targeted mouse ES cells are injected through laser-assisted injection into pre-morula stage embryos, e.g., eight-cell-stage embryos, which efficiently yields F0 generation mice that are fully ES-cell-derived. In the VELOCIMOUSE® method, the injected pre-morula stage embryos are cultured to the blastocyst stage, and the blastocyst-stage embryos are introduced into and gestated in surrogate mothers to produce the F0 generation mice. When starting with mouse ES cell clones homozygous for the targeted modification, F0 mice homozygous for the targeted modification are produced. When starting with mouse ES cell clones heterozygous for the targeted modification, subsequent breeding can be performed to produce mice homozygous for the targeted modification.

### Example 4. Use of COIN Model in MPS I to Define Phenotype Reversibility

The mouse model generated in Example 3 allows restoration of Idua expression from the native locus at desired timepoints, bypassing the limitations of enzyme replacement therapy (ERT). In the absence of tamoxifen, IDUA production is “off,” replicating the Idua knockout phenotype, because the region from intron 3 through the 3′ UTR of the Idua gene is inverted. In the presence of tamoxifen, expression of Cre recombinase is induced, and the Cre recombinase inverts the inverted region, flipping it back into the correct orientation, restoring wild type Idua expression.

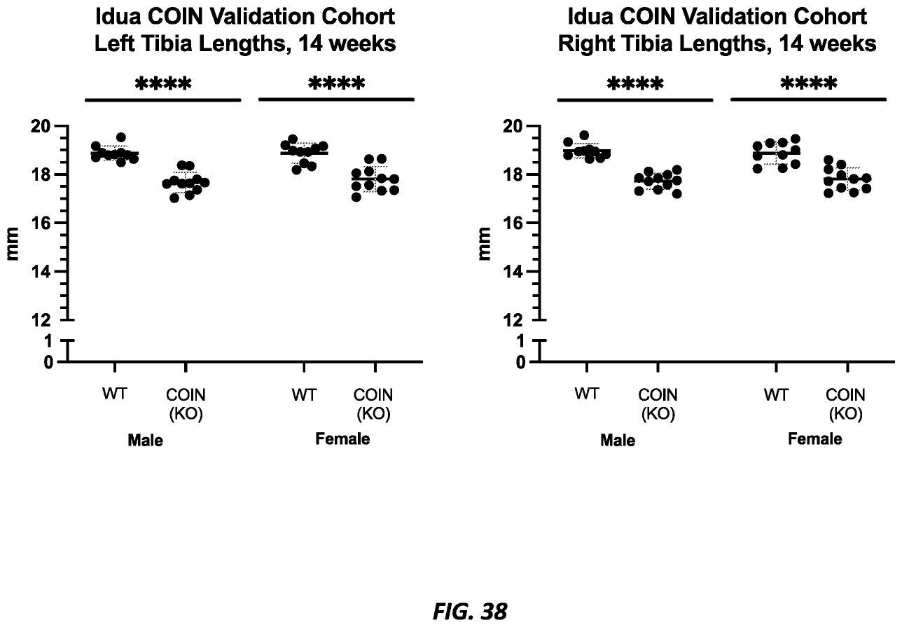

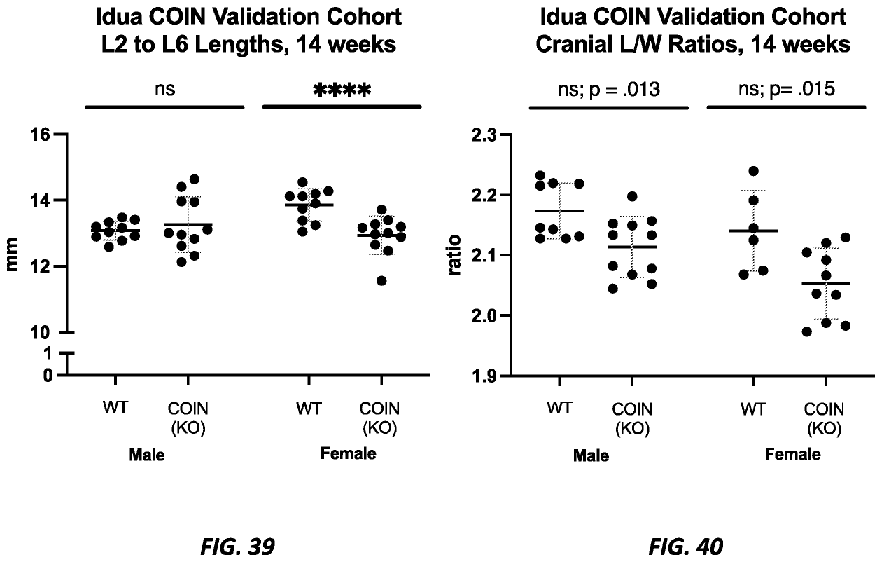

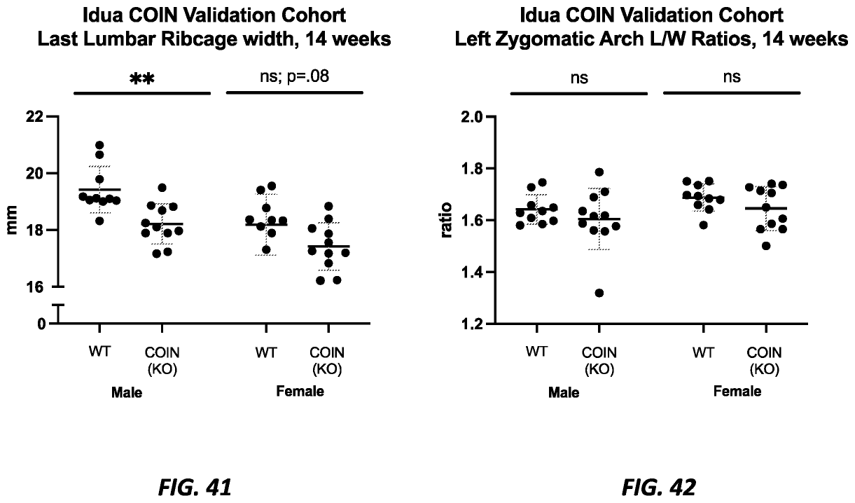

The COIN Idua mice were first assessed in the absence of tamoxifen to assess whether they display key features of MPS I. COIN Idua mice were examined alongside an age and sex matched wild type (WT) cohort for skeletal and peripheral tissue phenotypes. These mice were anesthetized prior to micro computerized tomography (μCT) scans. Two days later they were CO₂-euthanized and liver, heart, spinal cord, spleen, kidney, and sera were harvested. Total sulfated GAG in each tissue type was assessed using dimethylmethylene blue (DMMB), which is a chromogen that presents an absorption change upon binding the sulfated moiety in GAGs. The COIN Idua mice without tamoxifen treatment showed decreased tibia lengths, decreased spinal column lengths, decreased cranial length:width ratio, restricted ribcage width, and increased GAG accumulation in liver, heart, and kidneys compared to wild type mice but not significant difference in zygomatic arch L/W ratios. See FIGS. 38-42 and 44. Thus, the COIN Idua mice replicate an MPS I skeletal phenotype (shorter tibias, spinal column length, cranial length to width ratios, and ribcage widths) and replicate a GAG accumulation phenotype (in the liver, heart, and kidney).

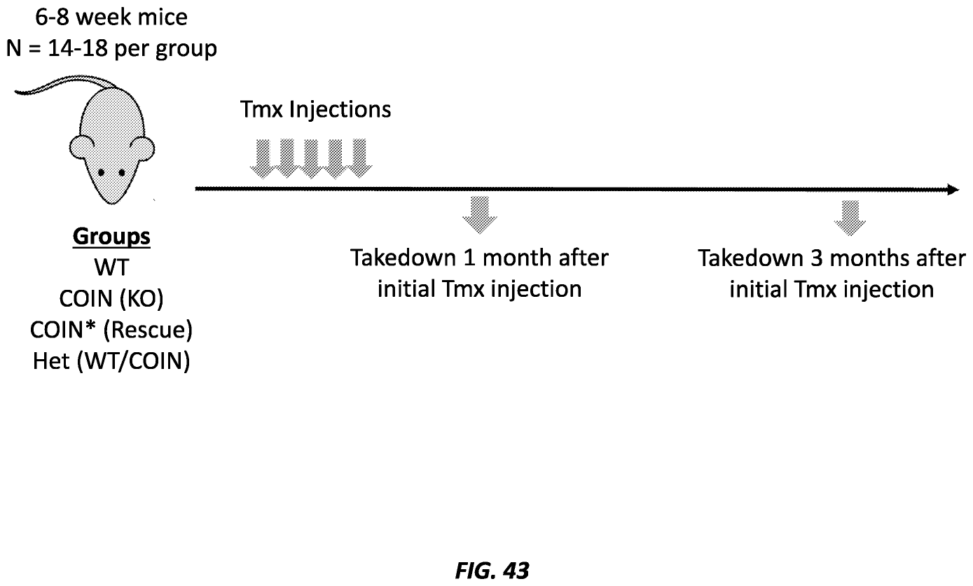

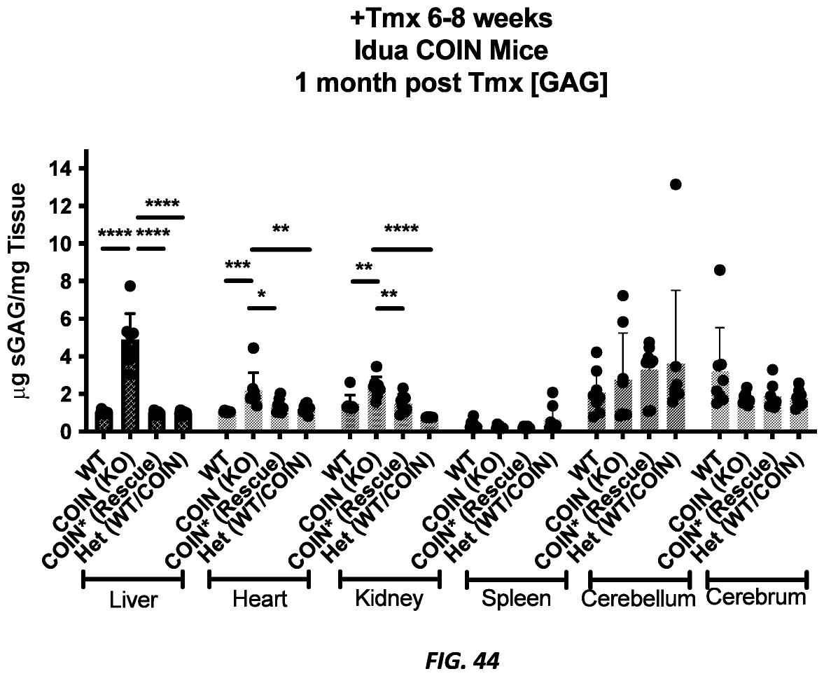

To assess phenotype reversibility at different timepoints, the COIN Idua mice described in Example 3 are treated with 5 daily tamoxifen injections starting at different timepoints, similar to Example 2. Mice are taken down at different time points after tamoxifen injection. Behavioral, histological, and molecular readouts are assessed at the different time points. In a first experiment, the COIN Idua mice described in Example 3 are treated with 5 daily tamoxifen injections starting at 8-10 weeks after birth, and mice were taken down at 1 month and 3 months after tamoxifen injection. See FIG. 43. As shown in FIG. 44, GAG accumulation occurs in liver, heart, and kidneys in the knockout mice, and this is rescued by one month post-tamoxifen treatment.

Behavioral testing includes open field, fear conditioning, and elevated plus maze experiments as described below. For COIN Idua mice (IDUA production is “off”) we would expect decreased exploratory behavior on the open field, decreased anxiety on the elevated plus (more time spent in open arms), and impaired long term memory in fear conditioning.

Open Field. Kinder Scientific apparatus is used. Mice are placed in the open field and allowed to move freely while they are monitored for 15 to 120 minutes. The apparatus uses infrared beams and computer software to calculate fine movements, X+Y ambulation, distance traveled, number of rearing events, time spent rearing, and immobility time.

Fear Conditioning. A shuttlebox apparatus is used that is equipped with a tone generator and a gridded floor through which shock can be delivered. Mice are acclimated to the procedure room for 45 minutes to 1 hour prior to beginning test. For training on day 1, animals are taught to associate a tone and/or the context with a footshock. Footshock is paired with a tone by sequentially or simultaneously administering a shock with the tone (no more than 5 seconds, 0.8 mA). Footshock is paired with the context by shocking the animal once it is placed in the box. Some amount of conditioning to context is also apparent from the tone-shock animals. Learning can occur with as little as one shock. Animals are never shocked more than 10 times to achieve this learning. On day 2, learning is evaluated by later measuring the animal's freezing behavior in response to the tone or the context (the box in which the animal was shocked). Controls for non-specific freezing are exposure to a novel context which has never been associated with a shock and exposure to a tone which was either never paired with the shock, or was present with the shock, but in a non-predictive, non-associative fashion. Because mice do not freeze for long periods of time, they are evaluated for freezing behavior in 5 second time bins. They receive a score of 0 for every bin in which they move, and a score of 1 for every bin in which they do not. Mice are evaluated for 30 seconds to 5 minutes. Mice with higher scores are considered more fearful, and will hence have learned the association better.

Elevated Plus Maze. The elevated plus maze is a widely used behavioral task for rodents to measure anxiety. The basic measure of this task is the animals' preference for dark and not exposed places (Walf and Frye (2007) Nat. Protoc. 2(2):322-328, herein incorporated by reference in its entirety for all purposes). The maze consists of two open and two closed arms. The arms form a cross shape with the two similar arms opposite to each other. The animals are placed at the center of the maze, facing an enclosed arm. The behavior of the animals is recorded for 5 minutes with a camera located above the maze. Time spent in the different compartments (closed and open arms; also center) is measured. The maze is cleaned with 70% ethanol between trials. Anxiety is assessed by comparing the activity in the open versus closed arms, using an index taking into account the time spent in each category of arm as the ratio open arms/closed arms. The relationship between index value and anxiety level is inversely analogous, meaning that the higher the index value the lower the level of anxiety.

We claim:

1. A non-human animal comprising in its genome a reverse-conditional null endogenous lysosomal storage disease gene, wherein the reverse-conditional null endogenous lysosomal storage disease gene is a null endogenous lysosomal storage disease gene that is converted to a functional endogenous lysosomal storage disease gene upon treatment with a recombinase.

2. The non-human animal of claim 1, wherein a critical region of the endogenous lysosomal storage disease gene is inverted and flanked by recombinase recognition sites in the reverse-conditional null endogenous lysosomal storage disease gene, wherein the recombinase recognition sites are oriented such that the critical region is reinverted upon treatment with the recombinase.

3. The non-human animal of claim 2, wherein the critical region comprises a coding sequence of the endogenous lysosomal storage disease gene.

4. The non-human animal of claim 2 or 3, wherein the recombinase is a Cre recombinase.

5. The non-human animal of claim 4, wherein the recombinase recognition sites comprise a lox71 site and a lox66 site.

6. The non-human animal of any one of claims 2-5, wherein the non-human animal further comprises a genomically integrated recombinase expression cassette encoding the recombinase.

7. The non-human animal of claim 6, wherein the genomically integrated recombinase expression cassette comprises a recombinase coding sequence operably linked to a tissue-specific promoter.

8. The non-human animal of claim 6 or 7, wherein the recombinase is inducible.

9. The non-human animal of claim 8, wherein the recombinase is inducible upon treatment with tamoxifen.

10. The non-human animal of any preceding claim, wherein the reverse-conditional null endogenous lysosomal storage disease gene comprises a selection cassette or a reporter gene flanked by recombinase recognition sites for a second recombinase.

11. The non-human animal of claim 10, wherein the reverse-conditional null endogenous lysosomal storage disease gene comprises the selection cassette, wherein the selection cassette is a self-deleting selection cassette.

12. The non-human animal of any one of claims 1-9, wherein the non-human animal does not comprise a selection cassette or a reporter gene.

13. The non-human animal of any preceding claim, wherein the non-human animal comprises the reverse-conditional null endogenous lysosomal storage disease gene in its germline.

14. The non-human animal of any preceding claim, wherein the non-human animal is a mammal.

15. The non-human animal of any preceding claim, wherein the non-human animal is a rodent.

16. The non-human animal of any preceding claim, wherein the non-human animal is a mouse or a rat.

17. The non-human animal of claim 16, wherein the non-human animal is the mouse.

18. The non-human animal of any preceding claim, wherein the lysosomal storage disease gene is a mucopolysaccharidosis gene.

19. The method of any preceding claim, wherein the lysosomal storage disease gene is an Arsb gene.

20. The non-human animal of claim 19, wherein a critical region of the endogenous Arsb gene is inverted and flanked by recombinase recognition sites in the reverse-conditional null endogenous Arsb gene, wherein the recombinase recognition sites are oriented such that the critical region is reinverted upon treatment with the recombinase.

21. The non-human animal of claim 20, wherein the critical region comprises exon 5 of the endogenous Arsb gene.

22. The non-human animal of any one of claims 19-21, wherein the non-human animal displays one or more phenotypes associated with mucopolysaccharidosis VI.

23. The non-human animal of claim 22, wherein the non-human animal displays one or more skeletal phenotypes associated with mucopolysaccharidosis VI.

24. The non-human animal of any one of claims 19-23, wherein the non-human animal displays one or more or all of the following relative to a wild type non-human animal:
(a) increased accumulation of glycosaminoglycans in the liver;
(b) increased accumulation of glycosaminoglycans in the heart;
(c) increased accumulation of glycosaminoglycans in the kidney;
(d) decreased tibia length;
(e) decreased spinal column length;
(f) increased tibial growth plate width; and
(g) decreased cranial length:width ratio.

25. The non-human animal of any one of claims 19-24, wherein the non-human animal displays all of the following relative to the wild type non-human animal:
(a) increased accumulation of glycosaminoglycans in the liver;
(b) increased accumulation of glycosaminoglycans in the heart;
(c) increased accumulation of glycosaminoglycans in the kidney;
(d) decreased tibia length;
(e) decreased spinal column length;
(f) increased tibial growth plate width; and
(g) decreased cranial length:width ratio.

26. The non-human animal of claim 19, wherein a region comprising exon 5 of the endogenous Arsb gene is inverted and flanked by recombinase recognition sites in the reverse-conditional null endogenous Arsb gene,
wherein the recombinase recognition sites are oriented such that the region is reinverted upon treatment with the recombinase,
wherein the recombinase is a Cre recombinase, and the recombinase recognition sites comprise a lox71 site and a lox66 site,
wherein the non-human animal comprises the reverse-conditional null endogenous Arsb gene in its germline,
wherein the non-human animal is a mouse, and
wherein the non-human animal displays one or more phenotypes associated with mucopolysaccharidosis VI.

27. The non-human animal of claim 26, wherein the one or more phenotypes comprise one or more or all of the following relative to a wild type non-human animal:
(a) increased accumulation of glycosaminoglycans in the liver;
(b) increased accumulation of glycosaminoglycans in the heart;
(c) increased accumulation of glycosaminoglycans in the kidney;
(d) decreased tibia length;
(e) decreased spinal column length;
(f) increased tibial growth plate width; and
(g) decreased cranial length:width ratio.

28. The non-human animal of claim 27, wherein the non-human animal displays the decreased tibia length, wherein the decreased tibia length is rescuable within one month after treatment with the recombinase at post-natal day 7 or earlier but is not rescuable within one month after treatment with the recombinase at post-natal day 21 or later or at 8 weeks after birth or later.

29. The non-human animal of claim 27, wherein the non-human animal displays the decreased spinal column length, wherein the decreased spinal column length is rescuable within one month after treatment with the recombinase at post-natal day 7 or earlier but is not rescuable within one month after treatment with the recombinase at post-natal day 21 or later or at 8 weeks after birth or later.

30. The non-human animal of claim 27, wherein the non-human animal displays the increased tibial growth plate width, wherein the increased tibial growth plate width is rescuable within one month after treatment with the recombinase at post-natal day 7 or earlier but is not rescuable within one month after treatment with the recombinase at post-natal day 21 or later or at 8 weeks after birth or later.

31. The non-human animal of claim 27, wherein the non-human animal displays the decreased cranial length:width ratio, wherein the decreased cranial length:width ratio is rescuable within one month after treatment with the recombinase at post-natal day 7 or earlier but is not rescuable within one month after treatment with the recombinase at post-natal day 21 or later or at 8 weeks after birth or later.

32. The non-human animal of any one of claims 19-31, wherein the reverse-conditional null endogenous Arsb gene comprises the sequence set forth in SEQ ID NO: 7, 8, or 47.

33. The non-human animal of any one of claims 19-32, wherein upon treatment the reverse-conditional null endogenous Arsb gene comprises the sequence set forth in SEQ ID NO: 9 or 48.

34. The method of any one of claims 1-18, wherein the lysosomal storage disease gene is an Idua gene.

35. The non-human animal of claim 34, wherein a critical region of the endogenous Idua gene is inverted and flanked by recombinase recognition sites in the reverse-conditional null endogenous Idua gene, wherein the recombinase recognition sites are oriented such that the critical region is reinverted upon treatment with the recombinase.

36. The non-human animal of claim 35, wherein the critical region comprises a region from exon 2 to the stop codon of the endogenous Idua gene.

37. The non-human animal of any one of claims 34-36, wherein the non-human animal displays one or more phenotypes associated with mucopolysaccharidosis I.

38. The non-human animal of claim 37, wherein the non-human animal displays one or more skeletal phenotypes associated with mucopolysaccharidosis I.

39. The non-human animal of claim 37, wherein the non-human animal displays one or more neurological phenotypes associated with mucopolysaccharidosis I.

40. The non-human animal of claim 37, wherein the non-human animal displays one or more skeletal phenotypes associated with mucopolysaccharidosis I and one or more neurological phenotypes associated with mucopolysaccharidosis I.

41. The non-human animal of any one of claims 34-40, wherein the non-human animal displays one or more or all of the following relative to a wild type non-human animal:
(a) increased accumulation of glycosaminoglycans in the liver;
(b) increased accumulation of glycosaminoglycans in the heart;
(c) increased accumulation of glycosaminoglycans in the kidney;
(d) decreased tibia length;
(e) decreased spinal column length;
(f) decreased ribcage width; and
(g) decreased cranial length:width ratio.

42. The non-human animal of any one of claims 34-41, wherein the non-human animal displays all of the following relative to the wild type non-human animal:
(a) increased accumulation of glycosaminoglycans in the liver;
(b) increased accumulation of glycosaminoglycans in the heart;
(c) increased accumulation of glycosaminoglycans in the kidney;
(d) decreased tibia length;
(e) decreased spinal column length;
(f) decreased ribcage width; and
(g) decreased cranial length:width ratio.

43. The non-human animal of any one of claims 34-42, wherein the non-human animal displays one or more or all of the following relative to a wild type non-human animal:
(a) decreased exploratory behavior;
(b) decreased anxiety-related behavior;
(c) impaired long-term memory retention; and
(d) increased escape latency.

44. The non-human animal of any one of claims 34-43, wherein the non-human animal displays all of the following relative to a wild type non-human animal:
(a) decreased exploratory behavior;
(b) decreased anxiety-related behavior;
(c) impaired long-term memory retention; and
(d) increased escape latency.

45. The non-human animal of claim 34, wherein a region comprising exon 2 to the stop codon of the endogenous Idua gene is inverted and flanked by recombinase recognition sites in the reverse-conditional null endogenous Idua gene,
wherein the recombinase recognition sites are oriented such that the region is reinverted upon treatment with the recombinase,
wherein the recombinase is a Cre recombinase, and the recombinase recognition sites comprise a lox71 site and a lox66 site,
wherein the non-human animal comprises the reverse-conditional null endogenous Idua gene in its germline,
wherein the non-human animal is a mouse, and
wherein the non-human animal displays one or more phenotypes associated with mucopolysaccharidosis I.

46. The non-human animal of claim 45, wherein the non-human animal displays one or more skeletal phenotypes associated with mucopolysaccharidosis I.

47. The non-human animal of claim 45, wherein the non-human animal displays one or more neurological phenotypes associated with mucopolysaccharidosis I.

48. The non-human animal of claim 45, wherein the non-human animal displays one or more skeletal phenotypes associated with mucopolysaccharidosis I and one or more neurological phenotypes associated with mucopolysaccharidosis I.

49. The non-human animal of any one of claims 45-48, wherein the non-human animal displays one or more or all of the following relative to a wild type non-human animal:
(a) increased accumulation of glycosaminoglycans in the liver;
(b) increased accumulation of glycosaminoglycans in the heart;
(c) increased accumulation of glycosaminoglycans in the kidney;
(d) decreased tibia length;
(e) decreased spinal column length;
(f) decreased ribcage width; and
(g) decreased cranial length:width ratio.

50. The non-human animal of any one of claims 45-49, wherein the non-human animal displays one or more or all of the following relative to a wild type non-human animal:
(a) decreased exploratory behavior;
(b) decreased anxiety-related behavior;
(c) impaired long-term memory retention; and
(d) increased escape latency.

51. The non-human animal of any one of claims 34-50, wherein the reverse-conditional null endogenous Idua gene comprises the sequence set forth in SEQ ID NO: 10, 11, or 49.

52. The non-human animal of any one of claims 34-51, wherein upon treatment the reverse-conditional null endogenous Idua gene comprises the sequence set forth in SEQ ID NO: 12 or 50.

53. A non-human animal cell comprising in its genome a reverse-conditional null endogenous lysosomal storage disease gene, wherein the reverse-conditional null endogenous lysosomal storage disease gene is a null endogenous lysosomal storage disease gene that is converted to a functional endogenous lysosomal storage disease gene upon treatment with a recombinase.

54. A non-human animal genome comprising a reverse-conditional null endogenous lysosomal storage disease gene, wherein the reverse-conditional null endogenous lysosomal storage disease gene is a null endogenous lysosomal storage disease gene that is converted to a functional endogenous lysosomal storage disease gene upon treatment with a recombinase.

55. A nucleic acid comprising a reverse-conditional null endogenous lysosomal storage disease gene, wherein the reverse-conditional null endogenous lysosomal storage disease gene is a null endogenous lysosomal storage disease gene that is converted to a functional endogenous lysosomal storage disease gene upon treatment with a recombinase.

56. A reverse-conditional null endogenous lysosomal storage disease gene,
wherein the reverse-conditional null endogenous lysosomal storage disease gene is a null endogenous lysosomal storage disease gene that is converted to a functional endogenous lysosomal storage disease gene upon treatment with a recombinase.

57. A method of assessing reversibility of a phenotype of a lysosomal storage disease, comprising:
(a) treating a non-human animal of any one of claims 1-52 with the recombinase at a selected time point after birth or treating the non-human animal with a candidate therapeutic agent at the selected time point after birth; and
(b) assessing the phenotype of the lysosomal storage disease after a defined time period post-treatment relative to a control non-human animal.

58. The method of claim 57, wherein step (a) comprises treating the non-human animal with the candidate therapeutic agent at the selected time point after birth.

59. The method of claim 58, wherein the candidate therapeutic agent is a protein encoded by the endogenous lysosomal storage disease gene or a nucleic acid encoding the protein.

60. The method of claim 58 or 59, wherein the control non-human animal is the non-human animal in step (a) prior to treatment with the candidate therapeutic agent.

61. The method of any one of claims 58-60, wherein the control non-human animal is a second non-human animal comprising the reverse-conditional null endogenous lysosomal storage disease gene, wherein the second non-human animal has not been treated with the candidate therapeutic agent.

62. The method of claim 61, wherein the second non-human animal is the same age as the non-human animal in step (a).

63. The method of claim 57, wherein step (a) comprises treating the non-human animal with the recombinase at the selected time point after birth.

64. The method of claim 63, wherein the non-human animal further comprises a genomically integrated recombinase expression cassette encoding the recombinase, wherein the recombinase is inducible, and step (a) comprises inducing the recombinase.

65. The method of claim 64, wherein the recombinase is inducible upon treatment with tamoxifen, and step (a) comprises treating the non-human animal with tamoxifen.

66. The method of any one of claims 63-65, wherein the control non-human animal is the non-human animal in step (a) prior to treatment with the recombinase.

67. The method of any one of claims 63-65, wherein the control non-human animal is a second non-human animal comprising the reverse-conditional null endogenous lysosomal storage disease gene, wherein the second non-human animal has not been treated with the recombinase.

68. The method of claim 67, wherein the second non-human animal is the same age as the non-human animal in step (a).

69. The method of any one of claims 57-68, wherein step (b) further comprises assessing the phenotype of the lysosomal storage disease after a defined time period post-treatment relative to a wild type non-human animal.

70. The method of claim 69, wherein the wild type non-human animal is the same age as the non-human animal in step (b).

71. The method of any one of claims 57-70, wherein the defined time period post-treatment in step (b) is at least 1 week, at least 1 month, or at least 3 months after treatment.

72. The method any one of claims 57-71, wherein the defined time period post-treatment in step (b) is between about 1 month and about 3 months after treatment.

73. The method of any one of claims 57-72, wherein the lysosomal storage disease gene is a mucopolysaccharidosis gene, and the phenotype is a mucopolysaccharidosis phenotype.

74. The method of any one of claims 57-73, wherein the lysosomal storage disease gene is Arsb, and the phenotype is a mucopolysaccharidosis VI phenotype.

75. The method of claim 74, wherein the phenotype is a skeletal mucopolysaccharidosis VI phenotype.

76. The method of claim 74 or 75, wherein the phenotype comprises, relative to a wild type non-human animal:
(i) increased accumulation of glycosaminoglycans in the liver;
(ii) increased accumulation of glycosaminoglycans in the heart;
(iii) increased accumulation of glycosaminoglycans in the kidney;
(iv) decreased tibia length;
(v) decreased spinal column length;
(vi) increased tibial growth plate width; or
(vii) decreased cranial length:width ratio.

77. The method of any one of claims 74-76, wherein the phenotype comprises the decreased tibia length, the decreased spinal column length, the increased tibial growth plate width, or the decreased cranial length:width ratio.

78. The method of any one of claims 57-73, wherein the lysosomal storage disease gene is Idua, and the phenotype is a mucopolysaccharidosis I phenotype.

79. The method of claim 78, wherein the phenotype is a skeletal mucopolysaccharidosis I phenotype.

80. The method of claim 78 or 79, wherein the phenotype comprises, relative to a wild type non-human animal:
(i) increased accumulation of glycosaminoglycans in the liver;
(ii) increased accumulation of glycosaminoglycans in the heart;
(iii) increased accumulation of glycosaminoglycans in the kidney;
(iv) decreased tibia length;
(v) decreased spinal column length;
(vi) decreased ribcage width; or
(vii) decreased cranial length:width ratio.

81. The method of claim 78, wherein the phenotype is a neurological mucopolysaccharidosis I phenotype.

82. The method of claim 78 or 81, wherein the phenotype comprises, relative to a wild type non-human animal:
(a) decreased exploratory behavior;
(b) decreased anxiety-related behavior;
(c) impaired long-term memory retention; or
(d) increased escape latency.

83. A method of determining an optimal timeframe for treatment of a phenotype of a lysosomal storage disease, comprising:
(a) performing the method of any one of claims 57-82 a first time in a first non-human animal, wherein the first non-human animal is treated with the recombinase at a first time point after birth;
(b) performing the method of step (a) a second time in a second non-human animal, wherein the second non-human animal is treated with the recombinase at a second time point after birth, wherein the second time point is different from the first time point; and
(c) comparing the phenotype in step (a) with the phenotype in step (b) and selecting the time point resulting in the better amelioration of the phenotype.

84. The method of claim 83, wherein the lysosomal storage disease gene is a mucopolysaccharidosis gene, and the phenotype type is a mucopolysaccharidosis phenotype.

85. The method of claim 83 or 84, wherein the lysosomal storage disease gene is Arsb, and the phenotype is a mucopolysaccharidosis VI phenotype.

86. The method of claim 85, wherein the phenotype is a skeletal mucopolysaccharidosis VI phenotype.

87. The method of claim 85 or 86, wherein the phenotype comprises, relative to a wild type non-human animal:
(i) increased accumulation of glycosaminoglycans in the liver;
(ii) increased accumulation of glycosaminoglycans in the heart;
(iii) increased accumulation of glycosaminoglycans in the kidney;
(iv) decreased tibia length;
(v) decreased spinal column length;
(vi) increased tibial growth plate width; or
(vii) decreased cranial length:width ratio.

88. The method of any one of claims 85-87, wherein the phenotype comprises the decreased tibia length, the decreased spinal column length, the increased tibial growth plate width, or the decreased cranial length:width ratio.

89. The method of claim 83 or 84, wherein the lysosomal storage disease gene is Idua, and the phenotype is a mucopolysaccharidosis I phenotype.

90. The method of claim 89, wherein the phenotype is a skeletal mucopolysaccharidosis I phenotype.

91. The method of claim 89 or 90, wherein the phenotype comprises, relative to a wild type non-human animal:
(i) increased accumulation of glycosaminoglycans in the liver;
(ii) increased accumulation of glycosaminoglycans in the heart;
(iii) increased accumulation of glycosaminoglycans in the kidney;
(iv) decreased tibia length;
(v) decreased spinal column length;
(vi) decreased ribcage width; or
(vii) decreased cranial length:width ratio.

92. The method of claim 89, wherein the phenotype is a neurological mucopolysaccharidosis I phenotype.

93. The method of claim 89 or 92, wherein the phenotype comprises, relative to a wild type non-human animal:
(a) decreased exploratory behavior;
(b) decreased anxiety-related behavior;
(c) impaired long-term memory retention; or
(d) increased escape latency.

94. A method of assessing the reversibility of a phenotype of a lysosomal storage disease by a combination therapy, comprising:
(a) performing the method of any one of claims 57-82 a first time in a first non-human animal and a second time in a second non-human animal,
wherein the first non-human animal is treated with the recombinase but not the candidate therapeutic agent, and
wherein the second non-human animal is treated with the recombinase and the candidate therapeutic agent; and
(b) comparing the phenotype in the first non-human animal with the phenotype in the second non-human animal to determine if the combination of the recombinase and the candidate therapeutic agent results in better amelioration of the phenotype.

95. The method of claim 94, wherein the second non-human animal is treated with the recombinase and the candidate therapeutic agent simultaneously.

96. The method of claim 94, wherein the second non-human animal is treated with the recombinase and the candidate therapeutic agent sequentially.

97. The method of any one of claims 94-96, wherein the candidate therapeutic agent is not a protein encoded by the endogenous lysosomal storage disease gene or a nucleic acid encoding the protein.

98. A method of assessing the efficacy of a candidate therapeutic agent for reversing a phenotype of a lysosomal storage disease, comprising:
(a) performing the method of any one of claims 57-82 a first time in a first non-human animal and a second time in a second non-human animal,
wherein the first non-human animal is treated with the recombinase but not the candidate therapeutic agent, and
wherein the second non-human animal is treated with the candidate therapeutic agent but not the recombinase; and
(b) comparing the phenotype in the first non-human animal with the phenotype in the second non-human animal to determine if the recombinase results in better amelioration of the phenotype than the candidate therapeutic agent.

99. The method of claim 98, wherein the recombinase and the candidate therapeutic agent are administered at the same selected time point after birth, and the phenotype is assessed after the same defined time period post-treatment in the first non-human animal and the second non-human animal.

100. The method of claim 98 or 99, wherein the candidate therapeutic agent is a protein encoded by the endogenous lysosomal storage disease gene or a nucleic acid encoding the protein.

101. A method of making the non-human animal of any one of claims 1-52, comprising:
(a) introducing into a non-human animal host embryo a genetically modified non-human animal embryonic stem (ES) cell comprising in its genome the reverse-conditional null endogenous lysosomal storage disease gene; and
(b) gestating the non-human animal host embryo in a surrogate non-human animal mother, wherein the surrogate non-human animal mother produces an F0 progeny genetically modified non-human animal comprising the reverse-conditional null endogenous lysosomal storage disease gene.

102. The method of claim 101, further comprising modifying the non-human animal ES cell prior to step (a) to comprise in its genome the reverse-conditional null endogenous lysosomal storage disease gene.

103. A method of making the non-human animal of any one of claims 1-52, comprising gestating a genetically modified non-human animal host embryo in a non-human animal surrogate mother, wherein the genetically modified non-human animal host embryo comprises in its genome the reverse-conditional null endogenous lysosomal storage disease gene, and wherein the surrogate non-human animal mother produces an F0 progeny genetically modified non-human animal comprising the reverse-conditional null endogenous lysosomal storage disease gene.

104. The method of claim 103, further comprising modifying a non-human animal one-cell stage embryo to generate the genetically modified non-human animal host embryo prior to the gestating step.
